(19) United States
(12) Patent Application Publication
(10) Pub. No.: US 2026/0250276 A1
(43) Pub. Date: Aug. 27, 2026

(54) PROLINE DERIVATIVES AND USES THEREOF

(71) Applicant: Pfizer Inc., New York, NY (US)

(72) Inventors: Kevin James Filipski, Reading, MA (US); Matthew Richard Reese, Mystic, CT (US)

(73) Assignee: Pfizer Inc., New York, NY (US)

(21) Appl. No.: 19/546,635

(22) Filed: Feb. 23, 2026

Related U.S. Application Data
(60) Provisional application No. 63/762,915, filed on Feb. 25, 2025.

(51) Int. Cl. C07D 417/12; A61K 31/427; A61K 31/4439; C07D 401/12; C07D 401/14

(52) U.S. Cl. CPC C07D 417/12; A61K 31/427; A61K 31/4439; C07D 401/12; C07D 401/14

(57) ABSTRACT

The present disclosure relates to novel proline derivatives including pharmaceutically acceptable salts thereof; to their use in medicine; to pharmaceutical compositions containing them; to processes for their preparation; and to intermediates used in such processes. The novel proline derivatives may be useful in the treatment and/or prevention of, for example, obesity, weight gain, and/or T2DM.

This application claims the benefit of priority to U.S. Provisional Patent Application Ser. No. 63/762,915 filed Feb. 25, 2025, the disclosure of which is hereby incorporated by reference in its entirety.

### FIELD OF THE INVENTION

The present invention relates to new pharmaceutical compounds, pharmaceutical compositions containing the compounds, and use of the compounds as glucose-dependent insulinotropic polypeptide receptor (GIPR) antagonists.

### BACKGROUND OF THE INVENTION

Glucose-dependent insulinotropic polypeptide (GIP, formerly called gastric inhibitory polypeptide) is a 42-amino acid peptide secreted from K-cells in the small intestine (duodenum and jejunum). Human GIP is derived from the processing of proGIP, a 153-amino acid precursor encoded by a gene localized on chromosome 17 (See e.g., Inagaki et al., Mol Endocrinol 1989; 3:1014-1021; and Fehmann et al. Endocr Rev. 1995; 16:390-410). GIP secretion is induced by food ingestion. GIP is a known insulinotropic factor (or “incretin”) that enhances glucose-dependent insulin secretion. GIP has additional physiological effects in multiple tissues, including the promotion of fat storage in the adipose. Intact GIP is rapidly inactivated by dipeptidyl peptidase 4 (DPPIV).

The GIP receptor (GIPR) belongs to the glucagon subfamily of class 1 G protein-coupled receptors (GPCRs) characterized by an extracellular N-terminal domain, seven transmembrane domains and an intracellular C-terminus (See e.g. Zhao et al. Nat Commun. 2022, 13:1057). The N-terminal extracellular domain forms the primary peptide recognition and binding site of the receptor.

Upon stimulation with GIP, GIPR undergoes structural changes from inactive to active conformations, thereby triggering a Gas-mediated increase in cAMP production. GIPR is expressed in various tissues, including the pancreas, gut, adipose tissue, vasculature, heart, and brain (see e.g. Hammoud et al. Nat Rev Endocrinol 2023; 18: 201-216). Human GIPR comprises 466 amino acids and is encoded by a gene located on chromosome 19 (see e.g. Gremlich et al., Diabetes. 1995; 44:1202-8; and Volz et al., FEBS Lett. 1995, 373:23-29). Studies suggest that alternative mRNA splicing results in the production of GIPR variants with differing length (see e.g., Harada et al. Am J Physiol Endocrinol Metab. 2008. 294: E61-E68; and Marti-Solano et al. Nature. 2020, 587: 650-656).

GIPR knockout mice are resistant to high fat diet-induced weight gain and have improved insulin sensitivity and lipid profiles (see e.g. Yamada et al. Diabetes. 2006, 55:S86; and Miyawaki et al. Nature Med. 2002, 8:738-742). Recent data supports that heterozygous loss of function in GIPR results in lower BMI and obesity risk in humans (see e.g. Akbari et al. Science. 2021, 373: 6550). Small molecules, peptides, and monoclonal antibodies with antagonist activity at GIPR have been shown to prevent weight gain and insulin resistance in preclinical obesity models (see e.g. Nakamura et al. Diabetes Metab Syndr Obes. 2021,14:1095-1105; Yang et al. Mol Metab. 2022, 66: 101638; and Killion et al. Sci. Transl. Med., 2018, 10:eaat3392). The combination of GIPR modulators with GLP-1R agonists has been associated with superior weight loss (see e.g. Lu et al. Cell Rep Med. 2021, 2(5):100263). Collectively, these links to obesity and metabolic diseases suggest that GIPR inhibition is a useful approach for therapeutic intervention, both as monotherapy and in combination with other agents including GLP-1R agonists. Moreover, human epicardial adipose tissue—which plays a crucial role in the development and progression of coronary artery disease, atrial fibrillation, and heart failure—has been found to express GIPR genes and proteins. See e.g. Malavazos et al., European Journal of Preventive Cardiology (2023) 00, 1-14.

There continues to be a need for alternative GIPR antagonists, for example, for developing new and/or improved pharmaceuticals (e.g., more effective, more selective, less toxic, improved patient compliance, and/or having improved biopharmaceutical properties such as physical stability; solubility; oral bioavailability; appropriate metabolic stability; clearance; half life) to treat or prevent GIPR-related conditions, diseases, or disorders, such as those described herein. The present invention is directed to these and other important ends.

### SUMMARY OF THE INVENTION

In one embodiment (Embodiment A1), the present invention provides a compound of Formula I:

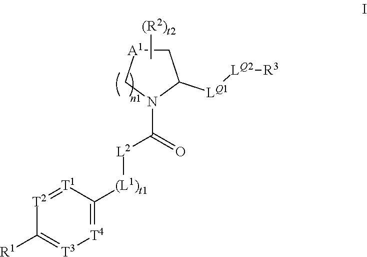

or a pharmaceutically acceptable salt thereof, wherein:
R¹ is H, halogen, —CN, —OR^{1C}, C₁₋₈alkyl, C₁₋₈ haloalkyl, C₂₋₈ alkenyl, —C₁₋₄ alkyl-(C₃₋₆ cycloalkyl), or C₃₋₆ cycloalkyl, wherein each of the C₁₋₈ alkyl, C₂₋₈ alkenyl, —C₁₋₄ alkyl-(C₃₋₆ cycloalkyl), or C₃₋₆ cycloalkyl is optionally substituted with 1, 2, 3, 4, 5, or 6 substituents each independently selected from halogen, —OH, —CN, C₁₋₄ alkyl, C₁₋₄ haloalkyl, C₁₋₄ alkoxy, and C₁₋₄ haloalkoxy;
R^{1C} is C₁₋₆ alkyl, C₁₋₆ haloalkyl, C₃₋₆ cycloalkyl, or —C₁₋₂ alkyl-(C₃₋₆ cycloalkyl), wherein each of the C₃₋₆ cycloalkyl and —C₁₋₂ alkyl-(C₃₋₆ cycloalkyl) is optionally substituted with 1, 2, 3, 4, 5, or 6 substituents each independently selected from halogen, —OH, —CN, C₁₋₄ alkyl, C₁₋₄ haloalkyl, C₁₋₄ alkoxy, and C₁₋₄ haloalkoxy;
each of T¹, T², T³, and T⁴ is independently CR⁴ or N, provided that only 0, 1, or 2 of T¹, T², T³, and T⁴ is N;
each R⁴ is independently H, halogen, —CN, C₃₋₆ cycloalkyl, —C₁₋₂ alkyl-(C₃₋₆ cycloalkyl), C₁₋₄ alkyl, C₁₋₄ cyanoalkyl, C₁₋₄ haloalkyl, C₁₋₄ alkoxy, or C₁₋₄ haloalkoxy;
or R¹ and an adjacent R⁴, together with the two ring carbon atoms to which they are attached, optionally form a fused 4- or 6-membered cycloalkyl ring, a fused 4- or 6-membered heterocycloalkyl ring, a fused 5- or 6-membered heteroaryl ring, or a fused 6-membered aryl ring, wherein each of the fused rings is optionally substituted with 1, 2, 3, 4, 5, or 6 substituents each independently selected from halogen, —OH, —CN, C₁₋₄ alkyl, C₁₋₄ haloalkyl, C₁₋₄ alkoxy, and C₁₋₄ haloalkoxy;
t1, L¹, and L² are
   (a) t1 is 0 or 1, L¹ is C(R^{L1})₂ or [C(R^{L1})₂]₂, and L² is NR^{N}; or
   (b) t1 is 0 or 1, L¹ is C(R^{L1})₂, O, or NR^{N}, and L² is C(R^{L2})₂; or
   (c) t1 is 1, and -L¹-L²-is —C(R^{L1})₂—O—C(R^{L2})₂—, [C(R^{L1})₂]₃, or —C(R^{L1})₂—N(R^{N})—C(R^{L2})₂—, or -L¹-L²- together form a divalent C₃₋₆ cycloalkyl ring optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy;
each of R^{L1} and R^{L2} is independently H, C₁₋₂ alkyl, C₁₋₂ haloalkyl, C₁₋₂ alkoxy, or C₁₋₂ haloalkoxy;
or two R^{L1}, together with the carbon atom to which they are attached, optionally form C₃₋₆ cycloalkyl or a 3- to 6-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
or two R^{L2}, together with the carbon atom to which they are attached, optionally form C₃₋₆ cycloalkyl or a 3- to 6-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
or —C(R^{L1})₂—C(R^{L2})₂— together optionally form C₃₋₆ cycloalkyl or a 4- to 6-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
R^{N} is H, C₁₋₆ alkyl, C₃₋₆ cycloalkyl, —C₁₋₄ alkyl-(C₃₋₆ cycloalkyl);
n1 and A¹ are
   (i) n1 is 1 and A¹ is CH₂; or
   (ii) n1 is 2 and A¹ is CH₂, O, S, or NH;
each R² is independently halogen, —OH, C₁₋₄ alkyl, C₁₋₄ haloalkyl, C₁₋₄ alkoxy, C₁₋₄ haloalkoxy, C₃₋₄ cycloalkyl, or —C₁₋₄ alkyl-(C₃₋₆ cycloalkyl), wherein each of the C₁₋₄ alkyl, C₁₋₄ haloalkyl, C₁₋₄ alkoxy, C₁₋₄ haloalkoxy, C₃₋₄ cycloalkyl, or —C₁₋₄ alkyl-(C₃₋₆ cycloalkyl) is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy, provided that when a R² is attached to a ring-forming nitrogen atom of the ring having the variable n1, then the R² is not halogen, —OH, an optionally substituted C₁₋₄ alkoxy, or an optionally substituted C₁₋₄ haloalkoxy;
or two R², when attached to a same ring-forming carbon atom of the ring having the variable n1, together with the ring carbon atom to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
or two R², when attached to two adjacent ring-forming atoms of the ring having the variable n1, together with the two ring-forming atoms to which they are attached, optionally form a fused C₃₋₆ cycloalkyl or a fused 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
t2 is 0, 1, 2, 3, or 4;
L^{Q1} and L^{Q2} are:
   (x) L^{Q1} is C(R^{Q1})₂, and L^{Q2} is O; or
   (y) L^{Q1} is C(═O), and L^{Q2} is NH;
each R^{Q1} is independently H, C₁₋₂ alkyl, C₁₋₂ haloalkyl, C₃₋₄ cycloalkyl, or —C₁₋₂ alkyl-(C₃₋₄ cycloalkyl);
or two R^{Q1}, together with the carbon atom to which they are attached, optionally form C₃₋₆ cycloalkyl, which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
R³ is R^{3a}, R^{3b}, R^{3c}, R^{3d}, or R^{3e}:

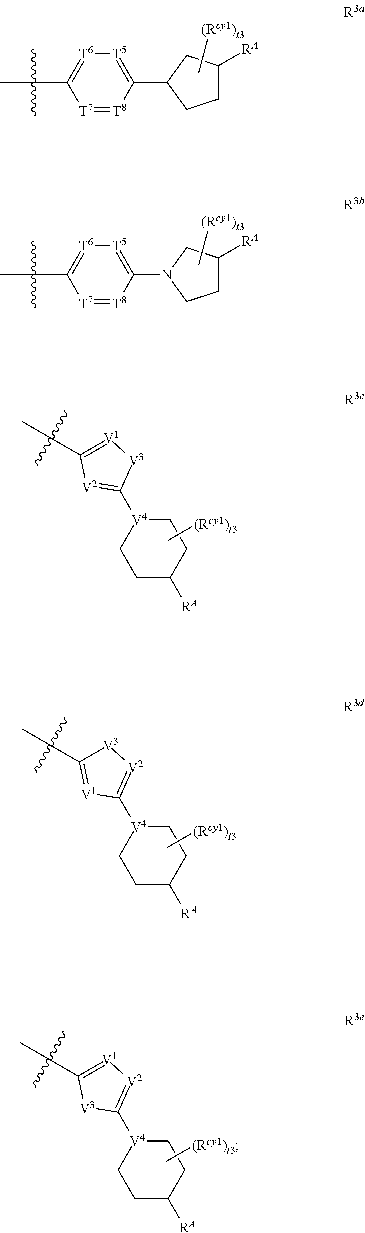

each R^{cy1} is independently halogen, —OH, C₁₋₄ alkyl, C₁₋₄ haloalkyl, C₁₋₄ hydroxylalkyl, C₁₋₄ alkoxy, C₁₋₄ haloalkoxy, C₃₋₄ cycloalkyl, or —C₁₋₄ alkyl-(C₃₋₄ cycloalkyl);
or two R^{cy1}, together with a same ring carbon atom of the cyclohexyl ring to which they are attached, form C₃₋₆ cycloalkyl that is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or —C₁₋₄ alkyl-(C₃₋₄ cycloalkyl);
or two R^{cy1}, each of which is attached to a different ring carbon atom, are linked together form a single bond or a moiety of —CH₂—, —CH₂CH₂—, —CH₂CH₂CH₂—, or —CH₂CH₂CH₂—, which moiety is optionally substituted with 1, 2, 3, or 4 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or —C₁₋₄ alkyl-(C₃₋₄ cycloalkyl);
each of V¹ and V² is independently N or CR^{S1};
V³ is O or S;
V⁴ is CH or N;
each R^{S1} is independently H, C₁₋₄ alkyl, C₁₋₄ haloalkyl, C₃₋₄ cycloalkyl, —C₁₋₄ alkyl-(C₃₋₄ cycloalkyl), C₁₋₄ alkoxy, or C₁₋₄ haloalkoxy;
t3 is 0, 1, 2, or 3;
each of T⁵, T⁶, T⁷, and T³ is independently CR⁵ or N, provided that only 0, 1, or 2 of T⁵, T⁶, T⁷, and T³ can be N;
each R⁵ is independently H, halogen, —CN, C₃₋₆ cycloalkyl, (C₃₋₆ cycloalkyl)—C₁₋₂ alkyl-, C₁₋₄ alkyl, C₁₋₄ cyanoalkyl, C₁₋₄ haloalkyl, C₁₋₄ alkoxy, or C₁₋₄ haloalkoxy; RA is —C(═O)—OH, —C(R^{L3})₂—C(═O)—OH, —C(R^{L3})₂—C(R^{L4})₂—C(═O)—OH, —[C(R^{L3})₂]₃—C(═O)—OH, —O—C(R^{L3})₂—C(═O)—OH, —O—C(R^{L3})₂—C(R^{L4})₂—C(═O)—OH, —O—[C(R^{L3})₂]₃—C(═O)—OH, OH, —C(═O)NH—C(R^{L3})₂—C(═O)—OH, —C(═O)NH—C(R^{L3})₂—C(R^{L4})₂—C(═O)—OH, —C(═O)NH—[C(R^{L3})₂]₃—C(═O)—OH, —C(═O)—N(R^{am1})(R^{am2}), —C(═O)—OR^{es1}, 1H-tetrazol-5-yl, 3-hydroxyisoxazol-5-yl, 5(4H)-oxo-1,2,4-oxadiazol-3-yl-, 5(4H)-oxo-1,2,4-thiadiazol-3-yl-, 2-thioxo-1,3,4-oxadiazol-5-yl-, 4H-1,2,4-triazol-3-yl-, 4-hydroxy-1,2,5-oxadiazol-3-yl, 1-hydroxypyrazol-5-yl, 3-hydroxy-1H-pyrazol-1-yl-, a carboxylic acid bioisostere group, —S(═O)₂NHCF₃, or —C(═O)—NH—S(═O)₂—R¹⁰⁰ wherein R¹⁰⁰ is C₁₋₆ alkyl or phenyl and where the phenyl is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
each of R^{L3} and R^{L4} is independently H, C₁₋₂ alkyl, C₁₋₂ haloalkyl, C₁₋₂ alkoxy, or C₁₋₂ haloalkoxy;
or two R^{L3}, together with the carbon atom to which they are attached, optionally form C₃₋₆ cycloalkyl or a 3- to 6-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
or two R^{L4}, together with the carbon atom to which they are attached, optionally form C₃₋₆ cycloalkyl or a 3- to 6-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
or —C(R^{L3})₂—C(R^{L4})₂— together optionally forms a divalent C₃₋₆ cycloalkyl or a divalent 4- to 6-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
each of R^{am1} and R^{am2} is independently H, C₁₋₆ alkyl, C₃₋₆ cycloalkyl, —C₁₋₄ alkyl-(C₃₋₆ cycloalkyl), phenyl, or —C₁₋₄ alkyl-phenyl, wherein each of the C₁₋₆ alkyl, C₃₋₆ cycloalkyl, C₁₋₄ alkyl-(C₃₋₆ cycloalkyl), phenyl, or —C₁₋₄ alkyl-phenyl is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or —C₁₋₄ alkyl-(C₃₋₄ cycloalkyl);
or R^{am1} and R^{am2} together with the nitrogen atom to which they are attached form a 4- to 8-membered heterocycloalkyl optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or —C₁₋₄ alkyl-(C₃₋₄ cycloalkyl), wherein each of the C₁₋₄ alkyl, C₁₋₄ haloalkyl, C₁₋₄ alkoxy, C₁₋₄ haloalkoxy, C₃₋₄ cycloalkyl, or —C₁₋₄ alkyl-(C₃₋₄ cycloalkyl) is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy; and
R^{es1} is C₁₋₆ alkyl, C₃₋₆ cycloalkyl, —C₁₋₄ alkyl-(C₃₋₆ cycloalkyl), phenyl, or —C₁₋₄ alkyl-phenyl, each of which is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or —C₁₋₄ alkyl-(C₃₋₄ cycloalkyl).

The present invention also provides a pharmaceutical composition containing the compound of Formula I or a pharmaceutically acceptable salt of the compound and a pharmaceutically acceptable excipient or carrier.

The present invention also provides a method for treating or preventing a GIPR-related condition, disease, or disorder in a patient (e.g., a mammal or a human), which method includes administering to the patient (e.g., the mammal or human) the compound of Formula I or a pharmaceutically acceptable salt of the compound; or a method for weight management of a human, which method includes administering to the human the compound of Formula I or a pharmaceutically acceptable salt of the compound.

The present invention also provides the compound of Formula I or a pharmaceutically acceptable salt of the compound for use in treating or preventing a GIPR-related condition, disease, or disorder, or for use in weight management.

The present invention also provides use of the compound of Formula I or a pharmaceutically acceptable salt of the compound in treating or preventing a GIPR-related condition, disease, or disorder, or in weight management.

The present invention also provides use of the compound of Formula I or a pharmaceutically acceptable salt of the compound in manufacturing a medicament for treating or preventing a GIPR-related condition, disease, or disorder, for weight management.

The GIPR-related condition, disease, or disorder includes one selected from diabetes [e.g.

Type 1 diabetes mellitus (T1D), Type 2 diabetes mellitus (T2DM), including pre-diabetes], idiopathic T1D (Type 1b), latent autoimmune diabetes in adults (LADA), early-onset T2DM (EOD), youth-onset atypical diabetes (YOAD), maturity onset diabetes of the young (MODY), malnutrition-related diabetes, gestational diabetes, hyperglycemia, insulin resistance, hepatic insulin resistance, impaired glucose tolerance, diabetic neuropathy, diabetic nephropathy, kidney disease [e.g., acute kidney disorder, tubular dysfunction, proinflammatory changes to the proximal tubules, or chronic kidney disease (CKD)], diabetic retinopathy, adipocyte dysfunction, visceral adipose deposition, sleep apnea [e.g. obstructive sleep apnea (OSA)], obesity (including hypothalamic obesity and monogenic obesity) and related comorbidities (e.g., osteoarthritis and urine incontinence), eating disorders (including binge eating syndrome, bulimia nervosa, and syndromic obesity such as Prader-Willi and Bardet-Biedl syndromes), weight gain such as weight gain caused by use of other agents (e.g., caused by use of steroids and/or antipsychotics, or caused by treatment of depression, or caused by use of agents on cognitive function), excessive sugar craving, dyslipidemia [including hyperlipidemia, hypertriglyceridemia, increased total cholesterol, high LDL (low-density lipoprotein) cholesterol, and low HDL (high-density lipoprotein) cholesterol], hyperinsulinemia, nonalcoholic fatty liver disease [NAFLD, including related diseases such as steatosis, nonalcoholic steatohepatitis (NASH), fibrosis, cirrhosis, and hepatocellular carcinoma], cardiovascular disease, atherosclerosis (including coronary artery disease), peripheral vascular disease, hypertension, endothelial dysfunction, impaired vascular compliance, heart failure [e.g. congestive heart failure, heart failure with preserved ejection fraction (HFpEF), heart failure with reduced ejection fraction (HFrEF)], myocardial infarction (e.g. necrosis and apoptosis), stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, osteoarthritis, Parkinson's disease, left ventricular hypertrophy, peripheral arterial disease, macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, polycystic ovary syndrome (PCOS), and addiction (e.g., addition to alcohol, nicotine, and/or drug).

The present invention also provides a method for antagonizing a glucose-dependent insulinotropic polypeptide receptor (GIPR), which method includes contacting the GIPR with the compound of Formula I or a pharmaceutically acceptable salt of the compound.

It is to be understood that both the foregoing general description and the following detailed description are exemplary and explanatory only and are not restrictive of the invention, as claimed.

### DETAILED DESCRIPTION OF THE INVENTION

The present invention may be understood more readily by reference to the following detailed description of exemplary embodiments of the invention and the examples included therein.

It is to be understood that this invention is not limited to specific synthetic methods of making that may of course vary. It is to be also understood that the terminology used herein is for the purpose of describing specific embodiments only and is not intended to be limiting.

A1 A compound of Formula I or a pharmaceutically acceptable salt thereof, as defined above.

In some further embodiments, L^{Q1} is C(R^{Q1})₂ and L^{Q2} is O.

In some other further embodiments, L^{Q1} is C(═O) and L^{Q2} is NH.

A2. The compound of embodiment A1 (including any further embodiment thereof), wherein the compound of Formula I is a Formula Ia:

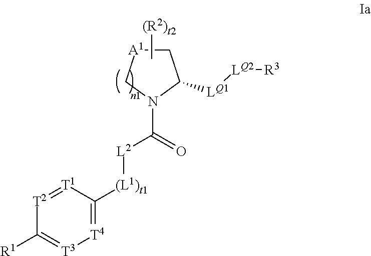

or a pharmaceutically acceptable salt thereof.
A3. The compound of embodiment A1 (including any further embodiment thereof), wherein the compound of Formula I is a Formula II:

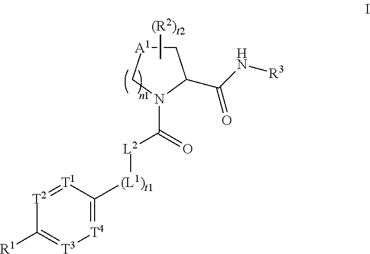

or a pharmaceutically acceptable salt thereof.
A4. The compound of embodiment A1 (including any further embodiment thereof), wherein the compound of Formula I is a Formula Ila:

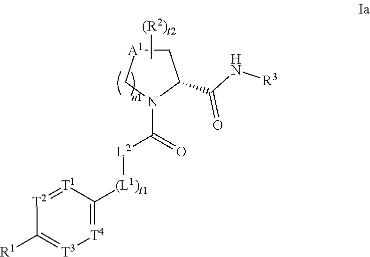

or a pharmaceutically acceptable salt thereof,
A5. The compound of embodiment A1 (including any further embodiment thereof), wherein the compound of Formula I is a compound of Formula III or Illa:

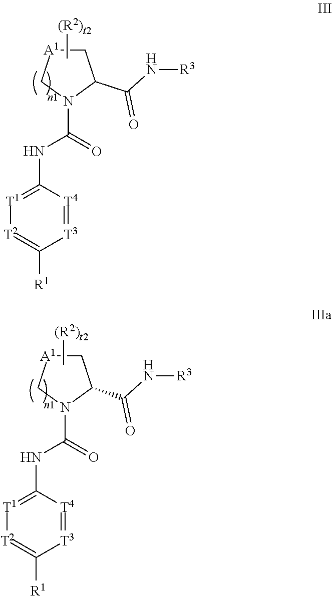

or a pharmaceutically acceptable salt thereof.

In some further embodiments, the compound of Formula I or a pharmaceutically acceptable salt thereof is a compound of Formula III or a pharmaceutically acceptable salt thereof.

In some further embodiments, the compound of Formula I or a pharmaceutically acceptable salt thereof is a compound of Formula Illa or a pharmaceutically acceptable salt thereof.

A6. The compound of embodiment A1 (including any further embodiment thereof), wherein the compound of Formula I is a compound of Formula IV or IVa:

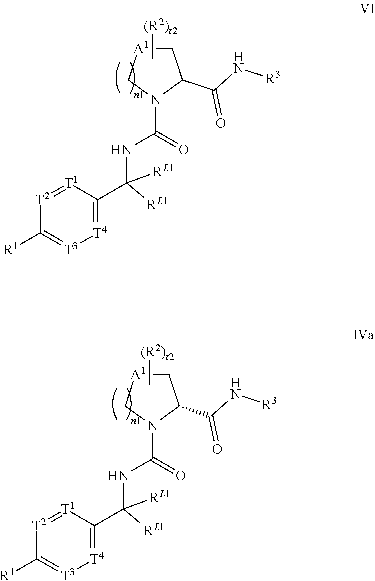

or a pharmaceutically acceptable salt thereof.

In some further embodiments, the compound of Formula I or a pharmaceutically acceptable salt thereof is a compound of Formula IV or a pharmaceutically acceptable salt thereof.

In some further embodiments, the compound of Formula I or a pharmaceutically acceptable salt thereof is a compound of Formula Va or a pharmaceutically acceptable salt thereof.

A7. The compound of embodiment A1 (including any further embodiment thereof), wherein the compound of Formula I is a compound of Formula V or Va:

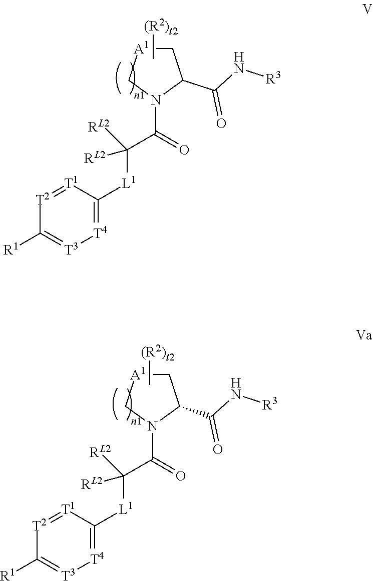

or a pharmaceutically acceptable salt thereof.

In some further embodiments, the compound of Formula I or a pharmaceutically acceptable salt thereof is a compound of Formula V or a pharmaceutically acceptable salt thereof.

In some further embodiments, the compound of Formula I or a pharmaceutically acceptable salt thereof is a compound of Formula Va or a pharmaceutically acceptable salt thereof.

A8. The compound of embodiment A1 (including any further embodiment thereof), wherein the compound of Formula I is a compound of Formula VI or VIa:

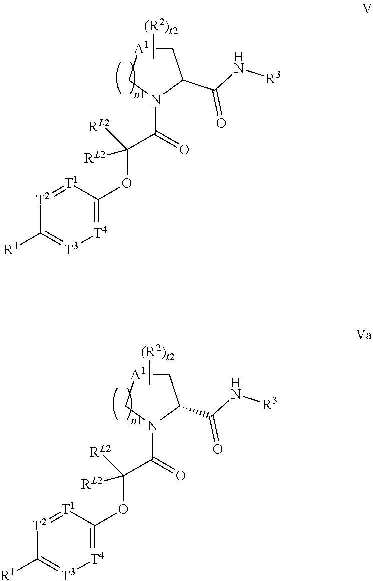

or a pharmaceutically acceptable salt thereof.

In some further embodiments, the compound of Formula I or a pharmaceutically acceptable salt thereof is a compound of Formula VI or a pharmaceutically acceptable salt thereof.

In some further embodiments, the compound of Formula I or a pharmaceutically acceptable salt thereof is a compound of Formula VIa or a pharmaceutically acceptable salt thereof.

A9. The compound of embodiment A1 (including any further embodiment thereof), wherein the compound of Formula I is a compound of Formula VII or VIIa:

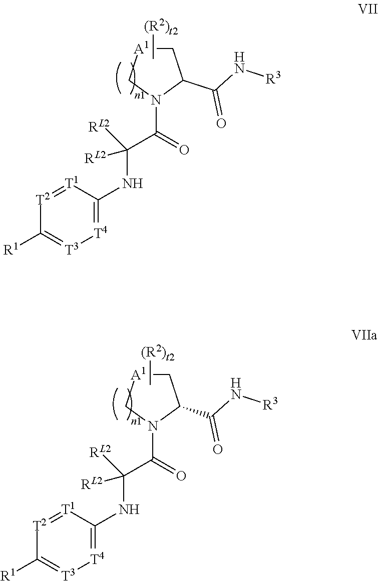

or a pharmaceutically acceptable salt thereof.

In some further embodiments, the compound of Formula I or a pharmaceutically acceptable salt thereof is a compound of Formula VII or a pharmaceutically acceptable salt thereof.

In some further embodiments, the compound of Formula I or a pharmaceutically acceptable salt thereof is a compound of Formula Vila or a pharmaceutically acceptable salt thereof.

A10. The compound of embodiment A1 (including any further embodiment thereof), wherein the compound of Formula I is a compound of Formula VIII or VIIIa:

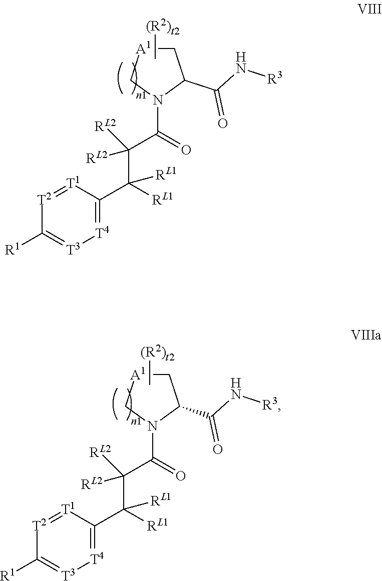

or a pharmaceutically acceptable salt thereof.

In some further embodiments, the compound of Formula I or a pharmaceutically acceptable salt thereof is a compound of Formula VIII or a pharmaceutically acceptable salt thereof.

In some further embodiments, the compound of Formula I or a pharmaceutically acceptable salt thereof is a compound of Formula Villa or a pharmaceutically acceptable salt thereof.

A11. The compound of embodiment A10 or a pharmaceutically acceptable salt thereof (including any further embodiment thereof), wherein each of R^{L1} and R^{L2} is independently H, C₁₋₂ alkyl, C₁₋₂ haloalkyl, C₁₋₂ alkoxy, or C₁₋₂ haloalkoxy.
A12. The compound of embodiment A10 or a pharmaceutically acceptable salt thereof (including any further embodiment thereof), wherein —C(R^{L1})₂—C(R^{L2})₂— together form C₃₋₆ cycloalkyl that is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy.
A13. The compound of embodiment A10 or a pharmaceutically acceptable salt thereof (including any further embodiment thereof), wherein —C(R^{L1})₂—C(R^{L2})₂— together form cyclopropyl that is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
A14. The compound of embodiment A1 (including any further embodiment thereof), wherein the compound of Formula I is a compound of Formula IX or IXa:

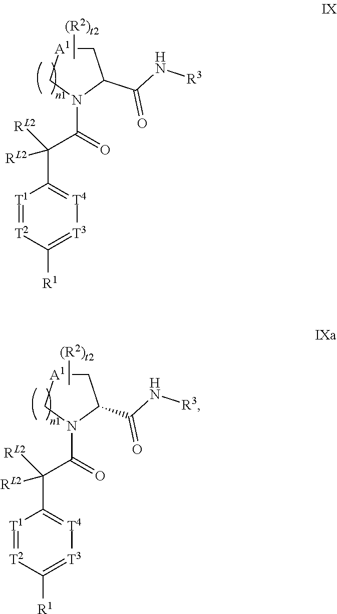

or a pharmaceutically acceptable salt thereof.

In some further embodiments, the compound of Formula I or a pharmaceutically acceptable salt thereof is a compound of Formula IX or a pharmaceutically acceptable salt thereof.

In some further embodiments, the compound of Formula I or a pharmaceutically acceptable salt thereof is a compound of Formula IXa or a pharmaceutically acceptable salt thereof.

A15. The compound of embodiment A1 (including any further embodiment thereof), wherein the compound of Formula I is a compound of Formula X or Xa:

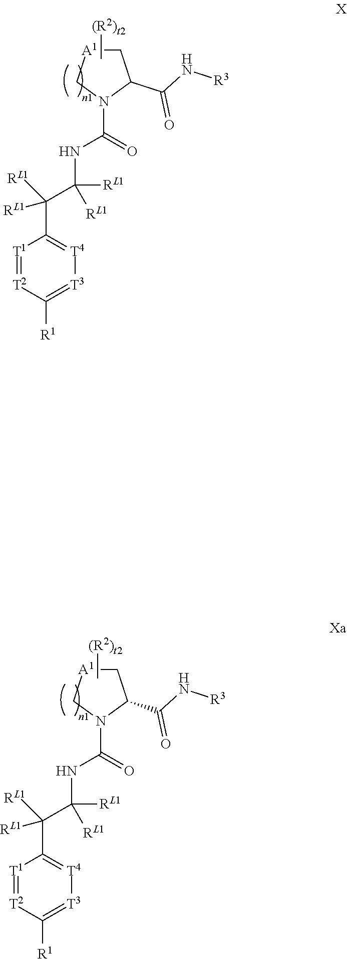

or a pharmaceutically acceptable salt thereof.

In some further embodiments, the compound of Formula I or a pharmaceutically acceptable salt thereof is a compound of Formula X or a pharmaceutically acceptable salt thereof.

In some further embodiments, the compound of Formula I or a pharmaceutically acceptable salt thereof is a compound of Formula Xa or a pharmaceutically acceptable salt thereof.

A16. The compound of embodiment A1 (including any further embodiment thereof), wherein the compound of Formula I is a compound of Formula XI or XIa:

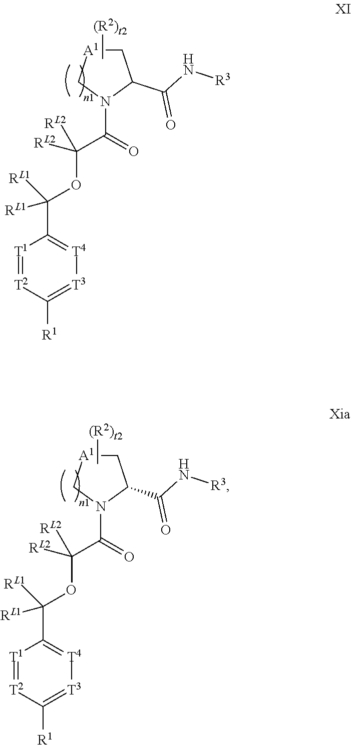

or a pharmaceutically acceptable salt thereof.

In some further embodiments, the compound of Formula I or a pharmaceutically acceptable salt thereof is a compound of Formula XI or a pharmaceutically acceptable salt thereof.

In some further embodiments, the compound of Formula I or a pharmaceutically acceptable salt thereof is a compound of Formula XIa or a pharmaceutically acceptable salt thereof.

A17. The compound of any one of embodiments A1 to A16 or a pharmaceutically acceptable salt thereof (including any further embodiment thereof), wherein R¹ is cyclopropyl, cyclobutyl, cyclopentyl, R^{1a}, R^{1b}, or R^{1c},

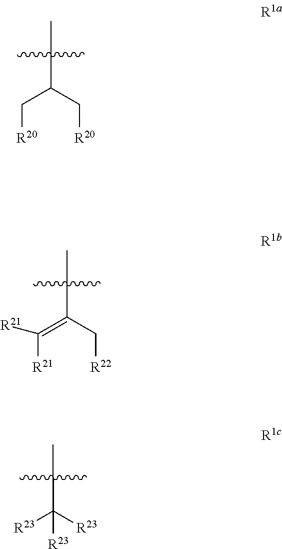

wherein each of the cyclopropyl or cyclobutyl is optionally substituted with 1, 2, 3, or 4 R^{S};
each R²⁰ is independently H, halogen, —OH, C₁₋₂ alkyl, C₁₋₂ haloalkyl, C₁₋₂ alkoxy, or C₁₋₂ haloalkoxy;
each R²¹ is independently H, C₁₋₂ alkyl, or C₁₋₂ haloalkyl;
R²² is H, halogen, C₁₋₂ alkyl, C₁₋₂ hydroxylalkyl, C₁₋₂ haloalkyl, C₁₋₂ alkoxy, or C₁₋₂ haloalkoxy;
each R²³ is independently halogen, C₁₋₂ alkyl, C₁₋₂ hydroxylalkyl, C₁₋₂ haloalkyl, C₁₋₂ alkoxy, or C₁₋₂ haloalkoxy; and
each R^{S} is independently halogen, —OH, C₁₋₂ alkyl, C₁₋₂ hydroxylalkyl, C₁₋₂ haloalkyl, C₁₋₂ alkoxy, or C₁₋₂ haloalkoxy.
   A18. The compound of any one of embodiments A1 to A16 or a pharmaceutically acceptable salt thereof (including any further embodiment thereof), wherein R¹ is propan-2-yl, prop-1-en-2-yl, or cyclopropyl.
   A19. The compound of any one of embodiments A1 to A16 or a pharmaceutically acceptable salt thereof (including any further embodiment thereof), wherein R¹ is propan-2-yl.
   A20. The compound of any one of embodiments A1 to A16 or a pharmaceutically acceptable salt thereof (including any further embodiment thereof), wherein R¹ is C₁₋₄ haloalkyl.

In some further embodiments, R¹ is C₁₋₄ fluoroalkyl.

In some further embodiments, R¹ is C₁₋₂ fluoroalkyl.

In some further embodiments, R¹ is C₁ fluoroalkyl.

A21. The compound of any one of embodiments A1 to A16 or a pharmaceutically acceptable salt thereof (including any further embodiment thereof), wherein R¹ is C₁₋₄ haloalkoxy.

In some further embodiments, R¹ is C₁₋₄ fluoroalkoxy.

In some further embodiments, R¹ is C₁₋₂ fluoroalkoxy.

In some further embodiments, R¹ is C₁ fluoroalkoxy.

A22. The compound of any one of embodiments A1 to A21 or a pharmaceutically acceptable salt thereof (including any further embodiment thereof), wherein each of T¹, T², T³, and T⁴ is independently CR⁴.
A23. The compound of any one of embodiments A1 to A21 or a pharmaceutically acceptable salt thereof (including any further embodiment thereof), wherein one of T¹, T², T³, and T⁴ is N, and the other three are each independently CR⁴.
A24. The compound of any one of embodiments A1 to A23 or a pharmaceutically acceptable salt thereof (including any further embodiment thereof), wherein each R⁴ is independently H, halogen, or C₁₋₂ alkyl.

In some further embodiments, each R⁴ is independently H or halogen.

In some further embodiments, each R⁴ is independently H or C₁₋₂ alkyl.

In some further embodiments, each R⁴ is H.

A25. The compound of any one of embodiments A1 to A24 or a pharmaceutically acceptable salt thereof (including any further embodiment thereof), wherein n1 is 1 and A¹ is CH₂.
A26. The compound of any one of embodiments A1 to A24 or a pharmaceutically acceptable salt thereof (including any further embodiment thereof), wherein n1 is 2 and A¹ is CH₂.
A27. The compound of any one of embodiments A1 to A24 or a pharmaceutically acceptable salt thereof (including any further embodiment thereof), wherein n1 is 2 and A¹ is O.
A28. The compound of any one of embodiments A1 to A27 or a pharmaceutically acceptable salt thereof (including any further embodiment thereof), wherein each R² is independently halogen, —OH, C₁₋₄ alkyl, C₁₋₄ hydroxylalkyl, C₁₋₄ haloalkyl, C₁₋₄ alkoxy, C₁₋₄ haloalkoxy, C₃₋₄ cycloalkyl, or —C₁₋₄ alkyl-(C₃₋₄ cycloalkyl); and t2 is 0, 1, or 2.

In some further embodiments, each R² is independently —OH, C₁₋₄ alkyl, C₁₋₄ hydroxylalkyl, C₁₋₄ haloalkyl, C₁₋₄ alkoxy, or C₁₋₄ haloalkoxy.

In some further embodiments, each R² is independently —OH, C₁₋₄ alkyl, or C₁₋₄ hydroxylalkyl.

In some further embodiments, each R² is independently C₁₋₄ alkyl.

A29. The compound of any one of embodiments A1 to A27 or a pharmaceutically acceptable salt thereof (including any further embodiment thereof), wherein t2 is 0.
A30. The compound of any one of embodiments A1 to A29 or a pharmaceutically acceptable salt thereof (including any further embodiment thereof), wherein R³ is R^{3a} or R^{3b}.
A31. The compound of any one of embodiments A1 to A30 or a pharmaceutically acceptable salt thereof (including any further embodiment thereof), wherein each of T⁵, T⁶, T⁷, and T³ is independently CR⁵.
A32. The compound of any one of embodiments A1 to A30 or a pharmaceutically acceptable salt thereof (including any further embodiment thereof), wherein one of T⁵, T⁶, T⁷, and T³ is N and the other three are each independently CR⁵.
A33. The compound of any one of embodiments A1 to A30 or a pharmaceutically acceptable salt thereof (including any further embodiment thereof), wherein each of T⁵, T⁶, and T⁷ is independently CR⁵ and T³ is N.
A34. The compound of any one of embodiments A1 to A33 or a pharmaceutically acceptable salt thereof (including any further embodiment thereof), wherein each R⁵ is independently H, halogen, —CN, C₁₋₄ alkyl, C₁₋₄ haloalkyl, C₁₋₄ alkoxy, or C₁₋₄ haloalkoxy.
A35. The compound of any one of embodiments A1 to A30 or a pharmaceutically acceptable salt thereof (including any further embodiment thereof), wherein T⁵ is CR⁵ wherein R⁵ is F; each of T⁶ and T⁷ is independently CR⁵ wherein each R⁵ is independently H, C₁₋₄ alkyl, or C₁₋₄ haloalkyl; and T³ is N.
A36. The compound of any one of embodiments A1 to A35 or a pharmaceutically acceptable salt thereof (including any further embodiment thereof), wherein R³ is R^{3a}.
A37. The compound of any one of embodiments A1 to A35 or a pharmaceutically acceptable salt thereof (including any further embodiment thereof), wherein R³ is R^{3b}.
A38. The compound of any one of embodiments A1 to A37 or a pharmaceutically acceptable salt thereof (including any further embodiment thereof), wherein each R^{cy1} is independently halogen, —OH, C₁₋₄ alkyl, C₁₋₄ haloalkyl, C₁₋₄ hydroxylalkyl, C₁₋₄ alkoxy, C₁₋₄ haloalkoxy, C₃₋₄ cycloalkyl, or —C₁₋₄ alkyl-(C₃₋₄ cycloalkyl); and t3 is 0 or 1.

In some further embodiments, each R^{cy1} is independently C₁₋₄ alkyl, C₁₋₄ hydroxylalkyl, C₁₋₄ alkoxy, C₁₋₄ haloalkoxy, or C₃₋₄ cycloalkyl.

In some further embodiments, each R^{cy1} is independently C₁₋₄ alkyl, or C₁₋₄ alkoxy.

In some further embodiments, each R^{cy1} is independently C₁₋₄ alkyl.

A39. The compound of any one of embodiments A1 to A37 or a pharmaceutically acceptable salt thereof (including any further embodiment thereof), wherein t3 is 0.
A40. The compound of any one of embodiments A1 to A29 or a pharmaceutically acceptable salt thereof (including any further embodiment thereof), wherein R³ is R^{3c}.
A41. The compound of embodiment A40 or a pharmaceutically acceptable salt thereof (including any further embodiment thereof), wherein each of V¹ is CR^{S1}; V² is N; and V³ is S.
A42. The compound of embodiment A41 or a pharmaceutically acceptable salt thereof (including any further embodiment thereof), wherein R^{S1} is H or C₁₋₄ alkyl.
A43. The compound of embodiment A42 or a pharmaceutically acceptable salt thereof (including any further embodiment thereof), wherein R^{S1} is H.
A44. The compound of any one of embodiments A1 to A43 or a pharmaceutically acceptable salt thereof (including any further embodiment thereof), wherein V⁴ is CH.
A45. The compound of any one of embodiments A40 to A44 or a pharmaceutically acceptable salt thereof (including any further embodiment thereof), wherein each R^{cy1} is independently halogen, —OH, C₁₋₄ alkyl, C₁₋₄ haloalkyl, C₁₋₄ hydroxylalkyl, C₁₋₄ alkoxy, C₁₋₄ haloalkoxy, C₃₋₄ cycloalkyl, or —C₁₋₄ alkyl-(C₃₋₄ cycloalkyl); and t3 is 0 or 1.

In some further embodiments, each R^{cy1} is independently C₁₋₄ alkyl, C₁₋₄ hydroxylalkyl, C₁₋₄ alkoxy, C₁₋₄ haloalkoxy, or C₃₋₄ cycloalkyl.

In some further embodiments, each R^{cy1} is independently C₁₋₄ alkyl, or C₁₋₄ alkoxy.

In some further embodiments, each R^{cy1} is independently C₁₋₄ alkyl.

A46. The compound of any one of embodiments A40 to A44 or a pharmaceutically acceptable salt thereof (including any further embodiment thereof), wherein t3 is 0.
A47. The compound of any one of embodiments A1 to A29 or a pharmaceutically acceptable salt thereof (including any further embodiment thereof), wherein R³ is R^{3d}.
A48. The compound of any one of embodiments A1 to A29 or a pharmaceutically acceptable salt thereof (including any further embodiment thereof), wherein R³ is R^{3e}.
A49. The compound of any one of embodiments A1 to A48 or a pharmaceutically acceptable salt thereof (including any further embodiment thereof), wherein R^{A} is —C(═O)—OH, —C(R^{L3})₂—C(═O)—OH, or —C(R^{L3})₂—C(R^{L4})₂—C(═O)—OH.
A50. The compound of any one of embodiments A1 to A49 or a pharmaceutically acceptable salt thereof (including any further embodiment thereof), wherein R^{A} is —C(═O)—OH.
A51. A compound selected from Examples/Compounds 1 to 14, or a pharmaceutically acceptable salt thereof (or its free acid form or a pharmaceutically acceptable salt of its free acid form where an example is a salt), or a racemate thereof (including a pharmaceutically acceptable salt thereof of the racemate), or an enantiomer of the racemate (including its pharmaceutically acceptable salt).

In some further embodiments, the invention provides a compound selected from the group consisting of:
(1S,4s)-4-{4-[(1-{[4-(trifluoromethyl)phenyl]carbamoyl}-D-prolyl)amino]-1,3-thiazol-2-yl}cyclohexane-1-carboxylic acid;
(1R,4r)-4-{4-[(1-{[4-(trifluoromethyl)phenyl]carbamoyl}-D-prolyl)amino]-1,3-thiazol-2-yl}cyclohexane-1-carboxylic acid;
(1S,4s)-4-{4-[(1-{[4-(trifluoromethoxy)phenyl]carbamoyl}-D-prolyl)amino]-1,3-thiazol-2-yl}cyclohexane-1-carboxylic acid;
(1R,4r)-4-{4-[(1-{[4-(trifluoromethoxy)phenyl]carbamoyl}-D-prolyl)amino]-1,3-thiazol-2-yl}cyclohexane-1-carboxylic acid;
(3S)-1-{3-fluoro-5-[(1-{[4-(trifluoromethoxy)phenyl]carbamoyl}-D-prolyl)amino]pyridin-2-yl}pyrrolidine-3-carboxylic acid;
(3R)-1-{3-fluoro-5-[(1-{[4-(trifluoromethoxy)phenyl]carbamoyl}-D-prolyl)amino]pyridin-2-yl}pyrrolidine-3-carboxylic acid;
(1S,3R)-3-{3-Fluoro-5-[(1-{[4-(trifluoromethyl)phenyl]carbamoyl}-D-prolyl)amino]pyridin-2-yl}cyclopentane-1-carboxylic acid;
(1R,3R)-3-{3-Fluoro-5-[(1-{[4-(trifluoromethyl)phenyl]carbamoyl}-D-prolyl)amino]pyridin-2-yl}cyclopentane-1-carboxylic acid;
(1S,3S)-3-{3-Fluoro-5-[(1-{[4-(trifluoromethyl)phenyl]carbamoyl}-D-prolyl)amino]pyridin-2-yl}cyclopentane-1-carboxylic acid;
(1R,3S)-3-{3-Fluoro-5-[(1-{[4-(trifluoromethyl)phenyl]carbamoyl}-D-prolyl)amino]pyridin-2-yl}cyclopentane-1-carboxylic acid;
(1S,3R)-3-{3-Fluoro-5-[(1-{[4-(trifluoromethoxy)phenyl]carbamoyl}-D-prolyl)amino]pyridin-2-yl}cyclopentane-1-carboxylic acid;
(1R,3R)-3-{3-Fluoro-5-[(1-{[4-(trifluoromethoxy)phenyl]carbamoyl}-D-prolyl)amino]pyridin-2-yl}cyclopentane-1-carboxylic acid;
(1S,3S)-3-{3-Fluoro-5-[(1-{[4-(trifluoromethoxy)phenyl]carbamoyl}-D-prolyl)amino]pyridin-2-yl}cyclopentane-1-carboxylic acid; and
(1R,3S)-3-{3-Fluoro-5-[(1-{[4-(trifluoromethoxy)phenyl]carbamoyl}-D-prolyl)amino]pyridin-2-yl}cyclopentane-1-carboxylic acid, or a pharmaceutically acceptable salt thereof.

Each of the embodiments (including all further embodiments) described herein may be combined with any one or more of other embodiments (including all further embodiments) described herein not inconsistent with the embodiment(s) with which it is combined. In addition, any of the compounds described in the Examples, or pharmaceutically acceptable salts thereof, may be claimed individually or grouped together with one or more other compounds of the Examples, or pharmaceutically acceptable salts thereof, for any of the embodiment(s) described herein.

B1. A pharmaceutical composition comprising a compound of any one of embodiments A1 to A51 or a pharmaceutically acceptable salt thereof including the further embodiments described herein and a pharmaceutically acceptable excipient.
C₁. A method for treating or preventing a condition, disease, or disorder in a patient comprising administering to the patient a compound of any one of embodiments A1 to A51 or a pharmaceutically acceptable salt thereof including the further embodiments described herein, wherein the condition, disease, or disorder is selected from the group consisting of diabetes [e.g. Type 1 diabetes mellitus (T1D), Type 2 diabetes mellitus (T2DM), including pre-diabetes], idiopathic T1D (Type 1b), latent autoimmune diabetes in adults (LADA), early-onset T2DM (EOD), youth-onset atypical diabetes (YOAD), maturity onset diabetes of the young (MODY), malnutrition-related diabetes, gestational diabetes, hyperglycemia, insulin resistance, hepatic insulin resistance, impaired glucose tolerance, diabetic neuropathy, diabetic nephropathy, kidney disease [e.g., acute kidney disorder, tubular dysfunction, proinflammatory changes to the proximal tubules, or chronic kidney disease (CKD)], diabetic retinopathy, adipocyte dysfunction, visceral adipose deposition, sleep apnea [e.g. obstructive sleep apnea (OSA)], obesity (including hypothalamic obesity and monogenic obesity) and related comorbidities (e.g., osteoarthritis and urine incontinence), eating disorders (including binge eating syndrome, bulimia nervosa, and syndromic obesity such as Prader-Willi and Bardet-Biedl syndromes), weight gain such as weight gain caused by use of other agents (e.g., caused by use of steroids and/or antipsychotics, or caused by treatment of depression, or caused by use of agents on cognitive function), overweight, excessive sugar craving, dyslipidemia [including hyperlipidemia, hypertriglyceridemia, increased total cholesterol, high LDL (low-density lipoprotein) cholesterol, and low HDL (high-density lipoprotein) cholesterol], hyperinsulinemia, nonalcoholic fatty liver disease [NAFLD, including related diseases such as steatosis, nonalcoholic steatohepatitis (NASH), fibrosis, cirrhosis, and hepatocellular carcinoma], cardiovascular disease, atherosclerosis (including coronary artery disease), peripheral vascular disease, hypertension, endothelial dysfunction, impaired vascular compliance, heart failure [e.g. congestive heart failure, heart failure with preserved ejection fraction (HFpEF), heart failure with reduced ejection fraction (HfrEF)], myocardial infarction (e.g. necrosis and apoptosis), stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, osteoarthritis, Parkinson's disease, left ventricular hypertrophy, peripheral arterial disease (PAD), macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, polycystic ovary syndrome (PCOS), and addiction (e.g., addition to alcohol, nicotine, and/or drug); or a method for weight management (e.g. chronic weight management) of a human comprising administering to the human a compound of any one of embodiments A1 to A51 or a pharmaceutically acceptable salt thereof including the further embodiments described herein.

As used herein, treating diabetes (e.g. T2DM) in a diabetic patient (e.g. a patient with T2DM) includes, among other things, improving glycemic control.

C₂. A method of embodiment C₁, wherein the condition, disease, or disorder is selected from the group consisting of obesity, weight gain, T2DM, Heart Failure (e.g. HFpEF and HFrEF); CKD; NAFLD, NASH, atherosclerosis, PAD, obstructive sleep apnea, diabetic retinopathy, and diabetic neuropathy.
C₃. A method of embodiment C₁, wherein the method is for preventing weight gain.
C₄. A method of embodiment C₁, wherein the method is for preventing obesity.
C₅. A method of embodiment C₁, wherein the method is for treating obesity.
C₆. A method of embodiment C₁, wherein the method is for weight management, for example chronic weight management, of a human. In some further embodiments, the human is obese or overweight when the weight management (e.g. chronic weight management) is initiated; and in such a situation, the weight management (e.g. chronic weight management) is also a method for treating obesity or overweight. In some further embodiments, the human is obese when the weight management (e.g. chronic weight management) treatment is initiated; and in such a situation, the weight management (e.g. chronic weight management) is also a method for treating obesity.
D1. Use of a compound of any one of embodiments A1 to A51 or a pharmaceutically acceptable salt thereof including the further embodiments described herein for treating or preventing a condition, disease, or disorder, or use of a compound in manufacturing a medicament for treating or preventing a condition, disease, or disorder, wherein the condition, disease, or disorder is selected from the group consisting of diabetes [e.g. Type 1 diabetes mellitus (T1D), Type 2 diabetes mellitus (T2DM), including pre-diabetes], idiopathic T1D (Type 1b), latent autoimmune diabetes in adults (LADA), early-onset T2DM (EOD), youth-onset atypical diabetes (YOAD), maturity onset diabetes of the young (MODY), malnutrition-related diabetes, gestational diabetes, hyperglycemia, insulin resistance, hepatic insulin resistance, impaired glucose tolerance, diabetic neuropathy, diabetic nephropathy, kidney disease [e.g., acute kidney disorder, tubular dysfunction, proinflammatory changes to the proximal tubules, or chronic kidney disease (CKD)], diabetic retinopathy, adipocyte dysfunction, visceral adipose deposition, sleep apnea [e.g. obstructive sleep apnea (OSA)], obesity (including hypothalamic obesity and monogenic obesity) and related comorbidities (e.g., osteoarthritis and urine incontinence), eating disorders (including binge eating syndrome, bulimia nervosa, and syndromic obesity such as Prader-Willi and Bardet-Biedl syndromes), weight gain such as weight gain caused by use of other agents (e.g., caused by use of steroids and/or antipsychotics, or caused by treatment of depression, or caused by use of agents on cognitive function), overweight, excessive sugar craving, dyslipidemia [including hyperlipidemia, hypertriglyceridemia, increased total cholesterol, high LDL (low-density lipoprotein) cholesterol, and low HDL (high-density lipoprotein) cholesterol], hyperinsulinemia, nonalcoholic fatty liver disease [NAFLD, including related diseases such as steatosis, nonalcoholic steatohepatitis (NASH), fibrosis, cirrhosis, and hepatocellular carcinoma], cardiovascular disease, atherosclerosis (including coronary artery disease), peripheral vascular disease, hypertension, endothelial dysfunction, impaired vascular compliance, heart failure [e.g. congestive heart failure, heart failure with preserved ejection fraction (HFpEF), heart failure with reduced ejection fraction (HFrEF)], myocardial infarction (e.g. necrosis and apoptosis), stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, osteoarthritis, Parkinson's disease, left ventricular hypertrophy, peripheral arterial disease (PAD), macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, polycystic ovary syndrome (PCOS), and addiction (e.g., addition to alcohol, nicotine, and/or drug); or use of a compound of any one of embodiments A1 to A51 or a pharmaceutically acceptable salt thereof including the further embodiments described herein for weight management (e.g. chronic weight management); or use of a compound of any one of embodiments A1 to A51 or a pharmaceutically acceptable salt thereof including the further embodiments described herein in manufacturing a medicament for weight management (e.g. chronic weight management).
D2. A use of embodiment D1, wherein the condition, disease, or disorder is selected from the group consisting of obesity, weight gain, T2DM, Heart Failure (e.g. HFpEF and HFrEF); CKD; NAFLD, NASH, atherosclerosis, PAD, obstructive sleep apnea, diabetic retinopathy, and diabetic neuropathy.
D3. A use of embodiment D1, wherein the use of a compound of any one of embodiments A1 to A51 or a pharmaceutically acceptable salt thereof including the further embodiments described herein is for preventing weight gain.
D4. A use of embodiment D1, wherein the use of a compound of any one of embodiments A1 to A51 or a pharmaceutically acceptable salt thereof including the further embodiments described herein is in manufacturing a medicament for preventing weight gain.
D5. A use of embodiment D1, wherein the use of a compound of any one of embodiments A1 to A51 or a pharmaceutically acceptable salt thereof including the further embodiments described herein is for treating obesity.
D6. A use of embodiment D1, wherein the use of a compound of any one of embodiments A1 to A51 or a pharmaceutically acceptable salt thereof including the further embodiments described herein is in manufacturing a medicament for obesity.
D7. A use of embodiment D1, wherein the use of a compound of any one of embodiments A1 to A51 or a pharmaceutically acceptable salt thereof including the further embodiments described herein is in manufacturing a medicament for weight management, for example chronic weight management of a human. In some further embodiments, the human is obese or overweight when the weight management (e.g. chronic weight management) is initiated; and in such a situation, the weight management is also a method for treating obesity or overweight. In some further embodiments, the human is obese when the weight management (e.g. chronic weight management) treatment is initiated; and in such a situation, the weight management is also a method for treating obesity.
E1. A compound of any one of embodiments A1 to A51 or a pharmaceutically acceptable salt thereof including the further embodiments described herein for use in a method for treating or preventing a condition, disease, or disorder in a patient, wherein the condition, disease, or disorder is selected from the group consisting of diabetes [e.g. Type 1 diabetes mellitus (T1D), Type 2 diabetes mellitus (T2DM), including pre-diabetes], idiopathic T1D (Type 1b), latent autoimmune diabetes in adults (LADA), early-onset T2DM (EOD), youth-onset atypical diabetes (YOAD), maturity onset diabetes of the young (MODY), malnutrition-related diabetes, gestational diabetes, hyperglycemia, insulin resistance, hepatic insulin resistance, impaired glucose tolerance, diabetic neuropathy, diabetic nephropathy, kidney disease [e.g., acute kidney disorder, tubular dysfunction, proinflammatory changes to the proximal tubules, or chronic kidney disease (CKD)], diabetic retinopathy, adipocyte dysfunction, visceral adipose deposition, sleep apnea [e.g. obstructive sleep apnea (OSA)], obesity (including hypothalamic obesity and monogenic obesity) and related comorbidities (e.g., osteoarthritis and urine incontinence), eating disorders (including binge eating syndrome, bulimia nervosa, and syndromic obesity such as Prader-Willi and Bardet-Biedl syndromes), weight gain such as weight gain caused by use of other agents (e.g., caused by use of steroids and/or antipsychotics, or caused by treatment of depression, or caused by use of agents on cognitive function), overweight, excessive sugar craving, dyslipidemia [including hyperlipidemia, hypertriglyceridemia, increased total cholesterol, high LDL (low-density lipoprotein) cholesterol, and low HDL (high-density lipoprotein) cholesterol], hyperinsulinemia, nonalcoholic fatty liver disease [NAFLD, including related diseases such as steatosis, nonalcoholic steatohepatitis (NASH), fibrosis, cirrhosis, and hepatocellular carcinoma], cardiovascular disease, atherosclerosis (including coronary artery disease), peripheral vascular disease, hypertension, endothelial dysfunction, impaired vascular compliance, heart failure [e.g. congestive heart failure, heart failure with preserved ejection fraction (HFpEF), heart failure with reduced ejection fraction (HFrEF)], myocardial infarction (e.g. necrosis and apoptosis), stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, osteoarthritis, Parkinson's disease, left ventricular hypertrophy, peripheral arterial disease (PAD), macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, polycystic ovary syndrome (PCOS), and addiction (e.g., addition to alcohol, nicotine, and/or drug); or is a compound of any one of embodiments A1 to A51 or a pharmaceutically acceptable salt thereof including the further embodiments described herein for use in a method for weight management (e.g. chronic weight management).
E2. A compound for use of embodiment E1, wherein the condition, disease, or disorder is selected from the group consisting of obesity, weight gain, T2DM, Heart Failure (e.g. HFpEF and HFrEF); CKD; NAFLD, NASH, atherosclerosis, PAD, obstructive sleep apnea, diabetic retinopathy, and diabetic neuropathy.
E3. A compound for use of embodiment E1, wherein a compound of any one of embodiments A1 to A51 or a pharmaceutically acceptable salt thereof including the further embodiments described herein is for use in a method for preventing weight gain.
E4. A compound for use of embodiment E1, wherein a compound of any one of embodiments A1 to A51 or a pharmaceutically acceptable salt thereof including the further embodiments described herein is for use in a method for treating obesity.
E5. A compound for use of embodiment E1, wherein a compound of any one of embodiments A1 to A51 or a pharmaceutically acceptable salt thereof including the further embodiments described herein is for use in a method for weight management (e.g. chronic weight management).

In some further embodiments, the human is obese or overweight when the weight management (e.g. chronic weight management) is initiated; and in such a situation, the weight management is also a method for treating obesity or overweight. In some further embodiments, the human is obese when the weight management (e.g. chronic weight management) treatment is initiated; and in such a situation, the weight management is also a method for treating obesity.

F1. A method for modulating (e.g. antagonizing) a GIPR (either in vitro or in vivo), comprising contacting (including incubating) the GIPR with a compound of any one of embodiments A1 to A51 or a pharmaceutically acceptable salt thereof including the further embodiments described herein.
F2. A method of embodiment F1, wherein said modulating is antagonizing.

It is to be understood that this invention is not limited to specific synthetic methods of preparation described in the schemes herein. It is also to be understood that the terminology used herein is for the purpose of describing particular embodiments only and is not intended to be limiting. In this specification and in the claims that follow, reference will be made to a number of terms that shall be defined to have the following meanings:

As used herein in the specification, “a” or “an” may mean one or more. As used herein in the claim(s), when used in conjunction with the word “comprising”, the words “a” or “an” may mean one or more than one. As used herein “another” may mean at least a second or more.

The term “about” refers to a relative term denoting an approximation of plus or minus 10% of the nominal value to which it refers, in one embodiment, to plus or minus 5%, in another embodiment, to plus or minus 2%. For the field of this disclosure, this level of approximation is appropriate unless the value is specifically stated to require a tighter range.

“Compound” when used herein includes any pharmaceutically acceptable derivative or variation, including conformational isomers (e.g., cis and trans isomers) and all optical isomers (e.g., enantiomers and diastereomers), racemic, diastereomeric and other mixtures of such isomers, as well as solvates, hydrates, isomorphs, polymorphs, tautomers, esters, salt forms, and prodrugs.

As used herein, a wavy line,

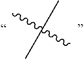

denotes a point of attachment of a substituent to another group.

When a bond to a substituent is shown to cross a bond connecting two atoms in a ring, then such substituent may be bonded to any of the ring-forming atoms in the ring that are substitutable (i.e., bonded to one or more hydrogen atoms). For example, as shown in Formula a-101 below, an R² may be bonded to any one of the substitutable ring-forming carbon or nitrogen atoms of the ring having the variable n1, each of which bears a hydrogen atom (for example, a ring-forming carbon atom of CH₂ or a ring-forming nitrogen atom when A¹ is NH). For another example, as shown in Formula a-102 below, an R^{cy1} may be bonded to any one of the substitutable ring-forming atoms of the ring having the variable V⁴, each of which bears a hydrogen atom (for example, a ring-forming carbon atom of CH₂; the ring-forming carbon atom of the CH moiety that is bonded to R^{A}; or ring-forming carbon atom of V⁴ when V⁴ is CH).

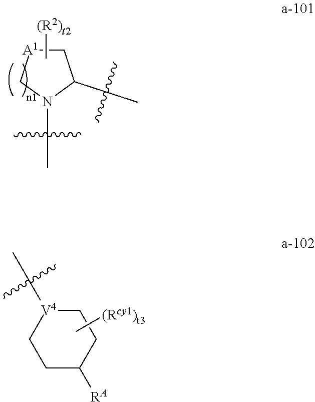

The term “alkyl” means an acyclic, saturated aliphatic hydrocarbon group which may be straight/linear or branched. Examples of such groups include, but are not limited to, methyl, ethyl, n-propyl, isopropyl, butyl, sec-butyl, isobutyl and tert-butyl. The carbon atom content of alkyl and various other hydrocarbon-containing moieties is indicated by a prefix designating a lower and upper number of carbon atoms in the moiety, that is, the prefix Cᵢ₋ⱼ indicates a moiety of the integer “i” to the integer “j” carbon atoms, inclusive. Thus, for example, C₁₋₃ alkyl refers to alkyl of one to eight carbon atoms, inclusive; for another example, C₁₋₆ alkyl refers to alkyl of one to six carbon atoms, inclusive; for yet another example, C₁₋₄ alkyl refers to alkyl of one to four carbon atoms, inclusive. Representative examples of C₁₋₄ alkyl include methyl, ethyl, n-propyl, isopropyl, n-butyl, sec-butyl, isobutyl, and tert-butyl. For another example, C₁₋₂ alkyl refers to alkyl of one to two carbon atoms, inclusive (i.e., methyl or ethyl). The alkyl group optionally can be substituted by 1 or more (e.g., 1 to 5) suitable substituents, when so specified.

At various places in the present specification, substituents of compounds of the invention are disclosed in groups or in ranges. It is specifically intended that the invention include each and every individual sub-combination of the members of such groups and ranges. For example, the term “C₁₋₄ alkyl” is specifically intended to include C₁ alkyl (methyl), C₂ alkyl (ethyl), C₃ alkyl, and C₄ alkyl. For another example, the term “4- to 7-membered heterocycloalkyl” is specifically intended to include any 4-, 5-, 6-, or 7-membered heterocycloalkyl group. For yet another example, the term “C₃₋₆ cycloalkyl” is specifically intended to include any saturated or unsaturated, non-aromatic, monocyclic or polycyclic (such as bicyclic) hydrocarbon rings of 3, 4, 5, or 6 ring-forming carbon atoms.

As used herein, the term “n-membered”, where n is an integer, typically describes the number of ring-forming atoms in a moiety where the number of ring-forming atoms is n. For example, piperidinyl is an example of a 6-membered heterocycloalkyl ring and pyrrolindinyl is an example of a 5-membered heterocycloalkyl group.

As used herein, the term “alkoxy” or “alkyloxy” refers to an —O-alkyl group. For example, the term “C₁₋₄ alkoxy” or “C₁₋₄ alkyloxy” refers to an —O—(C₁₋₄ alkyl) group; For another example, the term “C₁₋₂ alkoxy” or “C₁₋₂ alkyloxy” refers to an —O—(C₁₋₂ alkyl) group. Examples of alkoxy include methoxy, ethoxy, propoxy (e.g., n-propoxy and isopropoxy), tert-butoxy, and the like. The alkoxy or alkyloxy group optionally can be substituted by 1 or more (e.g., 1 to 5) suitable substituents when so specified.

The term “halo” or “halogen” as used herein, means —F, —Cl, —Br, or —I.

As used herein, the term “haloalkyl” refers to an alkyl group having one or more halogen substituents (up to perhaloalkyl, i.e., every hydrogen atom of the alkyl group has been replaced by a halogen atom). For example, the term “C₁₋₄ haloalkyl” refers to a C₁₋₄ alkyl group having one or more halogen substituents (up to perhaloalkyl, i.e., every hydrogen atom of the alkyl group has been replaced by a halogen atom); and the term “C₁₋₂ haloalkyl” refers to a C₁₋₂ alkyl group (i.e., methyl or ethyl) having one or more halogen substituents (up to perhaloalkyl, i.e., every hydrogen atom of the alkyl group has been replaced by a halogen atom). Examples of haloalkyl groups include —CF₃, —CHF₂, —CH₂F, —CH₂CF₃, —C₂F5, —CH₂Cl and the like. “Fluoroalkyl” as used herein means an alkyl as defined herein substituted with one or more fluoro (—F) substituents (up to perfluoroalkyl, i.e., every hydrogen atom of the alkyl group has been replaced by a fluorine atom). The term “C₁₋₄ fluoroalkyl” refers to a C₁₋₄ alkyl group (e.g., methyl or ethyl) having one or more fluorine substituents (up to perfluoroalkyl, i.e., every hydrogen atom of the alkyl group has been replaced by a fluorine atom); the term “C₁₋₂ fluoroalkyl” refers to a C₁₋₂ alkyl group (i.e., methyl or ethyl) having one or more fluorine substituents (up to perfluoroalkyl, i.e., every hydrogen atom of the alkyl group has been replaced by a fluorine atom); and the term “C₁ fluoroalkyl” refers to methyl having 1, 2, or 3 fluorine substituents. Examples of C₁ fluoroalkyl include fluoromethyl, difluoromethyl and trifluoromethyl; some examples of C₂ fluoroalkyl include 1-fluoroethyl, 2-fluoroethyl, 2,2-difluoroethyl, 1,2-difluoroethyl, 2,2,2-trifluoroethyl, 1,1,2-trifluoroethyl, and the like.

As used here, the term “haloalkoxy” refers to an —O-haloalkyl group. For example, the term “C₁0.4 haloalkoxy” refers to an —O—(C₁₋₄ haloalkyl) group; and the term “C₁₋₂ haloalkoxy” refers to an —O—(C₁₋₂ haloalkyl) group. For yet another example, the term “C₁ haloalkoxy” refers to a methoxy group having one, two, or three halogen substituents. An example of haloalkoxy is —OCF₃ or —OCHF₂.

As used here, the term “fluoroalkoxy” refers to an —O-fluoroalkyl group. For example, the term “C₁₋₄ fluoroalkoxy” refers to an —O—(C₁₋₄ fluoroalkyl) group; the term “C₁₋₂ fluoroalkoxy” refers to an —O—(C₁₋₂ fluoroalkyl) group; and the term “C₁ fluoroalkoxy” refers to an —O—(C₁ fluoroalkyl) group. Examples of C₁ fluoroalkoxy include —O—CH₂F, —O—CHF₂, and —O—CF₃. Some examples of C₂ fluoroalkoxy include —O—CH₂CHF₂, —O—CH₂—CHF₂, —O—CH₂CF₃, —O—CF₂CH₃, and —O—CF₂CF₃.

As used herein, the term “hydroxylalkyl” or “hydroxyalkyl” refers to an alkyl group having one or more (e.g., 1, 2, or 3) OH substituents. The term “C₁₋₄ hydroxylalkyl” or “C₁₋₄ hydroxyalkyl” refers to a C₁₋₄ alkyl group having one or more (e.g., 1, 2, or 3) OH substituents; and the term “C₁₋₂ hydroxylalkyl” or “C₁₋₂ hydroxyalkyl” refers to a C₁₋₂ alkyl group having one or more (e.g., 1, 2, or 3) OH substituents. An example of hydroxylalkyl is —CH₂OH or —CH₂CH₂OH.

As used herein, the term “cyanoalkyl” refers to an alkyl group having one or more (e.g., 1, 2, or 3) —CN (i.e. —C≡N or cyano) substituents. For example, The term “C₁₋₄ cyanoalkyl” refers to a C₁₋₄ alkyl group having one or more (e.g., 1, 2, or 3) —CN substituents. An example of cyanoalkyl is —CH₂—CN or —CH₂CH₂—CN.

As used herein, the term “alkenyl” refers to aliphatic hydrocarbons having at least one carbon-carbon double bond, including straight chains and branched chains having at least one carbon-carbon double bond. In some embodiments, the alkenyl group has 2 to 20 carbon atoms, 2 to 10 carbon atoms, 2 to 6 carbon atoms, 3 to 6 carbon atoms, or 2 to 4 carbon atoms. For example, as used herein, the term “C₂₋₈ alkenyl” refers to straight or branched chain unsaturated radicals (having at least one carbon-carbon double bond) of 2 to 8 carbon atoms; the term “C₃₋₆ alkenyl” refers to straight or branched chain unsaturated radicals (having at least one carbon-carbon double bond) of 3 to 6 carbon atoms; and the term “C₃₋₄ alkenyl” refers to straight or branched chain unsaturated radicals (having at least one carbon-carbon double bond) of 3 to 4 carbon atoms. Examples of “C₃₋₆ alkenyl” include, but are not limited to, prop-2-en-1-yl, prop-1-en-2-yl, but-2-en-1-yl, but-2-en-2-yl, 2-methylbut-2-en-1-yl, and the like. An alkenyl group optionally can be substituted by one or more (e.g. 1 to 5) suitable substituents. When the compounds of Formula I contain an alkenyl group, the alkenyl group may exist as the pure E form, the pure Z form, or any mixture thereof when applicable.

As used herein, the term “cycloalkyl” refers to saturated or unsaturated, non-aromatic, monocyclic or polycyclic (such as bicyclic) hydrocarbon rings (e.g., monocyclics such as cyclopropyl, cyclobutyl, cyclopentyl, cyclohexyl, cycloheptyl, cyclooctyl, cyclononyl, or bicyclics including spiro, fused, or bridged systems (such as bicyclo[1.1.1]pentanyl, bicyclo[2.2.1]heptanyl, bicyclo[3.2.1]octanyl or bicyclo[5.2.0]nonanyl, decahydronaphthalenyl, etc.). The cycloalkyl group has 3 to 15 (e.g., 3 to 14, 3 to 10, 3 to 6, 3 to 4, or 4 to 6) carbon atoms. In some embodiments the cycloalkyl may optionally contain one, two, or more non-cumulative non-aromatic double or triple bonds and/or one to three oxo groups. In some embodiments, the bicycloalkyl group has 6 to 14 carbon atoms. The term “C₃₋₆ cycloalkyl” as used herein, means a saturated or unsaturated (but non-aromatic) cyclic hydrocarbon group containing from 3 to 6 carbons. The term “C₃₋₄ cycloalkyl” as used herein, means a saturated cyclic hydrocarbon group containing from 3 to 4 carbons. Examples of C₃₋₄ cycloalkyl include cyclopropyl and cyclobutyl. Also included in the definition of cycloalkyl are moieties that have one or more aromatic rings (including aryl and heteroaryl) fused to the cycloalkyl ring, for example, benzo or pyridinyl derivatives of cyclopentane (a 5-membered cycloalkyl), cyclopentene, cyclohexane (a 6-membered cycloalkyl), and the like, for example, 6,7-dihydro-5H-cyclopenta[b]pyridinyl, 5,6,7,8-tetrahydroquinolinyl, or 1 5,6,7,8-tetrahydroisoquinolinyl, each of which includes a 5-membered or 6-membered cycloalkyl moiety that is fused to a heteroaryl ring (i.e. the pyridinyl ring). The cycloalkyl or C₃₋₄ cycloalkyl group optionally can be substituted by 1 or more (e.g., 1 to 5) suitable substituents when so specified.

The term “—C₁₋₄ alkyl-C₃₋₆ cycloalkyl” or “C₃₋₆ cycloalkyl-C₁₋₄ alkyl-” as used herein, means a C₃₋₆ cycloalkyl as defined herein, appended to the parent molecular moiety through a C₃₋₄ alkyl group, as defined herein. The term “C₁₋₄ alkyl-C₃₋₄ cycloalkyl” or “C₃₋₄ cycloalkyl-C₁₋₄ alkyl-” as used herein, means a C₃₋₄ cycloalkyl as defined herein, appended to the parent molecular moiety through a C₃₋₄ alkyl group, as defined herein. Some examples of C₃₋₄ cycloalkyl-C₁₋₄ alkyl-include cyclopropylmethyl, 2-cyclopropylethyl, 2-cyclopropylpropyl, 3-cyclopropylpropyl, cyclobutylmethyl, 2-cyclobutylethyl, 2-cyclobutylpropyl, and 3-cyclobutylpropyl.

The term “C₃₋₆ cycloalkyl-C₁₋₂ alkyl-” or “—C₁₋₂ alkyl-C₃₋₆ cycloalkyl” as used herein, means a C₃₋₆ cycloalkyl as defined herein, appended to the parent molecular moiety through a C₁₋₂ alkyl group, as defined herein. The term “C₃₋₄ cycloalkyl-C₁₋₂ alkyl-” or “—C₁₋₂ alkyl-C₃₄ cycloalkyl” as used herein, means a C₃₋₄ cycloalkyl as defined herein, appended to the parent molecular moiety through a C₁₋₂ alkyl group, as defined herein.

As used herein, the term “heterocycloalkyl” refers to a monocyclic or polycyclic [including 2 or more rings that are fused together, including spiro, fused, or bridged systems, for example, a bicyclic ring system], saturated or unsaturated, non-aromatic 4- to 15-membered ring system (such as a 4- to 14-membered ring system, 4- to 12-membered ring system, 5- to 10-membered ring system, 4- to 7-membered ring system, 4- to 6-membered ring system, or 5- to 6-membered ring system), including 1 to 14 ring-forming carbon atoms and 1 to 10 ring-forming heteroatoms each independently selected from O, S and N (and optionally P or B when present). The heterocycloalkyl group can also optionally contain one or more oxo (i.e., ═O) or thiono (i.e., ═S) groups. For example, the term “4- to 7-membered heterocycloalkyl” refers to a monocyclic or polycyclic, saturated or unsaturated, non-aromatic 4- to 7-membered ring system that comprises one or more ring-forming heteroatoms each independently selected from O, S and N. For another example, the term “5- or 6-membered heterocycloalkyl” refers to a monocyclic or polycyclic, saturated or unsaturated, non-aromatic 5- or 6-membered ring system that comprises one or more ring-forming heteroatoms each independently selected from O, S and N. Also included in the definition of heterocycloalkyl are moieties that have one or more aromatic rings (including aryl and heteroaryl) fused to the heterocycloalkyl ring, for example, isoindolinyl [i.e. a pyrrolidinyl ring (an example of 5-membered heterocycloalkyl) fused to a benzo ring (an example of aryl)]. The heterocycloalkyl group optionally can be substituted by 1 or more (e.g., 1 to 5) suitable substituents, when so specified.

Some examples of 4- to 7-membered heterocycloalkyl include azetidinyl, oxetanyl, tetrahydrofuranyl, imidazolidinyl, pyrrolidinyl, piperidinyl, piperazinyl, oxazolidinyl, thiazolidinyl, pyrazolidinyl, thiomorpholinyl, tetrahydrothiazinyl, tetrahydrothiadiazinyl, morpholinyl, tetrahydrodiazinyl, and tetrahydropyranyl (also known as oxanyl). Some further examples of 4- to 7-heterocycloalkyl include tetrahydrofuran-2-yl, tetrahydrofuran-3-yl, tetrahydropyranyl (e.g., tetrahydro-2H-pyran-4-yl), imidazolidin-1-yl, imidazolidin-2-yl, imidazolidin-4-yl, pyrrolidin-1-yl, pyrrolidin-2-yl, pyrrolidin-3-yl, piperidin-1-yl, piperidin-2-yl, piperidin-3-yl, piperidin-4-yl, piperazin-1-yl, piperazin-2-yl, 1,3-oxazolidin-3-yl, 1,4-oxazepan-2-yl, isothiazolidinyl, 1,3-thiazolidin-3-yl, 1,2-pyrazolidin-2-yl, 1,2-tetrahydrothiazin-2-yl, 1,3-thiazinan-3-yl, 1,2-tetrahydrodiazin-2-yl, 1,3-tetrahydrodiazin-1-yl, 1,4-oxazin-4-yl, oxazolidinonyl, 2-oxo-piperidinyl (e.g., 2-oxo-piperidin-1-yl), 2-oxoazepan-3-yl, and the like.

As used herein, the term “heteroaryl” refers to monocyclic or fused-ring polycyclic aromatic heterocyclic groups with one or more heteroatom ring members (ring-forming atoms) each independently selected from O, S and N in at least one ring. The heteroaryl group has 5 to 14 ring-forming atoms, including 1 to 13 carbon atoms, and 1 to 8 heteroatoms selected from O, S, and N. In some embodiments, the heteroaryl group has 5 to 10 ring-forming atoms including one to four heteroatoms. The heteroaryl group can also contain one to three oxo or thiono (i.e., ═S) groups. In some embodiments, the heteroaryl group has 5 to 8 ring-forming atoms including one, two or three heteroatoms. For example, the term “5-membered heteroaryl” refers to a monocyclic heteroaryl group as defined above with 5 ring-forming atoms in the monocyclic heteroaryl ring; the term “6-membered heteroaryl” refers to a monocyclic heteroaryl group as defined above with 6 ring-forming atoms in the monocyclic heteroaryl ring; and the term “5- or 6-membered heteroaryl” refers to a monocyclic heteroaryl group as defined above with 5 or 6 ring-forming atoms in the monocyclic heteroaryl ring. A heteroaryl group optionally can be substituted by 1 or more (e.g., 1 to 5) suitable substituents, when so specified. Examples of monocyclic heteroaryls include those with 5 ring-forming atoms including one to three heteroatoms or those with 6 ring-forming atoms including one, two or three nitrogen heteroatoms. Examples of fused bicyclic heteroaryls include two fused 5- and/or 6-membered monocyclic rings including one to four heteroatoms.

Some examples of heteroaryl groups include pyridinyl (e.g., pyridin-2-yl, pyridin-3-yl, pyridine-4-yl), pyrazinyl, pyrimidinyl (e.g., pyrimidin-2-yl, pyrimidin-4-yl, or pyrimidin-5-yl), pyridazinyl (e.g., pyridazin-3-yl, or pyridazin-4-yl), thienyl, furyl, imidazolyl (e.g., 1H-imidazol-4-yl), pyrrolyl, oxazolyl (e.g., 1,3-oxazolyl, 1,2-oxazolyl), thiazolyl (e.g., 1,2-thiazolyl, 1,3-thiazolyl), pyrazolyl (e.g., pyrazol-1-yl, pyrazol-3-yl, pyrazol-4-yl), tetrazolyl (e.g., 2H-tetrazol-5-yl), triazolyl (e.g., 1,2,3-triazolyl, 1,2,4-triazolyl), oxadiazolyl (e.g., 1,2,3-oxadiazolyl, 1,2,4-oxadiazolyl or 1,3,4-oxadiazolyl), thiadiazolyl (e.g., 1,3,4-thiadiazolyl, or 1,2,4-thiadiazolyl), quinolyl, isoquinolyl, benzothienyl, benzofuryl, indolyl, benzothiazolyl, 1,2-benzoxazolyl, 1H-imidazo[4,5-c]pyridinyl, imidazo[1,2-a]pyridinyl, 1H-pyrrolo[3,2-c]pyridinyl, imidazo[1,2-a]pyrazinyl, imidazo[2,1-c][1,2,4]triazinyl, imidazo[1,5-a]pyrazinyl, imidazo[1,2-a]pyrimidinyl, 1H-indazolyl, 9H-purinyl, imidazo[1,2-a]pyrimidinyl, [1,2,4]triazolo[1,5-a]pyridinyl, [1,2,4]triazolo[1,5-a]pyrimidinyl, [1,2,4]triazolo[4,3-b]pyridazinyl, isoxazolo[5,4-c]pyridazinyl, isoxazolo[3,4-c]pyridazinyl, pyrazolo[1,5-a]pyrimidinyl, 6,7-dihydro-5H-pyrrolo[1,2-b][1,2,4]triazolyl, pyridone, pyrimidone, pyrazinone, pyrimidinone, 1H-imidazol-2(3H)-one, 1H-pyrrole-2,5-dione, 3-oxo-2H-pyridazinyl, 1H-2-oxo-pyrimidinyl, 1H-2-oxo-pyridinyl, 2,4(1H,3H)-dioxo-pyrimidinyl, 1H-2-oxo-pyrazinyl, and the like.

As used herein, the term “carboxylic acid bioisostere group” in R^{A} refers to a moiety replacing —C(═O)—OH of R^{A} in a compound of Formula I [including e.g. Formula Ia, II, etc.] having —C(═O)—OH as R^{A} with which would result in another compound of Formula I that would exhibit broadly similar biological activity to the corresponding compound of Formula I wherein R^{A} is —C(═O)—OH (and wherein other variables are the same for both compounds). Carboxylic acid bioisostere groups are known to those skilled in the art. See e.g. K. Bredael et. al, “Carboxylic Acid Bioisosteres in Medicinal Chemistry: Synthesis and Properties”, Journal of Chemistry, vol. 2022, Article ID 2164558, 21 pages, 2022; and Ballatore C, et. al, “Carboxylic acid (bio)isosteres in drug design,” ChemMedChem. 2013 March; 8(3):385-95. In some embodiments, the “carboxylic acid bioisostere group” in R^{A} is an aromatic heterocycle, such as 1H-tetrazol-5-yl, 3-hydroxyisoxazol-5-yl, 5(4H)-oxo-1,2,4-oxadiazol-3-yl-, 5(4H)-oxo-1,2,4-thiadiazol-3-yl-, 2-thioxo-1,3,4-oxadiazol-5-yl-, 4H-1,2,4-triazol-3-yl-, 1H-imidazol-5-yl, 4-hydroxy-1,2,5-oxadiazol-3-yl, 1-hydroxypyrazol-5-yl, or 3-hydroxy-1H-pyrazol-1-yl-. Additional carboxylic acid bioisostere groups include hydroxamic acid —C(═O)—NH(OH), trifluoromethylketone —C(═O)CF₃, 2,2,2-trifluoroethan-1-ol-CH(OH)CF₃, acyl sulfonamide —C(═O)—NH—S(═O)₂—R¹⁰⁰, or sulfonylurea —NH—C(═O)—NH—S(═O)₂—R²⁰⁰, wherein R¹⁰⁰ is C₁₋₆ alkyl or phenyl and where the phenyl is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy; and R²⁰⁰ is aryl (e.g. phenyl) or heteroaryl (e.g. 5- or 6-membered heteroaryl) optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy. In some embodiments, the “carboxylic acid bioisostere group” in R^{A} is selected from 1H-tetrazol-5-yl, 3-hydroxyisoxazol-5-yl, 5(4H)-oxo-1,2,4-oxadiazol-3-yl-, 5(4H)-oxo-1,2,4-thiadiazol-3-yl-, 2-thioxo-1,3,4-oxadiazol-5-yl-, 4H-1,2,4-triazol-3-yl-, 4-hydroxy-1,2,5-oxadiazol-3-yl, 1-hydroxypyrazol-5-yl, and 3-hydroxy-1H-pyrazol-1-yl-. In some other embodiments, the “carboxylic acid bioisostere group” in R^{A} is —C(═)—NH—S(═O)₂—R¹⁰⁰ wherein R¹⁰⁰ is C₁₋₆ alkyl or phenyl and where the phenyl is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy.

As used herein, the compound of Formula I as described herein includes optional substitutions and variables. It is understood that the normal valency of each of the designated (optionally substituted) atom or moiety is not exceeded, and that any of the optional substitution results in a stable compound. It is also understood that combinations of optional substituents and/or variables are permissible only if such combinations result in a stable compound.

As used herein, when a group is described to be optionally substituted, it means that the group can be either unsubstituted or substituted with one or more substitutents as specified.

As used herein, unless otherwise specified, the point of attachment of a substituent can be from any suitable position of the substituent. For example, piperidinyl can be piperidin-1-yl (attached through the N atom of the piperidinyl), piperidin-2-yl (attached through the C atom at the 2-position of the piperidinyl), piperidin-3-yl (attached through the C atom at the 3-position of the piperidinyl), or piperidin-4-yl (attached through the C atom at the 4-position of the piperidinyl). For another example, propanyl (or propyl) can be propan-1-yl (or 1-propyl) or propan-2-yl (or 2-propyl).

As used herein, the point of attachment of a substituent can be specified to indicate the position where the substituent is attached to another moiety. For example, “—C₁₋₄ alkyl-(C₃₋₄ cycloalkyl)” or “(C₃₋₄ cycloalkyl)—C₁₋₄ alkyl-” means the point of attachment occurs at the “C₁₋₄ alkyl” part of the “—C₁₋₄ alkyl-(C₃₋₄ cycloalkyl)” or “(C₃₋₄ cycloalkyl)—C₁₋₄ alkyl-.”

When a substituted or optionally substituted moiety is described without indicating the atom via which such moiety is bonded to a substituent, then the substituent may be bonded via any appropriate atom in such moiety. For example in a substituted “—C₁₋₄ alkyl-(C₃₋₄ cycloalkyl)” or “(C₃₋₄ cycloalkyl)—C₁₋₄ alkyl-”, a substituent on the cycloalkylalkyl [i.e., “—C₁₋₄ alkyl-(C₃₋₄ cycloalkyl)” or “(C₃₋₄ cycloalkyl)—C₁₋₄ alkyl-” ] can be bonded to any carbon atom on the alkyl part or on the cycloalkyl part of the cycloalkylalkyl. Combinations of substituents and/or variables are permissible only if such combinations result in stable compounds.

As used herein, the term “adjacent” in describing the relative positions of two substituent groups on a ring structure refers to two substituent groups that are respectively attached to two ring-forming atoms of the same ring, wherein the two ring-forming atoms are directly connected through a chemical bond. For example, in the following structure:

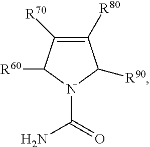

either of R⁶⁰ and R⁸⁰ is an adjacent group of R⁷⁰, but R⁹⁰ is not an adjacent group of R⁷⁰.

“Mammals” refers to warm-blooded vertebrate animals characterized by the secretion of milk by females for the nourishment of the young, such as guinea pigs, mice, rats, gerbils, cats, rabbits, dogs, cattle, goats, sheep, horses, monkeys, chimpanzees, and humans.

The term “pharmaceutically acceptable” means the substance (e.g., the compounds of the invention) and any salt thereof, or composition containing the substance or salt of the invention that is suitable for administration to a patient.

As used herein, the expressions “reaction-inert solvent” and “inert solvent” refer to a solvent or a mixture thereof which does not interact with starting materials, reagents, intermediates or products in a manner which adversely affects the yield of the desired product.

As used herein, the term “selectivity” or “selective” refers to a greater effect of a compound in a first assay, compared to the effect of the same compound in a second assay. For example, in “gut-selective” compounds, the first assay is for the half-life of the compound in the intestine and the second assay is for the half-life of the compound in the liver.

“Therapeutically effective amount” means an amount of a compound of the present invention that (i) treats or prevents the particular disease, condition, or disorder; (ii) attenuates, ameliorates, or eliminates one or more symptoms of the particular disease, condition, or disorder; or (iii) prevents or delays the onset of one or more symptoms of the particular disease, condition, or disorder described herein.

The term “treating”, “treat”, or “treatment” as used herein embraces both preventative, i.e., prophylactic, and palliative treatment, including reversing, relieving, alleviating, or slowing the progression of the disease (or disorder or condition) or any tissue damage associated with one or more symptoms of the disease (or disorder or condition).

As used herein, the term “contacting” refers to the bringing together of indicated moieties in an in vitro system or an in vivo system. For example, “contacting” GIPR with a compound of the invention includes the administration of a compound of the present invention to a mammal, such as a human, having the GIPR, as well as, for example, introducing a compound of the invention into a sample containing a cellular or purified preparation containing the GIPR.

Every embodiment, Example, or pharmaceutically acceptable salt thereof may be claimed individually or grouped together in any combination with any number of each and every embodiment described herein.

The compound of the invention [a compound of Formula I or a pharmaceutically acceptable salt thereof (also including, e.g., a compound of Formula Ia, II, IIa, III, IIIa, IV, IVa, V, Va, VI, VIa, VII, VIIa, VIII, VIIIa, IX, IXa, X, Xa, XI, or XIa, or a pharmaceutically acceptble salt thereof)] can be used in any of the pharmaceutical compositions, uses, and methods of the invention described herein.

### Pharmaceutical Compositions

The present invention also provides a composition (e.g., a pharmaceutical composition) comprising the compound of the invention. Accordingly, in one embodiment, the invention provides a pharmaceutical composition comprising (a therapeutically effective amount of) the compound of the invention and optionally comprising a pharmaceutically acceptable carrier. In addition to the compounds of the invention, the pharmaceutical composition of the invention may also contain, or be co-administered (e.g. simultaneously, sequentially, together, or separately) with, one or more pharmacological agents of value in treating one or more disease conditions referred to herein. In one further embodiment, the invention provides a pharmaceutical composition comprising (a therapeutically effective amount of) a compound of Formula I or a pharmaceutically acceptable salt thereof, optionally comprising a pharmaceutically acceptable carrier and, optionally, at least one additional medicinal or pharmaceutical agent (such as an anti-diabetic agent or weight management agent). In one embodiment, the additional medicinal or pharmaceutical agent is anti-diabetic agent as described below.

A “pharmaceutical composition” of the invention refers to a mixture of (1) one or more of the compounds of the invention as an active ingredient (e.g. a compound of Formula I or a pharmaceutically acceptable salt, including any solvate, hydrate, solid form, stereoisomer, tautomer, or prodrug) and (2) at least one pharmaceutically acceptable excipient.

The term ‘excipient’ is used herein to describe any ingredient other than the compound(s) of the invention. The choice of excipient will to a large extent depend on factors such as the mode of administration, the effect of the excipient on solubility and stability, and the nature of the dosage form.

As used herein, “excipient” includes any and all solvents, dispersion media, coatings, antibacterial agents, antifungal agents, isotonic agents, absorption delaying agents, carriers, diluents and the like that are physiologically compatible. Examples of excipients include one or more of water, saline, phosphate buffered saline, dextrose, glycerol, ethanol and the like, as well as combinations thereof, and may include isotonic agents, for example, sugars, sodium chloride, or polyalcohols such as mannitol, or sorbitol in the composition. Examples of excipients also include various organic solvents (such as hydrates and solvates). The pharmaceutical compositions may, if desired, contain additional excipients such as flavorings, binders/binding agents, lubricating agents, disintegrants, sweetening or flavoring agents, coloring matters or dyes, and the like. For example, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of excipients, therefore, also include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with additional excipients such as water, ethanol, propylene glycol, glycerin, or combinations thereof.

Examples of excipients also include pharmaceutically acceptable substances such as wetting agents or minor amounts of auxiliary substances such as wetting or emulsifying agents, preservatives, or buffers, which enhance the shelf life or effectiveness of the compound.

The compositions of this invention may be in a variety of forms. These include, for example, liquid, semi-solid and solid dosage forms, such as liquid solutions (e.g., injectable and infusible solutions), dispersions or suspensions, tablets, capsules, pills, powders, liposomes and suppositories. The form depends on the intended mode of administration and therapeutic application.

Some compositions are in the form of injectable or infusible solutions, such as compositions similar to those used for passive immunization of humans with antibodies in general. One mode of administration is parenteral (e.g., intravenous, subcutaneous, intraperitoneal, intramuscular). In another embodiment, the compound is administered by intravenous infusion or injection. In yet another embodiment, the compound is administered by intramuscular or subcutaneous injection.

Oral administration of a solid dosage form may be, for example, presented in discrete units, such as hard or soft capsules, pills, cachets, lozenges, or tablets, each containing a predetermined amount of at least one compound of the invention. In another embodiment, the oral administration may be in a powder or granule form. In another embodiment, the oral dosage form is sub-lingual, such as, for example, a lozenge. In such solid dosage forms, the compounds of the invention are ordinarily combined with one or more adjuvants. Such capsules or tablets may comprise a controlled release formulation. In the case of capsules, tablets, and pills, the dosage forms also may comprise buffering agents or may be prepared with enteric coatings.

In another embodiment, oral administration may be in a liquid dosage form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as one or more of wetting, emulsifying, suspending, flavoring (e.g., sweetening), or perfuming agents.

In another embodiment, the invention comprises a parenteral dosage form. “Parenteral administration” includes, for example, subcutaneous injections, intravenous injections, intraperitoneal injections, intramuscular injections, intrasternal injections, and infusion. Injectable preparations (i.e., sterile injectable aqueous or oleaginous suspensions) may be formulated according to the known art using one or more of suitable dispersing, wetting agents, or suspending agents.

In another embodiment, the invention comprises a topical dosage form. “Topical administration” includes, for example, dermal and transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this invention are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical excipients include alcohol, water, mineral oil, liquid petrolatum, white petrolatum, glycerin, polyethylene glycol and propylene glycol. Penetration enhancers may be incorporated—see, for example, B. C. Finnin and T. M. Morgan, J. Pharm. Sci., vol. 88, pp. 955-958, 1999.

Formulations suitable for topical administration to the eye include, for example, eye drops wherein the compound of this invention is dissolved or suspended in a suitable excipient. A typical formulation suitable for ocular or aural administration may be in the form of drops of a micronized suspension or solution in isotonic, pH-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration, the compounds of the invention are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the invention comprises a rectal dosage form. Such rectal dosage form may be in the form of, for example, a suppository. Cocoa butter is a traditional suppository base, but various alternatives may be used as appropriate.

Other excipients and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; Rowe, Raymond C. Handbook of Pharmaceutical Excipients. Chicago, Pharmaceutical Press, 2005; Stahl, P. Heinrich and Camilli G. Wermuth, Eds. Handbook of Pharmaceutical Salts: Properties, Selection, and Use. New York: Wiley-VCH, 2011; and Brittain, Harry G., Ed. Polymorphism in Pharmaceutical Solids. New York: Informa Healthcare USA, Inc., 2016.

Acceptable excipients are nontoxic to subjects at the dosages and concentrations employed, and may comprise one or more of the following: 1) buffers such as phosphate, citrate, or other organic acids; 2) salts such as sodium chloride; 3) antioxidants such as ascorbic acid or methionine; 4) preservatives such as octadecyldimethylbenzyl ammonium chloride, hexamethonium chloride, benzalkonium chloride, benzethonium chloride, phenol, butyl or benzyl alcohol; 5) alkyl parabens such as methyl or propyl paraben, catechol, resorcinol, cyclohexanol, 3-pentanol, or m-cresol; 6) low molecular weight (less than about 10 residues) polypeptides; 7) proteins such as serum albumin, gelatin, or immunoglobulins; 8) hydrophilic polymers such as polyvinylpyrrolidone; 9) amino acids such as glycine, glutamine, asparagine, histidine, arginine, or lysine; 10) monosaccharides, disaccharides, or other carbohydrates including glucose, mannose, or dextrins; 11) chelating agents such as EDTA; 12) sugars such as sucrose, mannitol, trehalose or sorbitol; 13) salt-forming counter-ions such as sodium, metal complexes (e.g., Zn-protein complexes), or 14) non-ionic surfactants such as polysorbates (e.g., polysorbate 20 or polysorbate 80), poloxamers or polyethylene glycol (PEG).

For oral administration, the compositions may be provided in the form of tablets or capsules containing 0.01, 0.05, 0.1, 0.5, 1.0, 2.5, 5.0, 10.0, 15.0, 25.0, 50.0, 75.0, 100, 125, 150, 175, 200, 250 or 500 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient.

Intravenously, doses may range from about 0.01 to about 10 mg/kg/minute during a constant rate infusion.

Liposome-containing compounds of the invention may be prepared by methods known in the art (See, for example, Chang, H. I.; Yeh, M. K.; Clinical development of liposome-based drugs: formulation, characterization, and therapeutic efficacy; Int J Nanomedicine 2012; 7; 49-60). Particularly useful liposomes may be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

Compounds of the invention may also be entrapped in microcapsules prepared, for example, by coacervation techniques or by interfacial polymerization, for example, hydroxymethylcellulose or gelatin microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy, 20th Ed., Mack Publishing (2000).

Sustained-release preparations may be used. Suitable examples of sustained-release preparations include semi-permeable matrices of solid hydrophobic polymers containing a compound of the invention, which matrices are in the form of shaped articles, e.g., films, or microcapsules. Examples of sustained-release matrices include polyesters, hydrogels (for example, poly(2-hydroxyethyl-methacrylate), or ‘poly(vinylalcohol)), polylactides, copolymers of L-glutamic acid and 7 ethyl-L-glutamate, non-degradable ethylene-vinyl acetate, degradable lactic acid-glycolic acid copolymers such as those used in leuprolide acetate for depot suspension (injectable microspheres composed of lactic acid-glycolic acid copolymer and leuprolide acetate), sucrose acetate isobutyrate, and poly-D-(−)-3-hydroxybutyric acid.

The formulations to be used for intravenous administration must be sterile. This is readily accomplished by, for example, filtration through sterile filtration membranes. Compounds of the invention are generally placed into a container having a sterile access port, for example, an intravenous solution bag or vial having a stopper pierceable by a hypodermic injection needle.

Suitable emulsions may be prepared using commercially available fat emulsions, such as a lipid emulsions comprising soybean oil, a fat emulsion for intravenous administration (e.g., comprising safflower oil, soybean oil, egg phosphatides and glycerin in water), emulsions containing soya bean oil and medium-chain triglycerides, and lipid emulsions of cottonseed oil. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion may comprise fat droplets between 0.1 and 1.0 μm, particularly 0.1 and 0.5 μm, and have a pH in the range of 5.5 to 8.0.

For example, the emulsion compositions may be those prepared by mixing a compound of the invention with a lipid emulsions comprising soybean oil or the components thereof (soybean oil, egg phospholipids, glycerol and water).

Compositions for inhalation or insufflation include solutions and suspensions in pharmaceutically acceptable aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

A drug product intermediate (DPI) is a partly processed material that must undergo further processing steps before it becomes bulk drug product. Compounds of the invention may be formulated into drug product intermediate DPI containing the active ingredient in a higher free energy form than the crystalline form. One reason to use a DPI is to improve oral absorption characteristics due to low solubility, slow dissolution, improved mass transport through the mucus layer adjacent to the epithelial cells, and in some cases, limitations due to biological barriers such as metabolism and transporters. Other reasons may include improved solid state stability and downstream manufacturability. In one embodiment, the drug product intermediate contains a compound of the invention isolated and stabilized in the amorphous state (for example, amorphous solid dispersions (ASDs)). There are many techniques known in the art to manufacture ASD's that produce material suitable for integration into a bulk drug product, for example, spray dried dispersions (SDD's), melt extrudates (often referred to as HME's), co-precipitates, amorphous drug nanoparticles, and nano-adsorbates. In one embodiment amorphous solid dispersions comprise a compound of the invention and a polymer excipient. Other excipients as well as concentrations of said excipients and the compound of the invention are well known in the art and are described in standard textbooks. See, for example, “Amorphous Solid Dispersions Theory and Practice” by Navnit Shah et al.

The pharmaceutical composition may, for example, be in a form suitable for oral administration as a tablet, capsule, pill, powder, sustained release formulation, solution or suspension, for parenteral injection as a sterile solution, suspension or emulsion, for topical administration as an ointment or cream or for rectal administration as a suppository.

Exemplary parenteral administration forms include solutions or suspensions of active compounds in sterile aqueous solutions, for example, aqueous propylene glycol or dextrose solutions. Such dosage forms may be suitably buffered, if desired.

The pharmaceutical composition may be in unit dosage forms suitable for single administration of precise dosages. One of ordinary skill in the art would appreciate that the composition may be formulated in sub-therapeutic dosage such that multiple doses are envisioned.

In one embodiment the composition comprises (a therapeutically effective amount of) a compound of Formula I or a pharmaceutically acceptable salt thereof and a pharmaceutically acceptable excipient.

### Administration and Dosing

The term “treating”, “treat” or “treatment” as used herein embraces both preventative, i.e., prophylactic, and palliative treatment, i.e., relieve, alleviate, or slow the progression of the patient's disease (or condition) or any tissue damage associated with the disease.

As used herein, the terms, “subject, “individual” or “patient,” used interchangeably, refer to any animal, including mammals. Mammals according to the invention include canine, feline, bovine, caprine, equine, ovine, porcine, rodents, lagomorphs, primates, humans and the like, and encompass mammals in utero. In an embodiment, humans are suitable subjects. Human subjects may be of any gender and at any stage of development.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which may include one or more of the following:
(1) preventing a condition, disease, or disorder; for example, preventing the condition, disease, or disorder in an individual that may be predisposed to the condition, disease, or disorder but does not yet experience or display the pathology or symptomatology of the disease;
(2) inhibiting a condition, disease, or disorder; for example, inhibiting the condition, disease, or disorder in an individual that is experiencing or displaying the pathology or symptomatology of the condition, disease, or disorder [i.e., arresting (or slowing) further development of the pathology or symptomatology or both]; and
(3) ameliorating a condition, disease, or disorder; for example, ameliorating the condition, disease, or disorder in an individual that is experiencing or displaying the pathology or symptomatology of the condition, disease, or disorder [i.e., reversing the pathology or symptomatology or both].

Typically, a compound of the invention is administered in an amount effective to treat a condition, disease, or disorder as described herein. The compounds of the invention may be administered as compound in the free form, or alternatively, as a pharmaceutically acceptable salt. For administration and dosing purposes, the compound in free form or pharmaceutically acceptable salt thereof will simply be referred to as the compounds of the invention.

The compounds of the invention are administered by any suitable route in the form of a pharmaceutical composition adapted to such a route, and in a dose effective for the treatment intended. Administration of the compounds of this invention can be via any method which delivers a compound of this invention systemically and/or locally. The compounds of the invention may be administered orally, rectally, vaginally, parenterally (including, e.g., intravenous, subcutaneous, intramuscular, intravascular or infusion), topically, intranasally, or by inhalation.

The compounds of the invention may be administered orally. Oral administration may involve swallowing, so that the compound enters the gastrointestinal tract, or buccal or sublingual administration may be employed by which the compound enters the bloodstream directly from the mouth.

In another embodiment, the compounds of the invention may also be administered parenterally, for example directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors, and infusion techniques.

In another embodiment, the compounds of the invention may also be administered topically to the skin or mucosa, that is, dermally or transdermally. In another embodiment, the compounds of the invention may also be administered intranasally or by inhalation. In another embodiment, the compounds of the invention may be administered rectally or vaginally. In another embodiment, the compounds of the invention may also be administered directly to the eye or ear.

The dosage regimen for the compounds of the invention or compositions containing said compounds is based on a variety of factors, including the type, age, weight, sex and medical condition of the patient; the severity of the condition; the route of administration; and the activity of the particular compound employed. Thus, the dosage regimen may vary widely. In one embodiment, the total daily dose of a compound of the invention is typically from about 0.0001 to about 100 mg/kg (i.e., mg compound of the invention per kg body weight) for the treatment of the indicated conditions discussed herein. In another embodiment, total daily dose of the compound of the invention is from about 0.01 to about 50 mg/kg; and in another embodiment, from about 0.1 to about 50 mg/kg; and in another embodiment, from about 0.5 to about 30 mg/kg. It is not uncommon that the administration of the compounds of the invention will be repeated a plurality of times in a day (typically no greater than 4 times). Multiple doses per day typically may be used to increase the total daily dose, if desired.

### Methods and Uses

Another embodiment of the present invention includes a compound of Formula I or a pharmaceutically acceptable salt of the compound for use as a medicament, particularly wherein the medicament is for use in the treatment or prevention of a GIPR-related condition, disease, or disorder, including administering to a mammal, such as a human, in need of such treatment.

Another embodiment of the present invention includes use of a compound of Formula I or a pharmaceutically acceptable salt of the compound as a medicament, particularly wherein the medicament is for use in the treatment or prevention of a GIPR-related condition, disease, or disorder, including administering to a mammal, such as a human, in need of such treatment.

Another embodiment of the present invention includes use of a compound of Formula I or a pharmaceutically acceptable salt of the compound in the manufacture of a medicament for treating or preventing a GIPR-related condition, disease, or disorder, including administering to a mammal, such as a human, in need of such treatment a therapeutically effective amount.

Another embodiment of the present invention includes the compound of invention for use as a medicament, particularly wherein the medicament is for use in treating or preventing a condition, disease, or disorder selected from diabetes [e.g. Type 1 diabetes mellitus (T1D), Type 2 diabetes mellitus (T2DM), including pre-diabetes], idiopathic T1D (Type 1b), latent autoimmune diabetes in adults (LADA), early-onset T2DM (EOD), youth-onset atypical diabetes (YOAD), maturity onset diabetes of the young (MODY), malnutrition-related diabetes, gestational diabetes, hyperglycemia, insulin resistance, hepatic insulin resistance, impaired glucose tolerance, diabetic neuropathy, diabetic nephropathy, kidney disease [e.g., acute kidney disorder, tubular dysfunction, proinflammatory changes to the proximal tubules, or chronic kidney disease (CKD)], diabetic retinopathy, adipocyte dysfunction, visceral adipose deposition, sleep apnea [e.g. obstructive sleep apnea (OSA)], obesity (including hypothalamic obesity and monogenic obesity) and related comorbidities (e.g., osteoarthritis and urine incontinence), eating disorders (including binge eating syndrome, bulimia nervosa, and syndromic obesity such as Prader-Willi and Bardet-Biedl syndromes), weight gain such as weight gain caused by use of other agents (e.g., caused by use of steroids and/or antipsychotics, or caused by treatment of depression, or caused by use of agents on cognitive function), excessive sugar craving, dyslipidemia [including hyperlipidemia, hypertriglyceridemia, increased total cholesterol, high LDL (low-density lipoprotein) cholesterol, and low HDL (high-density lipoprotein) cholesterol], hyperinsulinemia, nonalcoholic fatty liver disease [NAFLD, including related diseases such as steatosis, nonalcoholic steatohepatitis (NASH), fibrosis, cirrhosis, and hepatocellular carcinoma], cardiovascular disease, atherosclerosis (including coronary artery disease), peripheral vascular disease, hypertension, endothelial dysfunction, impaired vascular compliance, heart failure [e.g. congestive heart failure, heart failure with preserved ejection fraction (HFpEF), heart failure with reduced ejection fraction (HFrEF)], myocardial infarction (e.g. necrosis and apoptosis), stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, osteoarthritis, Parkinson's disease, left ventricular hypertrophy, peripheral arterial disease (PAD), macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, polycystic ovary syndrome (PCOS), and addiction (e.g., addition to alcohol, nicotine, and/or drug).

Another embodiment of the present invention includes use of the compound of invention as a medicament, particularly wherein the medicament is for use in the treatment or prevention of a condition, disease, or disorder selected from diabetes [e.g. Type 1 diabetes mellitus (T1D), Type 2 diabetes mellitus (T2DM), including pre-diabetes], idiopathic T1D (Type 1b), latent autoimmune diabetes in adults (LADA), early-onset T2DM (EOD), youth-onset atypical diabetes (YOAD), maturity onset diabetes of the young (MODY), malnutrition-related diabetes, gestational diabetes, hyperglycemia, insulin resistance, hepatic insulin resistance, impaired glucose tolerance, diabetic neuropathy, diabetic nephropathy, kidney disease [e.g., acute kidney disorder, tubular dysfunction, proinflammatory changes to the proximal tubules, or chronic kidney disease (CKD)], diabetic retinopathy, adipocyte dysfunction, visceral adipose deposition, sleep apnea [e.g. obstructive sleep apnea (OSA)], obesity (including hypothalamic obesity and monogenic obesity) and related comorbidities (e.g., osteoarthritis and urine incontinence), eating disorders (including binge eating syndrome, bulimia nervosa, and syndromic obesity such as Prader-Willi and Bardet-Biedl syndromes), weight gain such as weight gain caused by use of other agents (e.g., caused by use of steroids and/or antipsychotics, or caused by treatment of depression, or caused by use of agents on cognitive function), excessive sugar craving, dyslipidemia [including hyperlipidemia, hypertriglyceridemia, increased total cholesterol, high LDL (low-density lipoprotein) cholesterol, and low HDL (high-density lipoprotein) cholesterol], hyperinsulinemia, nonalcoholic fatty liver disease [NAFLD, including related diseases such as steatosis, nonalcoholic steatohepatitis (NASH), fibrosis, cirrhosis, and hepatocellular carcinoma], cardiovascular disease, atherosclerosis (including coronary artery disease), peripheral vascular disease, hypertension, endothelial dysfunction, impaired vascular compliance, heart failure [e.g. congestive heart failure, heart failure with preserved ejection fraction (HFpEF), heart failure with reduced ejection fraction (HFrEF)], myocardial infarction (e.g. necrosis and apoptosis), stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, osteoarthritis, Parkinson's disease, left ventricular hypertrophy, peripheral arterial disease (PAD), macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, polycystic ovary syndrome (PCOS), and addiction (e.g., addition to alcohol, nicotine, and/or drug).

Another embodiment of the present invention includes use of the compound of invention for the manufacture of a medicament for treating or preventing a condition, disease, or disorder selected from diabetes [e.g. Type 1 diabetes mellitus (T1 D), Type 2 diabetes mellitus (T2DM), including pre-diabetes], idiopathic T1D (Type 1b), latent autoimmune diabetes in adults (LADA), early-onset T2DM (EOD), youth-onset atypical diabetes (YOAD), maturity onset diabetes of the young (MODY), malnutrition-related diabetes, gestational diabetes, hyperglycemia, insulin resistance, hepatic insulin resistance, impaired glucose tolerance, diabetic neuropathy, diabetic nephropathy, kidney disease [e.g., acute kidney disorder, tubular dysfunction, proinflammatory changes to the proximal tubules, or chronic kidney disease (CKD)], diabetic retinopathy, adipocyte dysfunction, visceral adipose deposition, sleep apnea [e.g. obstructive sleep apnea (OSA)], obesity (including hypothalamic obesity and monogenic obesity) and related comorbidities (e.g., osteoarthritis and urine incontinence), eating disorders (including binge eating syndrome, bulimia nervosa, and syndromic obesity such as Prader-Willi and Bardet-Biedl syndromes), weight gain such as weight gain caused by use of other agents (e.g., caused by use of steroids and/or antipsychotics, or caused by treatment of depression, or caused by use of agents on cognitive function), excessive sugar craving, dyslipidemia [including hyperlipidemia, hypertriglyceridemia, increased total cholesterol, high LDL (low-density lipoprotein) cholesterol, and low HDL (high-density lipoprotein) cholesterol], hyperinsulinemia, nonalcoholic fatty liver disease [NAFLD, including related diseases such as steatosis, nonalcoholic steatohepatitis (NASH), fibrosis, cirrhosis, and hepatocellular carcinoma], cardiovascular disease, atherosclerosis (including coronary artery disease), peripheral vascular disease, hypertension, endothelial dysfunction, impaired vascular compliance, heart failure [e.g. congestive heart failure, heart failure with preserved ejection fraction (HFpEF), heart failure with reduced ejection fraction (HFrEF)], myocardial infarction (e.g. necrosis and apoptosis), stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, osteoarthritis, Parkinson's disease, left ventricular hypertrophy, peripheral arterial disease (PAD), macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, polycystic ovary syndrome (PCOS), and addiction (e.g., addition to alcohol, nicotine, and/or drug).

In some further embodiments of the methods and uses of the present invention described herein, the condition, disease, or disorder that can be treated or prevented in accordance with the present invention is selected from obesity, T2DM, Heart Failure (e.g. HFpEF and HFrEF); CKD; NAFLD, NASH, atherosclerosis, PAD, obstructive sleep apnea, diabetic retinopathy, and diabetic neuropathy.

The compound of the invention is a GIPR antagonist. Thus, the present invention further provides a method for modulating (e.g. antagonizing) GIPR (either in vitro or in vivo), comprising contacting (including incubating) the GIPR with the compound of Formula I or a pharmaceutically acceptable salt thereof (such as one selected from Examples 1-58 herein) described herein.

In some embodiments, the amount of the compound of the invention used in any one of the methods (or uses) of the present invention is effective in antagonizing GIPR.

### Stereoisomers

The compounds of the present invention may contain asymmetric or chiral centers, and, therefore, exist in two or more stereoisomeric forms. Unless specified otherwise, it is intended that all stereoisomeric forms of the compounds of the present invention as well as mixtures thereof, including racemic mixtures, form part of the present invention. In addition, the present invention embraces all geometric and positional isomers. For example, if a compound of the present invention incorporates a double bond or a fused ring, both the cis- and trans-forms, as well as mixtures, are embraced within the scope of the invention.

Stereoisomers of the compounds may include cis and trans isomers (geometric isomers), optical isomers such as R and S enantiomers, diastereomers, rotational isomers, atropisomers, and conformational isomers. For example, compounds of the invention containing one or more asymmetric carbon atoms may exist as two or more stereoisomers. Where a compound of the invention contains an alkenyl or alkenylene group, geometric cis/trans (or Z/E) isomers are possible. Cis/trans isomers may also exist for saturated rings.

The pharmaceutically acceptable salts of compounds of the invention may also contain a counterion which is optically active (e.g., D-lactate or L-lysine) or racemic (e.g. DL-tartrate or DL-arginine).

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high-pressure liquid chromatography (HPLC). Alternatively, the racemate (or a racemic precursor) may be reacted with a suitable optically active compound, for example, an alcohol, or, in the case where a compound of the invention contains an acidic or basic moiety, a base or acid such as 1-phenylethylamine or tartaric acid. The resulting diastereomeric mixture may be separated by chromatography, fractional crystallization, or by using both of said techniques, and one or both of the diastereoisomers converted to the corresponding pure enantiomer(s) by means well known to a skilled person. Chiral compounds of the invention (and chiral precursors thereof) may be obtained in enantiomerically-enriched form using chromatography, typically HPLC. Concentration of the eluate affords the enriched mixture. Chiral chromatography using sub- and supercritical fluids may be employed. Methods for chiral chromatography useful in some embodiments of the present invention are known in the art (see, for example, Smith, Roger M., Loughborough University, Loughborough, UK; Chromatographic Science Series (1998), 75 (Supercritical Fluid Chromatography with Packed Columns), pp. 223-249 and references cited therein).

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two crystal forms are produced in equimolar amounts each comprising a single enantiomer. While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

Chiral compounds of the invention (and chiral precursors thereof) may be obtained in enantiomerically enriched form using chromatography, typically high-pressure liquid chromatography (HPLC) or supercritical fluid chromatography (SFC), on a resin with an asymmetric stationary phase and with a mobile phase consisting of a hydrocarbon, typically heptane or hexane, containing from 0 to 50% isopropanol, typically from 2 to 20%, and from 0 to 5% of an alkylamine, typically 0.1% diethylamine (DEA) or isopropylamine. Concentration of the eluent affords the enriched mixture. In the case where SFC is used, the mobile phase may consist of a supercritical fluid, typically carbon dioxide, containing 2-50% of an alcohol, such as methanol, ethanol or isopropanol.

Diastereomeric mixtures can be separated into their individual diastereoisomers on the basis of their physicochemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Enantiomers can also be separated by use of a chiral HPLC column. Alternatively, the specific stereoisomers may be synthesized by using an optically active starting material, by asymmetric synthesis using optically active reagents, substrates, catalysts or solvents, or by converting one stereoisomer into the other by asymmetric transformation.

In some embodiments, the compounds of the invention may have asymmetric carbon atoms. The carbon-carbon bonds of the compounds of Formula I may be depicted herein using a solid line ( [symbol] ), a wavy line ( [symbol] ), a solid wedge ( [symbol] ), or a dotted wedge ( [symbol] ). The use of a solid line to depict bonds to asymmetric carbon atoms is meant to indicate that all possible stereoisomers (e.g., specific enantiomers, racemic mixtures, etc.) at that carbon atom are included. The use of either a solid or dotted wedge to depict bonds to asymmetric carbon atoms is meant to indicate that only the stereoisomer shown is meant to be included. The use of a wavy line to depict bonds to asymmetric carbon atoms is meant to indicate that the stereochemistry is unknown (unless otherwise specified). It is possible that compounds of the invention may contain more than one asymmetric carbon atom. In those compounds, the use of a solid line to depict bonds to asymmetric carbon atoms is meant to indicate that all possible stereoisomers are meant to be included. For example, unless stated otherwise, it is intended that the compounds of the invention can exist as enantiomers and diastereomers or as racemates and mixtures thereof. The use of a solid line to depict bonds to one or more asymmetric carbon atoms in a compound of the invention and the use of a solid or dotted wedge to depict bonds to other asymmetric carbon atoms in the same compound is meant to indicate that a mixture of diastereomers is present.

Where the compounds of the present invention possess two or more stereogenic centers and the absolute or relative stereochemistry is given in the name, the designations R and S refer respectively to each stereogenic center in ascending numerical order (1, 2, 3, etc.) according to the conventional IUPAC number schemes for each molecule. Where the compounds of the present invention possess one or more stereogenic centers and no stereochemistry is given in the name or structure, it is understood that the name or structure is intended to encompass all forms of the compound, including the racemic form.

The compounds of this invention may contain olefin-like double bonds or ring structures. When such bonds or ring structures are present, the compounds of the invention can exist as cis and/or trans configurations and as mixtures thereof. For example, when a double bond is present, when the two higher-priority groups (at each side of the double bond) are oriented in the same direction, the stereoisomer is referred to as cis, whereas when the two higher-priority groups are oriented in opposing directions, the stereoisomer is referred to as trans. The term “cis” can also refer to the orientation of two substituents with reference to each other and the plane of the ring (either both “up” or both “down”). Analogously, the term “trans” can also refer to the orientation of two substituents with reference to each other and the plane of the ring (the substituents being on opposite sides of the ring).

Included within the scope of the claimed compounds of the present invention are all stereoisomers, geometric isomers and tautomeric forms of the compounds of the invention, including compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

### Tautomerism

Where structural isomers are interconvertible via a low-energy barrier, tautomeric isomerism (‘tautomerism’) may occur. This may take the form of proton tautomerism in compounds of the invention containing, for example, an imino/amino, keto/enol, or oxime/nitroso group, lactam/lactim or so-called valence tautomerism in compounds which contain an aromatic moiety. It follows that a single compound may exhibit more than one type of isomerism.

It must be emphasized that while, for conciseness, the compounds of the invention have been drawn herein in a single tautomeric form, all possible tautomeric forms are included within the scope of the invention.

It is possible that the intermediates and compounds of the present invention may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations.

Valence tautomers include interconversions by reorganization of some of the bonding electrons.

### Isotopes

The present invention includes all pharmaceutically acceptable isotopically labelled compounds of the invention wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I, ¹²⁴I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S

Certain isotopically labelled compounds of Formula I, for example, those incorporating a radioactive isotope, are useful in drug and/or substrate tissue distribution studies. The radioactive isotopes tritium, i.e., ³H, and carbon-14, i.e., ¹⁴C, are particularly useful for this purpose in view of their ease of incorporation and ready means of detection.

Substitution with heavier isotopes such as deuterium, i.e., ²H, may afford certain therapeutic advantages resulting from greater metabolic stability, for example, increased in vivo half-life or reduced dosage requirements, and hence may be preferred in some circumstances.

In some embodiments, the disclosure provides deuterium-labeled (or deuterated) compounds and salts, where the formula and variables of such compounds and salts are each and independently as described herein. “Deuterated” means that at least one of the atoms in the compound is deuterium in an abundance that is greater than the natural abundance of deuterium (typically approximately 0.015%). A skilled artisan recognized that in chemical compounds with a hydrogen atom, the hydrogen atom actually represents a mixture of H and D, with about 0.015% being D. The concentration of the deuterium incorporated into the deuterium-labeled compounds and salt of the invention may be defined by the deuterium enrichment factor. It is understood that one or more deuterium may exchange with hydrogen under physiological conditions.

In some embodiments, one or more hydrogen atoms on certain metabolic sites on the compounds of the invention may be deuterated. MetaSite (moldiscovery.com/software/metasite/) may be helpful in predicting some metabolic sites on the compounds of the invention.

Substitution with positron-emitting isotopes, such as ¹¹C, ¹⁸F, ¹⁵O and ¹³N, can be useful in Positron Emission Tomography (PET) studies for examining substrate receptor occupancy.

Isotopically labelled compounds of the invention can generally be prepared by conventional techniques known to those skilled in the art or by processes analogous to those described in the accompanying Examples and Preparations using an appropriate isotopically labelled reagent in place of the non-labelled reagent previously employed.

Pharmaceutically acceptable solvates (including hydrates) in accordance with the invention include those wherein the solvent of crystallization may be isotopically substituted, e.g., D₂O, d6-acetone, d6-DMSO.

### Salts

The compounds of the present invention may be isolated and used per se, or when possible, in the form of its pharmaceutically acceptable salt. The term “salts” refers to inorganic and organic salts of a compound of the present invention. These salts can be prepared in situ during the final isolation and purification of a compound, or by separately treating the compound with a suitable organic or inorganic acid or base and isolating the salt thus formed.

Salts encompassed within the term “pharmaceutically acceptable salts” refer to the compounds of the invention which are generally prepared by reacting the free base with a suitable organic or inorganic acid to provide a salt of the compound of the invention that is suitable for administration to a patient, or by reacting the free acid with a suitable organic or inorganic base to provide a salt of the compound of the invention that is suitable for administration to a patient.

In addition, the compounds of the invention may also include other salts of such compounds which are not necessarily pharmaceutically acceptable salts, which may be useful as intermediates for one or more of the following: 1) preparing compounds of Formula I; 2) purifying compounds of Formula I; 3) separating enantiomers of compounds of Formula I; or 4) separating diastereomers of compounds of Formula I.

Suitable base salts are formed from bases which form non-toxic salts. Examples include, but are not limited to aluminum, ammonium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulfate and hemicalcium salts.

For a review on suitable salts, see Paulekun, G. S. et al., Trends in Active Pharmaceutical Ingredient Salt Selection Based on Analysis of the Orange Book Database, J. Med. Chem. 2007; 50(26), 6665-6672.

Pharmaceutically acceptable salts of compounds of the invention may be prepared by methods well known to one skilled in the art, including but not limited to the following procedures
(i) by reacting a compound of the invention with the desired acid or base;
(ii) by removing an acid- or base-labile protecting group from a suitable precursor of a compound of the invention or by ring-opening a suitable cyclic precursor, for example, a lactone or lactam, using the desired acid or base; or
(iii) by converting one salt of a compound of the invention to another. This may be accomplished by reaction with an appropriate acid or base or by means of a suitable ion exchange procedure.

These procedures are typically carried out in solution. The resulting salt may precipitate out and be collected by filtration or may be recovered by evaporation of the solvent.

### Solvates

The compounds of the invention (e.g. a compound of Formula I or pharmaceutically acceptable salts thereof) may exist in unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

In addition, the compounds of the invention may also include other solvates of such compounds that are not necessarily pharmaceutically acceptable solvates, which may be useful as intermediates for one or more of the following: 1) preparing compounds of Formula I or their salts; 2) purifying compounds of Formula I or their salts; 3) separating enantiomers of compounds of Formula I or their salts; or 4) separating diastereomers of compounds of Formula I or their salts.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm.

### Complexes

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallization, by recrystallization from solvents, or by physically grinding the components together—see O. Almarsson and M. J. Zaworotko, Chem. Commun., 17, 1889-1896 (2004). For a general review of multi-component complexes, see Haleblian, J. Pharm. Sci., 64 (8), 1269-1288 (1975).

### Prodrugs

Also included within the scope of the invention are prodrugs of the compounds of the invention. A compound of the invention may be administered in the form of a prodrug. Thus, certain derivatives of a compound of the invention which may have little or no pharmacological activity themselves may, when administered into or onto the body, be converted into a compound of the invention having the desired activity, for example by hydrolytic cleavage, particularly hydrolytic cleavage promoted by an esterase or peptidase enzyme. Such derivatives are referred to as ‘prodrugs’. Further information on the use of prodrugs may be found in ‘The Expanding Role of Prodrugs in Contemporary Drug Design and Development, Nature Reviews Drug Discovery, 17, 559-587 (2018) (J. Rautio et al.).

Prodrugs in accordance with the invention may, for example, be produced by replacing appropriate functionalities present in compounds of the invention with certain moieties known to those skilled in the art as ‘pro-moieties’ as described, for example, in ‘Design of Prodrugs’ by H. Bundgaard (Elsevier, 1985).

Thus, a prodrug in accordance with the invention may be (a) an ester or amide derivative of a carboxylic acid when present in a compound of the invention; (b) an ester, carbonate, carbamate, phosphate or ether derivative of a hydroxyl group when present in a compound of the invention; (c) an amide, imine, carbamate or amine derivative of an amino group when present in a compound of the invention; (d) a thioester, thiocarbonate, thiocarbamate or sulfide derivatives of a thiol group when present in a compound of the invention; or (e) an oxime or imine derivative of a carbonyl group when present in a compound of the invention.

Some specific examples of prodrugs in accordance with the invention include:
(i) when a compound of the invention contains a carboxylic acid functionality (—COOH), an ester thereof, such as a compound wherein the hydrogen of the carboxylic acid functionality of the compound is replaced by C₁-C₃ alkyl (e.g., ethyl) or (C₁-C₃ alkyl)C(═O)OCH₂— (e.g., ^{t}BuC(═O)OCH₂—);
(ii) when a compound of the invention contains an alcohol functionality (—OH), an ester thereof, such as a compound wherein the hydrogen of the alcohol functionality of the compound is replaced by —CO(C₁-C₈ alkyl) (e.g., methylcarbonyl) or the alcohol is esterified with an amino acid;
(iii) when a compound of the invention contains an alcohol functionality (—OH), an ether thereof, such as a compound wherein the hydrogen of the alcohol functionality of the compound is replaced by (C₁-C₈ alkyl)C(═O)OCH₂—or —CH₂OP(═O)(OH)₂;
(iv) when a compound of the invention contains an alcohol functionality (—OH), a phosphate thereof, such as a compound wherein the hydrogen of the alcohol functionality of the compound is replaced by —P(═O)(OH)₂ or —P(═O)(O⁻Na⁺)₂ or —P(═O)(O⁻)₂Ca²⁺;
(v) when a compound of the invention contains a primary or secondary amino functionality (—NH₂ or —NHR where R≠H), an amide thereof, for example, a compound wherein, as the case may be, one or both hydrogens of the amino functionality of the compound is/are replaced by (C₁-C₁₀)alkanoyl, —COCH₂NH₂ or the amino group is derivatized with an amino acid; (vi) when a compound of the invention contains a primary or secondary amino functionality (—NH₂ or —NHR where R≠H), an amine thereof, for example, a compound wherein, as the case may be, one or both hydrogens of the amino functionality of the compound is/are replaced by —CH₂OP(═O)(OH)₂.

Certain compounds of the invention may themselves act as prodrugs of other compounds the invention. It is also possible for two compounds of the invention to be joined together in the form of a prodrug. In certain circumstances, a prodrug of a compound of the invention may be created by internally linking two functional groups in a compound of the invention, for instance by forming a lactone.

### Metabolites

Also included within the scope of the invention are active metabolites of compounds of Formula I (including prodrugs) or their pharmaceutically acceptable salts, that is, compounds formed in vivo upon administration of the drug, often by oxidation or dealkylation. Some examples of metabolites in accordance with the invention include:
(i) where the compound of Formula I or its pharmaceutically acceptable salt contains a methyl group, a hydroxymethyl derivative thereof (—CH₃—>—CH₂OH) and
(ii) where the compound of Formula I or its pharmaceutically acceptable salt contains an alkoxy group, a hydroxy derivative thereof (—OR—>—OH).

Also included within the scope of the invention are active metabolites of compounds of the invention, that is, compounds formed in vivo upon administration of the drug, often by oxidation or dealkylation. Some examples of metabolites in accordance with the invention include, but are not limited to,
(i) where the compound of the invention contains an alkyl group, a hydroxyalkyl derivative thereof (—CH—>—COH):
(ii) where the compound of the invention contains an alkoxy group, a hydroxy derivative thereof (—OR—>—OH);
(iii) where the compound of the invention contains a tertiary amino group, a secondary amino derivative thereof (—NRR′—>—NHR or —NHR′);
(iv) where the compound of the invention contains a secondary amino group, a primary derivative thereof (—NHR—>—NH₂);
(v) where the compound of the invention contains a phenyl moiety, a phenol derivative thereof (-Ph->-PhOH);
(vi) where the compound of the invention contains an amide group, a carboxylic acid derivative thereof (—CONH₂—>COOH); and
(vii) where the compound contains a hydroxy or carboxylic acid group, the compound may be metabolized by conjugation, for example with glucuronic acid to form a glucuronide. Other routes of conjugative metabolism exist. These pathways are frequently known as Phase 2 metabolism and include, for example, sulfation or acetylation. Other functional groups, such as NH groups, may also be subject to conjugation.

### Solid form

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid.

Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

The compounds of the invention may also exist in a mesomorphic state (mesophase or liquid crystal) when subjected to suitable conditions. The mesomorphic state is intermediate between the true crystalline state and the true liquid state (either melt or solution) and consists of two dimensional order on the molecular level. Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K+, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

Certain compounds of the present invention may exist in more than one crystal form (generally referred to as “polymorphs”). Polymorphs may be prepared by crystallization under various conditions, for example, using different solvents or different solvent mixtures for recrystallization; crystallization at different temperatures; and/or various modes of cooling, ranging from very fast to very slow cooling during crystallization. Polymorphs may also be obtained by heating or melting the compound of the present invention followed by gradual or fast cooling. The presence of polymorphs may be determined by solid probe NMR spectroscopy, IR spectroscopy, differential scanning calorimetry, powder X-ray diffraction or such other techniques.

In general the compounds of this invention can be made by processes which include processes analogous to those known in the chemical arts, particularly in light of the description contained herein. Certain processes for the manufacture of the compounds of this invention are provided as further features of the invention and are illustrated by the following reaction schemes. Other processes may be described in the experimental section. Specific synthetic schemes for preparation of the compounds of Formula I or their pharmaceutically acceptable salts are outlined below. Note that tetrazoles are generally a high-energy functional group and care should be taken in the synthesis and handling of tetrazole-containing molecules.

### Co-Administration

The compounds of the invention may be used alone, or in combination with one or more other therapeutic agents. The invention provides any of the uses, methods or compositions as defined herein wherein the compound of the invention, or pharmaceutically acceptable salt thereof, is used in combination with one or more other therapeutic agent discussed herein.

The administration of two or more compounds “in combination” means that all of the compounds are administered closely enough in time to affect treatment of the subject. The two or more compounds may be administered simultaneously or sequentially, via the same or different routes of administration, on same or different administration schedules and with or without specific time limits depending on the treatment regimen. Additionally, simultaneous administration may be carried out by mixing the compounds prior to administration or by administering the compounds at the same point in time but as separate dosage forms at the same or different site of administration.

Examples of “in combination” include, but are not limited to, “concurrent administration,” “co-administration,” “simultaneous administration,” “sequential administration” and “administered simultaneously”.

A compound of the invention and the one or more other therapeutic agents may be administered as a fixed or non-fixed combination of the active ingredients. The term “fixed combination” means a compound of the invention, or a pharmaceutically acceptable salt thereof, and the one or more therapeutic agents, are both administered to a subject simultaneously in a single composition or dosage. The term “non-fixed combination” means that a compound of the invention, or a pharmaceutically acceptable salt thereof, and the one or more therapeutic agents are formulated as separate compositions or dosages such that they may be administered to a subject in need thereof simultaneously or at different times with variable intervening time limits, wherein such administration provides effective levels of the two or more compounds in the body of the subject.

The combination agents are administered to a patient (e.g. a mammal or human) in a therapeutically effective amount. By “therapeutically effective amount” it is meant an amount of a compound of the present invention that, when administered alone or in combination with an additional therapeutic agent to a mammal, is effective to treat the desired disease/disorder/condition (e.g., T2DM or obesity).

In some embodiments, a compound of this invention may be co-administered with one or more other agents such as Orlistat, TZDs and other insulin-sensitizing agents, FGF21 analogs, Metformin, Omega-3-acid ethyl esters (e.g., Lovaza), Fibrates, HMG CoA-reductase Inhibitors, Ezetimibe, Probucol, Ursodeoxycholic acid, TGR5 agonists, FXR agonists, Vitamin E, Betaine, Pentoxifylline, CB1 antagonists, Carnitine, N-acetylcysteine, Reduced glutathione, lorcaserin, the combination of naltrexone with buproprion, SGLT2 inhibitors (including dapagliflozin, canagliflozin, empagliflozin, tofogliflozin, ertugliflozin, ASP-1941, THR1474, TS-071, ISIS388626 and LX4211 as well as those in WO2010023594), Phentermine, Topiramate, GLP-1 receptor agonists, GIP receptor agonists, GIP receptor inhibitors and/or antagonists, dual GLP-1 receptor/glucagon receptor agonists (e.g., OPK88003, MED10382, JNJ-64565111, NN9277, BI 456906), dual GLP-1 receptor/GIP receptor agonists [e.g., Tirzepatide (LY3298176), NN9423, NN9541, HS-20094, SCO-094, VK2735, CT-388, GMA-106, CT-868, HRS9531], dual GLP-1 receptor/glucagon receptor agonists (e.g. DD-01, PB-718, mazdutide, pemvidutide, pegapamodutide, survodutide, LM-008, IBI-362, AZD9550), dual GLP-1 receptor/GLP-2 receptor agonists (e.g. dapiglutide), dual GLP-1 receptor/amylin receptor agonists (e.g. amycretin), cagrilinitide/semaglutide, GLP-1 receptor agonist/GIP receptor antagonist (maridebart cafraglutide), dual GLP-1 receptor/FGF21 receptor agonists (e.g. HEC-88473, BI 3006337), triple agonists of the GLP-1 receptor/glucagon receptor/GIP receptor (e.g. retatrutide), triple agonists of the GLP-1 receptor/glucagon receptor/FGF21 receptor (e.g. DR10624), NPY2 receptor agonists (e.g. BI 1820237), activin receptor type-2B modulators (e.g. bimagrumab), amylin receptor agonists, GPR75 modulators, delta-5 desaturase inhibitors, orexin 2 receptor modulators, Angiotensin-receptor blockers, an acetyl-CoA carboxylase (ACC) inhibitor, a ketohexokinase (KHK) inhibitor, ASK1 inhibitors, branched-chain alpha-keto acid dehydrogenase kinase inhibitors (BCKDK inhibitors), inhibitors of CCR2 and/or CCR5, PNPLA3 inhibitors, DGAT1 inhibitors, DGAT2 inhibitors, an FGF21 analog, FGF19 analogs, PPAR agonists, FXR agonists, AMPK activators [e.g., ETC-1002 (bempedoic acid)], SCD1 inhibitors or MPO inhibitors.

Exemplary GLP-1 receptor agonists include liraglutide, albiglutide, exenatide, lixisenatide, dulaglutide, semaglutide, danuglipron, orforglipron, lotiglipron, PF-06954522, HM15211, LY3298176, Medi-0382, NN-9924, TTP-054, TTP-273, efpeglenatide, CT-996, ECC5004, XW004, XW014, MDR-001, ZT002, KN-056, GL0034, GSBR-1290, noiiglutide, RGT-075, TTP-273, HRS-7535, GMA-105, TG103, GZR-18, GX-G6, ecnoglutide, PB-119, QLG2065, beinaglutide, those described in WO2018109607, those described in WO2019239319 (PCT/IB2019/054867 filed Jun. 11, 2019), and those described in WO2019239371 (PCT/IB2019/054961 filed Jun. 13, 2019).

Exemplary ACC inhibitors include 4-(4-[(1-isopropyl-7-oxo-1,4,6,7-tetrahydro-1′H-spiro[indazole-5,4′-piperidin]-1′-yl)carbonyl]-6-methoxypyridin-2-yl)benzoic acid, gemcabene, and firsocostat (GS-0976) and pharmaceutically acceptable salts thereof.

Exemplary FXR agonists include tropifexor (2-[(1R,3R,5S)-3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylic acid), cilofexor (GS-9674), obeticholic acid, LY2562175, Met409, TERN-101 and EDP-305 and pharmaceutically acceptable salts thereof.

Exemplary KHK inhibitors include [(1R,5S,6R)-3-{2-[(2S)-2-methylazetidin-1-yl]-6-(trifluoromethyl)pyrimidin-4-yl}-3-azabicyclo[3.1.0]hex-6-yl]acetic acid and pharmaceutically acceptable salts thereof.

Exemplary DGAT2 inhibitors include (S)-2-(5-((3-ethoxypyridin-2-yl)oxy)pyridin-3-yl)—N-(tetrahydrofuran-3-yl)pyrimidine-5-carboxamide [including its crystalline solid forms (Form 1 and Form 2)]. See U.S. Pat. No. 10,071,992.

Some exemplary BCKDK inhibitors include those described in U.S. Pat. Nos. 11,542,270 and 11,059,833, including the following:
5-(5-chloro-4-fluoro 3-methylthiophen-2-yl)-1H-tetrazole;
5-(5-chloro-3-difluoromethylthiophen-2-yl)-1H-tetrazole;
5-(5-fluoro-3-methylthiophen-2-yl)-1H-tetrazole;
5-(5-chloro-3-methylthiophen-2-yl)-1H-tetrazole;
5-(3,5-dichlorothiophen-2-yl)-1H-tetrazole;
5-(4-bromo-3-methylthiophen-2-yl)-1H-tetrazole;
5-(4-bromo-3-ethylthiophen-2-yl)-1H-tetrazole;
5-(4-chloro-3-ethylthiophen-2-yl)-1H-tetrazole;
3-chloro-5-fluorothieno[3,2-b]thiophene-2-carboxylic acid;
3-bromo-5-fluorothieno[3,2-b]thiophene-2-carboxylic acid;
3-(difluoromethyl)-5-fluorothieno[3,2-b]thiophene-2-carboxylic acid;
5,6-difluorothieno[3,2-b]thiophene-2-carboxylic acid; and
3,5-difluorothieno[3,2-b]thiophene-2-carboxylic acid;
   or a pharmaceutically acceptable salt thereof.

Some additional exemplary BCKDK inhibitors include those described in U.S. patent application Ser. No. 18/060,027, filed Nov. 30, 2022, including the following:
6-fluoro-3-(2,4,6-trifluoro-3-methoxyphenyl)-1-benzothiophene-2-carboxylic acid;
6-fluoro-3-(2,4,5-trifluoro-3-methoxyphenyl)-1-benzothiophene-2-carboxylic acid;
6-chloro-3-(2,4,5-trifluoro-3-methylphenyl)-1-benzothiophene-2-carboxylic acid;
6-chloro-3-(2,4-difluoro-3-methoxyphenyl)-1-benzothiophene-2-carboxylic acid;
3-(6-chloro-2,4-difluoro-3-methoxyphenyl)-6-fluoro-1-benzothiophene-2-carboxylic acid;
3-(6-chloro-2,4-difluoro-3-methoxyphenyl)-6-fluoro-1-benzothiophene-2-carboxylic acid, ATROP-2;
3-(3-chloro-2,4,5-trifluorophenyl)-6-fluoro-1-benzothiophene-2-carboxylic acid;
3-(4-chloro-2,6-difluoro-3-methoxyphenyl)-6-fluoro-1-benzothiophene-2-carboxylic acid;
6-chloro-3-(2,4,6-trifluoro-3-methoxyphenyl)-1-benzothiophene-2-carboxylic acid;
6-chloro-3-(3-ethyl-2,4,5-trifluorophenyl)-1-benzothiophene-2-carboxylic acid; or
ammonium 3-(3-ethyl-2,4,5-trifluorophenyl)-6-fluoro-1-benzothiophene-2-carboxylate;
   or a pharmaceutically acceptable salt thereof.

In some embodiments, a compound of this invention may be co-administered with one or more anti-diabetic agents. Suitable anti-diabetic agents include insulin, metformin, GLP-1 receptor agonists (described herein above), an acetyl-CoA carboxylase (ACC) inhibitor (described herein above), SGLT2 inhibitors (described herein above), monoacylglycerol O-acyltransferase inhibitors, phosphodiesterase (PDE)-10 inhibitors, AMPK activators [e.g., ETC-1002 (bempedoic acid)], sulfonylureas (e.g., acetohexamide, chlorpropamide, diabinese, glibenclamide, glipizide, glyburide, glimepiride, gliclazide, glipentide, gliquidone, glisolamide, tolazamide, and tolbutamide), meglitinides, α-amylase inhibitors (e.g., tendamistat, trestatin and AL-3688), an α-glucoside hydrolase inhibitor (e.g., acarbose), α-glucosidase inhibitors (e.g., adiposine, camiglibose, emiglitate, miglitol, voglibose, pradimicin-Q, and salbostatin), PPARγ agonists (e.g., balaglitazone, ciglitazone, darglitazone, englitazone, isaglitazone, pioglitazone and rosiglitazone), PPAR α/γ agonists (e.g., CLX-0940, GW-1536, GW-1929, GW-2433, KRP-297, L-796449, LR-90, MK-0767 and SB-219994), protein tyrosine phosphatase-1B (PTP-1B) inhibitors [e.g., trodusquemine, hyrtiosal extract, and compounds disclosed by Zhang, S. et al., Drug Discovery Today, 12(9/10), 373-381 (2007)], SIRT-1 activators (e.g., resveratrol, GSK2245840 or GSK184072), dipeptidyl peptidase IV (DPP-IV) inhibitors (e.g., those in WO2005116014, sitagliptin, vildagliptin, alogliptin, dutogliptin, linagliptin and saxagliptin), insulin secretagogues, fatty acid oxidation inhibitors, A2 antagonists, c-jun amino-terminal kinase (JNK) inhibitors, glucokinase activators (GKa) such as those described in WO2010103437, WO2010103438, WO2010013161, WO2007122482, TTP-399, TTP-355, TTP-547, AZD1656, ARRY403, MK-0599, TAK-329, AZD5658 or GKM-001, insulin, insulin mimetics, glycogen phosphorylase inhibitors (e.g., GSK1362885), VPAC2 receptor agonists, glucagon receptor modulators such as those described in Demong, D. E. et al., Annual Reports in Medicinal Chemistry 2008, 43, 119-137, GPR119 modulators, particularly agonists, such as those described in WO2010140092, WO2010128425, WO2010128414, WO2010106457, Jones, R. M. et al., Annual Reports in Medicinal Chemistry 2009, 44, 149-170 (e.g., MBX-2982, GSK1292263, APD597 and PSN821), FGF21 derivatives or analogs such as those described in Kharitonenkov, A. et al., Current Opinion in Investigational Drugs 2009, 10(4)₃₅₉-364, TGR5 (also termed GPBAR1) receptor modulators, particularly agonists, such as those described in Zhong, M., Current Topics in Medicinal Chemistry, 2010, 10(4), 386-396 and INT777, GPR40 agonists, such as those described in Medina, J. C., Annual Reports in Medicinal Chemistry, 2008, 43, 75-85, including but not limited to TAK-875, GPR120 modulators, particularly agonists, high-affinity nicotinic acid receptor (HM74A) activators, and SGLT1 inhibitors, such as GSK1614235. A further representative listing of anti-diabetic agents that can be combined with the compounds of the present invention can be found, for example, at page 28, line 35 through page 30, line 19 of WO2011005611.

Other antidiabetic agents could include inhibitors or modulators of carnitine palmitoyl transferase enzymes, inhibitors of fructose 1,6-diphosphatase, inhibitors of aldose reductase, mineralocorticoid receptor inhibitors, inhibitors of TORC2, inhibitors of CCR2 and/or CCR5, inhibitors of PKC isoforms (e.g., PKCα, PKCβ, PKCγ), inhibitors of fatty acid synthetase, inhibitors of serine palmitoyl transferase, modulators of GPR81, GPR39, GPR43, GPR41, GPR105, Kv1.3, retinol binding protein 4, glucocorticoid receptor, somatostain receptors (e.g., SSTR1, SSTR2, SSTR3 and SSTR5), inhibitors or modulators of PDHK2 or PDHK4, inhibitors of MAP4K4, modulators of IL1 family including IL1beta, and modulators of RXRalpha. In addition suitable anti-diabetic agents include mechanisms listed by Carpino, P. A., Goodwin, B. Expert Opin. Ther. Pat., 2010, 20(12), 1627-51.

The compounds of the present invention may be co-administered with anti-heart failure agents such as ACE inhibitors (e.g., captopril, enalapril, fosinopril, lisinopril, perindopril, quinapril, ramipril, trandolapril), Angiotensin II receptor blockers (e.g., candesartan, losartan, valsartan), Angiotensin-receptor neprilysin inhibitors (sacubitril/valsartan), I_{f} channel blocker Ivabradine, Beta-Adrenergic blocking agents (e.g., bisoprolol, metoprolol succinate, carvedilol), Aldosterone antagonists (e.g., spironolactone, eplerenone), hydralazine and isosorbide dinitrate, diuretics (e.g., furosemide, bumetanide, torsemide, chlorothiazide, amiloride, hydrochlorothiazide, Indapamide, Metolazone, Triamterene), or digoxin.

The compounds of the present invention may also be co-administered with cholesterol or lipid lowering agents including the following exemplary agents: HMG CoA reductase inhibitors (e.g., pravastatin, pitavastatin, lovastatin, atorvastatin, simvastatin, fluvastatin, NK-104 (a.k.a. itavastatin, or nisvastatin or nisbastatin) and ZD-4522 (a.k.a. rosuvastatin, or atavastatin or visastatin); squalene synthetase inhibitors; fibrates (e.g., gemfibrozil, pemafibrate, fenofibrate, clofibrate); bile acid sequestrants (such as questran, colestipol, colesevelam); ACAT inhibitors; MTP inhibitors; lipooxygenase inhibitors; cholesterol absorption inhibitors (e.g., ezetimibe); nicotinic acid agents (e.g., niacin, niacor, slo-niacin); omega-3 fatty acids (e.g., epanova, fish oil, eicosapentaenoic acid); cholesteryl ester transfer protein inhibitors (e.g., obicetrapib) and PCSK9 modulators [e.g., alirocumab, evolocumab, bococizumab, ALN-PCS (inclisiran)].

The compounds of the present invention may also be used in combination with antihypertensive agents and such antihypertensive activity is readily determined by those skilled in the art according to standard assays (e.g., blood pressure measurements). Examples of suitable anti-hypertensive agents include: alpha-adrenergic blockers; beta-adrenergic blockers; calcium channel blockers (e.g., diltiazem, verapamil, nifedipine and amlodipine); vasodilators (e.g., hydralazine), diruetics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, torsemide, furosemide, musolimine, bumetanide, triamtrenene, amiloride, spironolactone); renin inhibitors; ACE inhibitors (e.g., captopril, zofenopril, fosinopril, enalapril, ceranopril, cilazopril, delapril, pentopril, quinapril, ramipril, lisinopril); AT-1 receptor antagonists (e.g., losartan, irbesartan, valsartan); ET receptor antagonists (e.g., sitaxsentan, atrsentan and compounds disclosed in U.S. Pat. Nos. 5,612,359 and 6,043,265); Dual ET/All antagonist (e.g., compounds disclosed in WO 00/01389); neutral endopeptidase (NEP) inhibitors; vasopepsidase inhibitors (dual NEP-ACE inhibitors) (e.g., gemopatrilat and nitrates). An exemplary antianginal agent is ivabradine.

Examples of suitable calcium channel blockers (L-type or T-type) include diltiazem, verapamil, nifedipine and amlodipine and mybefradil.

Examples of suitable cardiac glycosides include digitalis and ouabain.

In one embodiment, a compound of invention may be co-administered with one or more diuretics. Examples of suitable diuretics include (a) loop diuretics such as furosemide (such as LASIX™), torsemide (such as DEMADEX™), bemetanide (such as BUMEX™), and ethacrynic acid (such as EDECRIN™); (b) thiazide-type diuretics such as chlorothiazide (such as DIURIL™ ESIDRIX™ or HYDRODIURIL™), hydrochlorothiazide (such as MICROZIDE™ or ORETIC™) benzthiazide, hydroflumethiazide (such as SALURON™), bendroflumethiazide, methychlorthiazide, polythiazide, trichlormethiazide, and indapamide (such as LOZOL™); (c) phthalimidine-type diuretics such as chlorthalidone (such as HYGROTON™), and metolazone (such as ZAROXOLYN™); (d) quinazoline-type diuretics such as quinethazone; and (e) potassium-sparing diuretics such as triamterene (such as DYRENIUM™), and amiloride (such as MIDAMOR™ or MODURETIC™) In another embodiment, a compound of the invention may be co-administered with a loop diuretic. In still another embodiment, the loop diuretic is selected from furosemide and torsemide.

In still another embodiment, one or more compounds of Formula I or their pharmaceutically acceptable salts may be co-administered with furosemide. In still another embodiment, one or more compounds of Formula I or their pharmaceutically acceptable salts may be co-administered with torsemide which may optionally be a controlled or modified release form of torsemide.

In another embodiment, a compound of the invention may be co-administered with a thiazide-type diuretic. In still another embodiment, the thiazide-type diuretic is selected from the group consisting of chlorothiazide and hydrochlorothiazide. In still another embodiment, one or more compounds of Formula I or their pharmaceutically acceptable salts may be co-administered with chlorothiazide. In still another embodiment, one or more compounds of Formula I or their pharmaceutically acceptable salts may be co-administered with hydrochlorothiazide.

In another embodiment, one or more compounds of Formula I or their pharmaceutically acceptable salts may be co-administered with a phthalimidine-type diuretic. In still another embodiment, the phthalimidine-type diuretic is chlorthalidone.

Examples of suitable mineralocorticoid receptor antagonists include sprionolactone and eplerenone.

Examples of suitable phosphodiesterase inhibitors include: PDE Ill inhibitors (such as cilostazol); and PDE V inhibitors (such as sildenafil).

Those skilled in the art will recognize that the compounds of this invention may also be used in conjunction with other cardiovascular or cerebrovascular treatments including Percutaneous Coronary Intervention (PCI), stenting, drug-eluting stents, stem cell therapy and medical devices such as implanted pacemakers, defibrillators, or cardiac resynchronization therapy.

Particularly when provided as a single dosage unit, the potential exists for a chemical interaction between the combined active ingredients. For this reason, when a compound of this invention and a second therapeutic agent are combined in a single dosage unit they may be formulated such that although the active ingredients are combined in a single dosage unit, the physical contact between the active ingredients is minimized (that is, reduced). For example, one active ingredient may be enteric-coated. By enteric-coating one of the active ingredients, it is possible not only to minimize the contact between the combined active ingredients, but also, it is possible to control the release of one of these components in the gastrointestinal tract such that one of these components is not released in the stomach but rather is released in the intestines. One of the active ingredients may also be coated with a material that effects a sustained release throughout the gastrointestinal tract and also serves to minimize physical contact between the combined active ingredients. Furthermore, the sustained-released component can be additionally enteric-coated such that the release of this component occurs only in the intestine. Still another approach would involve the formulation of a combination product in which the one component is coated with a sustained and/or enteric-release polymer, and the other component is also coated with a polymer such as a low viscosity grade of hydroxypropyl methylcellulose (HPMC) or other appropriate materials as known in the art, in order to further separate the active components. The polymer coating serves to form an additional barrier to interaction with the other component.

These as well as other ways of minimizing contact between the components of combination products of the present invention, whether administered in a single dosage form or administered in separate forms but at the same time by the same manner, will be readily apparent to those skilled in the art, once armed with the present disclosure.

Another approach may involve the formulation of a combination product in which both active components are combined with a material that effects a sustained release throughout the gastrointestinal tract of both active ingredients.

In some embodiments of combination therapy treatment, both the compounds of this invention and the other drug therapies are administered to patients such as mammals (e.g., humans, male or female) by conventional methods.

### Kits

Another aspect of the invention provides kits comprising the compound of the invention or pharmaceutical compositions comprising the compound of the invention. A kit may include, in addition to the compound of the invention or pharmaceutical composition thereof, diagnostic or therapeutic agents. A kit may also include instructions for use in a diagnostic or therapeutic method. In some embodiments, the kit includes the compound or a pharmaceutical composition thereof and a diagnostic agent. In other embodiments, the kit includes the compound or a pharmaceutical composition thereof and one or more therapeutic agents as described in the co-administration section hereinabove.

In yet another embodiment, the invention comprises kits that are suitable for use in performing the methods of treatment described herein. In one embodiment, the kit contains a first dosage form comprising one or more of the compounds of the invention in quantities sufficient to carry out the methods of the invention. In another embodiment, the kit comprises one or more compounds of the invention in quantities sufficient to carry out the methods of the invention and a container for the dosage and a container for the dosage.

### Synthesis

Compounds of the present invention may be synthesized by synthetic routes that include processes analogous to those well-known in the chemical arts, particularly in light of the description contained herein. The starting materials are generally available from commercial sources or may be prepared using methods well known to those skilled in the art. Many of the compounds used herein are related to, or may be derived from, compounds in which one or more of the scientific interest or commercial need has occurred. Accordingly, such compounds may be one or more of 1) commercially available; 2) reported in the literature or 3) prepared from other commonly available substances by one skilled in the art using materials which have been reported in the literature.

For illustrative purposes, the reaction schemes depicted below provide potential routes for synthesizing the compounds of the present invention as well as key intermediates. For a more detailed description of the individual reaction steps, see the Examples section below. Those skilled in the art will appreciate that other synthetic routes may be used to synthesize the inventive compounds. Although specific starting materials and reagents are discussed below, other starting materials and reagents may be substituted to provide one or more of a variety of derivatives or reaction conditions. In addition, many of the compounds prepared by the methods described below may be further modified in light of this disclosure using conventional chemistry well known to those skilled in the art.

The skilled person will appreciate that the experimental conditions set forth in the schemes that follow are illustrative of suitable conditions for effecting the transformations shown, and that it may be necessary or desirable to vary the precise conditions employed for the preparation of compounds of the invention. It will be further appreciated that it may be necessary or desirable to carry out the transformations in a different order from that described in the schemes, or to modify one or more of the transformations, to provide the desired compound of the invention.

In the preparation of compounds of the invention it is noted that some of the preparation methods useful for the preparation of the compounds described herein may require protection of remote functionality (e.g., a primary amine, secondary amine, carboxyl, etc. in a precursor of a compound of the invention). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see March's Advanced Organic Chemistry: Reactions, Mechanisms, and Structure 8th Edition.

For example, if a compound contains a amine or carboxylic acid functionality, such functionality may interfere with reactions at other sites of the molecule if left unprotected. Accordingly, such functionalities may be protected by an appropriate protecting group (PG) which may be removed in a subsequent step. Suitable protecting groups for amine and carboxylic acid protection include those protecting groups commonly used in peptide synthesis (such as N-t-butoxycarbonyl (Boc), benzyloxycarbonyl (Cbz), and 9-fluorenylmethylenoxycarbonyl (Fmoc) for amines and lower alkyl or benzyl esters for carboxylic acids) which are generally not chemically reactive under the reaction conditions described and may typically be removed without chemically altering other functionality in a compound of the invention.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high-performance liquid chromatography (HPLC) or thin-layer chromatography (TLC).

Compounds of Formula I, salts and intermediates thereof may be prepared according to the following reaction schemes and accompanying discussion. The reaction schemes described below are intended to provide a general description of the methodology employed in the preparation of the compounds of the present invention. Some of the compounds of the present invention contain a single chiral center with stereochemical designation (R or S) and others will contain two separate chiral centers with stereochemical designation (R or S). It will be apparent to one skilled in the art that most of the synthetic transformations can be conducted in a similar manner whether the materials are enantioenriched or racemic. Moreover, the resolution to the desired optically active material may take place at any desired point in the sequence using well known methods such as described herein and in the chemistry literature.

Unless otherwise indicated, in the reaction schemes that follow, variables R¹, R², R³, R^{cy1}, R^{L1}R^{L2}R^{A}, A¹, L¹, L², L^{Q1}, L^{Q2}, T¹, T², T³, T⁴, T⁵, T⁶, T⁷, T⁸, n1, t1, t2, and t3 and structural Formula I (including, e.g., Formula Ia) in the reaction schemes and discussion that follow are as defined herein or consistent with those described in the claims and embodiments herein. For each of the variables (including those are not in the claims or embodiments, e.g. R or R′), its meaning remains the same as initially described unless otherwise indicated in a later occurrence. In general, the compounds of this invention may be made by processes which include processes analogous to those known in the chemical arts, particularly in light of the description contained herein. Certain processes for the manufacture of the compounds of this invention and intermediates thereof are provided as further features of the invention and are illustrated by the following reaction schemes. Other processes are described in the experimental section. The schemes and examples provided herein (including the corresponding description) are for illustration only, and not intended to limit the scope of the present invention.

In general, the compounds of this invention may be made by processes described herein and by analogous processes known to those skilled in the art. Certain processes for the manufacture of the compounds of this invention are described in the following reaction schemes. Other processes are described in the experimental section. The schemes and examples provided herein (including the corresponding description) are for illustration only. One skilled in the art will recognize that intermediates and compounds of Formula I prepared according to the following schemes may be isolated as salts or non-salts depending on the conditions of the reaction, isolation, or purification. One skilled in the art will also recognize that in some instances, additional synthetic steps may be required to protect and deprotect certain functional groups present within the synthetic sequence. One skilled in the art will further recognize that in other instances, certain functional groups may be carried through the synthetic sequences described and then may be transformed into alternate substituents present in compounds of Formula 1.

Scheme 1 refers to the preparation of compounds of Formula II (Compounds of Formula I wherein L^{Q1} is C(═O) and L^{QZ} is NH) wherein L² is NH from amino acids of structure 1-1.

Compounds of structure 1-1 can be reacted with isocyanates of structure 1-2 in the presence of a base (such as N,N-diisopropylethylamine) in a suitable solvent (such as tetrahydrofuran) to afford ureas of structure 1-4. Alternatively, ureas of structure 1-4 can be obtained by reacting amines of structure 1-3 with a suitable reactant (such as triphosgene or 1,1′-carbonyldiimidazole) to form an intermediate and subsequently reacting the resulting intermediate with a compound of structure 1-1. One skilled in the art will recognize that numerous alternate conditions may be selected for the formation of ureas (such as ureas of structure 1-4). (See e.g. J. Med. Chem. 2020, 63, 2751-2788). Compounds of Formula I can be prepared from an amide bond forming reaction between carboxylic acid intermediate 1-4 and amine intermediate 1-5. Amide bond forming reactions of this type can be achieved by combining a carboxylic acid (such as carboxylic acid structure 1-4) with an amine (such as amine of structure 1-5) in the presence of an activating reagent (such as 1-ethyl-3-(3-dimethylaminopropyl)-carbodiimide, 2,4,6-tripropyl-1,3,5,2,4,6-trioxatriphosphorinane-2,4,6-trioxide, or 1-hydroxybenzotriazole) and a base (such as 1-methyl imidazole or N,N-diisopropylethylamine) in a suitable solvent (such as dichloromethane). One skilled in the art will recognize that numerous alternate conditions may be selected for the formation of an amide (such as a compound of Formula I) from a carboxylic acid (such as a carboxylic acid of structure 1-4) and an amine (such as an amine of structure 1-5). (See e.g. Chem. Rev. 2011, 111, 6557-6602). If R³ contains an ester protecting group, it can be deprotected using appropriate conditions such as trifluoroacetic acid (if ester is t-butyl) to afford compounds with a carboxylic acid in R³, which are also examples of compounds of Formula 1.

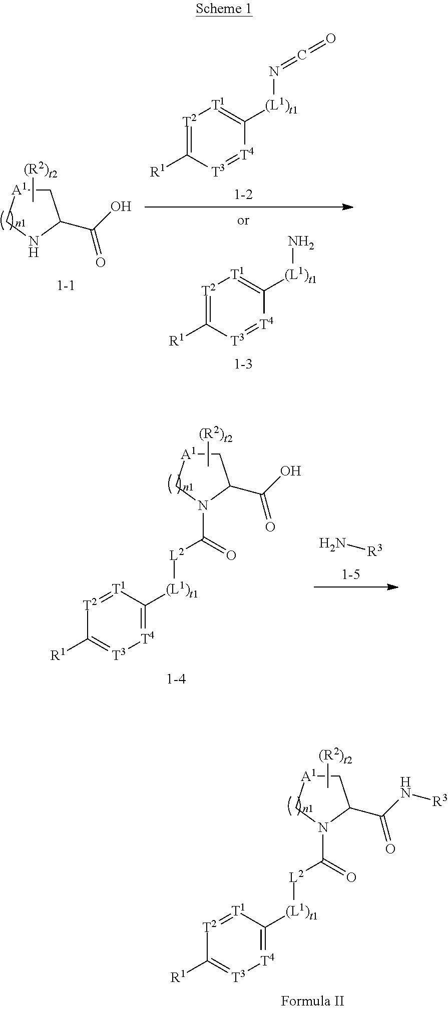

For example, as shown in Scheme 1a, an amine 1-5a (wherein PG¹ is an acid protecting group, for example an alkyl group—i.e., the acid moiety in the compound 1-5a is protected as its corresponding ester) can be reacted with an acid 1-4 to afford compound 1-6a (wherein its acid moiety is protected), which subsequently can be deprotected to form the corresponding acid 1-6b under appropriate conditions.

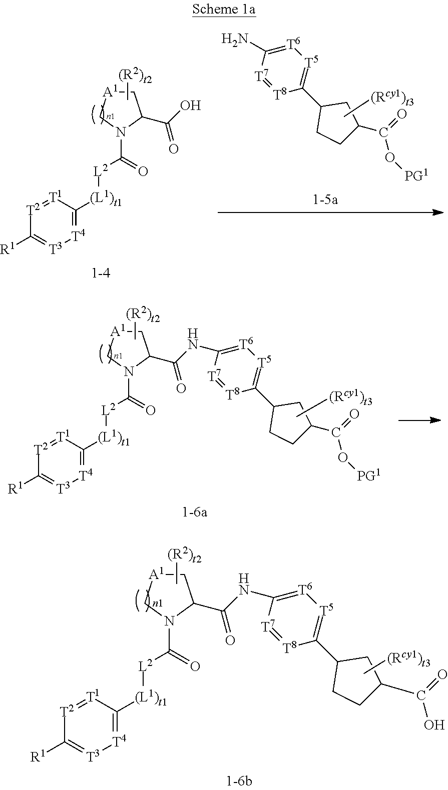

Scheme 2 refers to the preparation of compounds of Formula II wherein L² is NH from nitrogen-protected amino acids of structure 2-1. A carboxylic acid of structure 2-1 can be reacted with an amine of structure 1-5 via amide bond forming conditions as described in Scheme 1. The remaining tert-butyloxycarbonyl protecting group in the resulting amide can be removed upon treatment with an acid such as trifluoroacetic acid to afford the amine intermediate of structure 2-2. The intermediate of structure 2-2 can be coupled with an isocyanate of structure 1-2 or an amine of structure 1-3 via urea-forming conditions as described previously in Scheme 1 to afford compounds of Formula I. One skilled in the art will recognize that an alternative protecting group from the Boc shown in structure 2-1 may also be used. For example, fluorenylmethyloxycarbonyl (Fmoc), another protecting group, could be used in place of the Boc group of structure 2-1. After amidation, the Fmoc can be subsequently removed by conditions known to one skilled in the art, such as stirring with piperidine in a solvent such as N,N-dimethylformamide. Another protecting group, benzyloxycarbonyl, could also be used in place of the Boc group of structure 2-1. If R³ contains an ester protecting group, it can be deprotected using appropriate conditions such as trifluoroacetic acid (if ester is t-butyl) to afford compounds with a carboxylic acid in R³, which are also examples of compounds of Formula II.

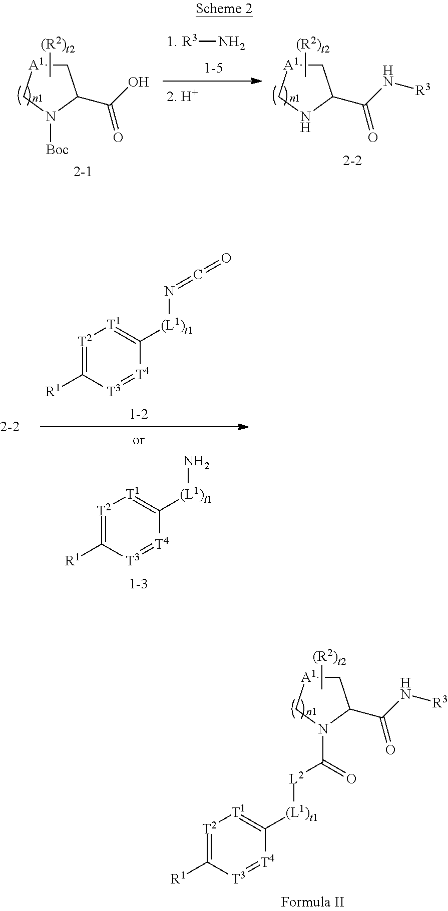

Scheme 3 refers to the preparation of compounds of structure 3-2 which are a type of structure of Formula II in which L² is C(R^{L2})₂ from amino amides of structure 2-2 that can be made according to the methods of Scheme 2. A carboxylic acid of structure 3-1 can be reacted with an amine of structure 2-2 via amide bond forming conditions as described in Scheme 1. Amide bond forming reactions of this type can be achieved by combining a carboxylic acid (such as carboxylic acid structure 3-1) with an amine (such as amine of structure 2-2) in the presence of an activating reagent (such as 1-ethyl-3-(3-dimethylaminopropyl)-carbodiimide, 2,4,6-tripropyl-1,3,5,2,4,6-trioxatriphosphorinane-2,4,6-trioxide, or 1-hydroxybenzotriazole) and a base (such as 1-methyl imidazole or N,N-diisopropylethylamine) in a suitable solvent (such as dichloromethane). One skilled in the art will recognize that numerous alternate conditions may be selected for the formation of an amide (such as a compound of structure 3-2) from a carboxylic acid (such as a carboxylic acid of structure 3-1) and an amine (such as an amine of structure 2-2). (See e.g. Chem. Rev. 2011, 111, 6557-6602). If R³ contains an ester protecting group, it can be deprotected using appropriate conditions such as trifluoroacetic acid (if ester is t-butyl) to afford compounds with a carboxylic acid in R³, which are also examples of structure 3-2, which are also compounds of Formula I.

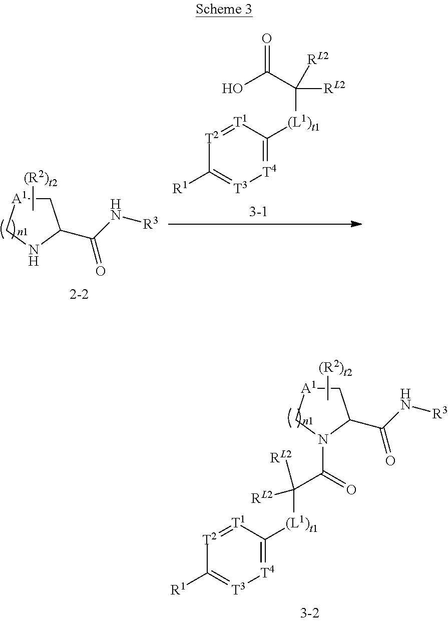

Scheme 4 refers to the preparation of compounds of structure 3-2, which are compounds of Formula II in which L² is C(R^{L2})₂. Carboxylic acids of structure 3-1 can be coupled with compounds of structure 4-1 to form amide-esters which can subsequently be hydrolyzed to amide-acids of structure 4-2 using acidic or basic hydrolysis conditions. Coupling between acid 3-1 and amino esters of structure 4-1 is well exemplified in the literature and there are many conditions known to one skilled in the art that can be used to effect this transformation such as amide bond forming conditions detailed in Scheme 1. The conversion of ester to acid 4-2 is also well known to those skilled in the art and could be acid or base mediated. One set of suitable conditions include the use of lithium hydroxide to enact this hydrolysis transformation. Acid 4-2 can be further elaborated with a subsequent amide bond forming reaction as described in Scheme 1 to afford compounds of structure 3-2, which are examples of compounds of Formula II.

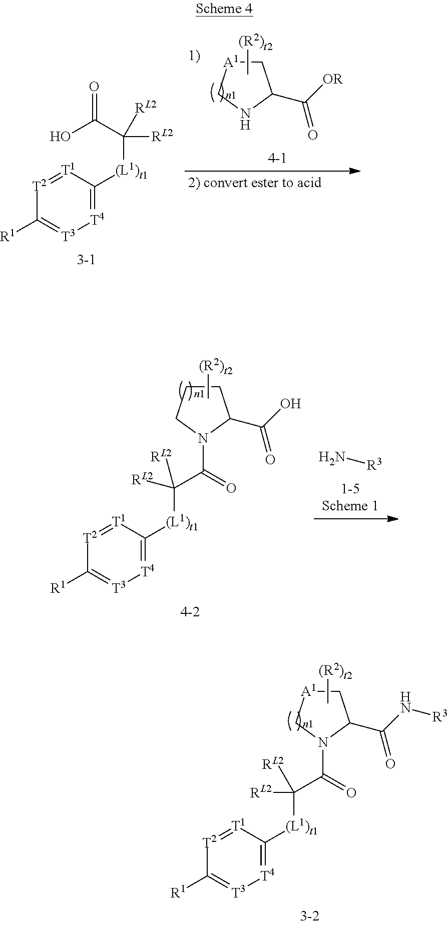

Scheme 5 refers to the preparation of compounds of structure 5-5 which are a type of compound of Formula I (wherein L^{Q1} is CH₂ and L^{Q2} is O). Alcohols of structure 5-1 can be converted into a leaving group under conditions such as 4-methylbenzene-1-sulfonyl chloride in pyridine. In a second step the resulting tosylate can be reacted with alcohols of structure 5-2 to arrive at ethers of structure 5-3. Conditions to effect this transformation could include a base such as cesium carbonate in a suitable solvent such as acetonitrile. One skilled in the art would recognize that other leaving groups could be substituted for the tosylate shown in Scheme 10. The Boc group of 5-3 can be removed under acidic conditions as detailed above to arrive at amines of structure 5-4. Amines of structure 5-4 can be converted to structures 5-5 wherein L² is NH by reaction with either structures 1-2 or 1-3 under the urea forming conditions as described above.

One skilled in the art will recognize that protecting groups other than Boc could also be used in structure 5-1. Structures 5-5 are a type of compound of Formula I. If R³ contains an ester function group, which are a type of compound of Formula I, it can subsequently be hydrolyzed to a carboxylic acid which is another R³ containing group that is a type of compound of Formula I.

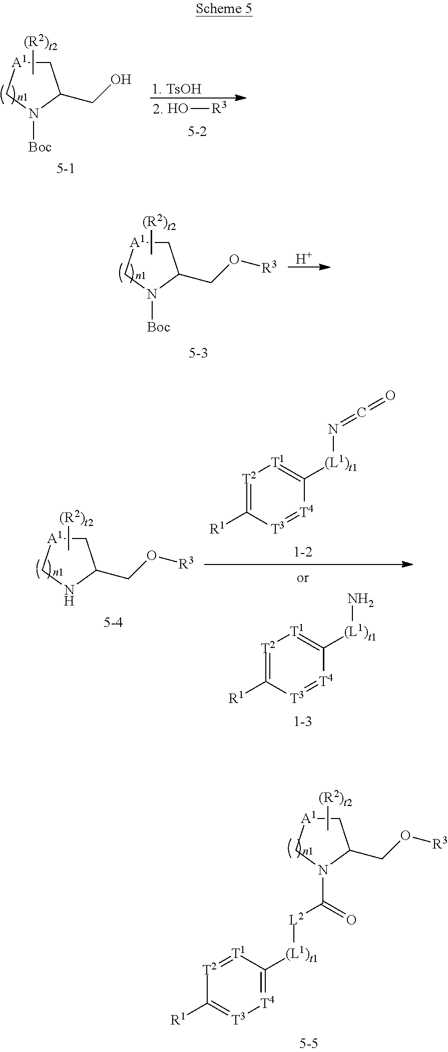

Scheme 6 depicts a method of making an aniline derivative 6-3 (which is an example of compound of structure 1-3). An intermediate bromide 6-1 can be coupled with a compound of structure 6-2 to afford the intermediate 6-3. An aryl bromide or heteroaryl of structure 6-1 can be reacted with, for example, a vinyl boronic ester 6-2 (wherein R¹ is, for example, vinyl, alkenyl, or the like; each R″ is H, alkyl, or the like, or two OR″, together with the boron atom to which they are attached, optionally form a 4-7-membered heterocylcoalkyl optionally substituted with one or more substituents such as alkyl, or the like) in the presence of an appropriate catalyst (such as [1,1′-bis(diphenylphosphino)ferrocene]dichloropalladium(II)), a base (such as potassium carbonate) in a solvent (such as 1,4-dioxane) to afford 6-3. If the intermediate of structure 6-3 contains a functional group, additional transformation can be effected to yield another compound of structure 6-3. For example, if a compound of structure 6-3 contains an alkenyl group, hydrogenation of that the alkenyl group using a catalyst (such as palladium on carbon) will provide another compound of structure 6-3 containing a resultant alkyl group (which is also an example of a compound of structure 1-2).

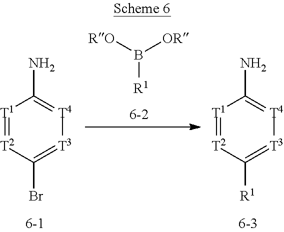

Scheme 7 refers to the preparation of compounds of structure 7-5 from boronic acid or ester derivatives 7-2 (wherein each of R″ is independently H or C₁₋₄ alkyl; or two OR″, together with the boron atom to which they are attached, form a heterocycloalkyl that is optionally substituted with one more C₁₋₄ alkyl). Compounds of structures 7-2 can be coupled with aryl halides of structure 7-1 (wherein X is halogen, for example Br), via a Suzuki reaction (e.g. Acc Chem Res. 2008, 1461-1473) to afford intermediates 7-3. A Suzuki reaction is the coupling of an aryl or vinyl boronic acid with an aryl or vinyl halide using a palladium catalyst. For example, an vinyl boronate of structure 7-2 can be reacted with an aryl halide of structure 7-1 in the presence of a base (such as sodium carbonate) and a palladium catalyst (such as bis(triphenylphosphine)palladium(II) dichloride) in a suitable solvent (such as 1,4-dioxane) to afford the intermediate olefin 7-3. Reduction of the olefin can be accomplished under a number of conditions known to those skilled in the art to provide compounds of structure 7-4. For example, the reduction may be effected by exposure to palladium on carbon under an atmosphere of hydrogen gas in a suitable solvent (such as methanol). Deprotection of the amine in 7-4 can be accomplished using an acid (such as hydrochloric acid) in a suitable solvent (such as 1,4 dioxane) to afford amines of structure 7-5.

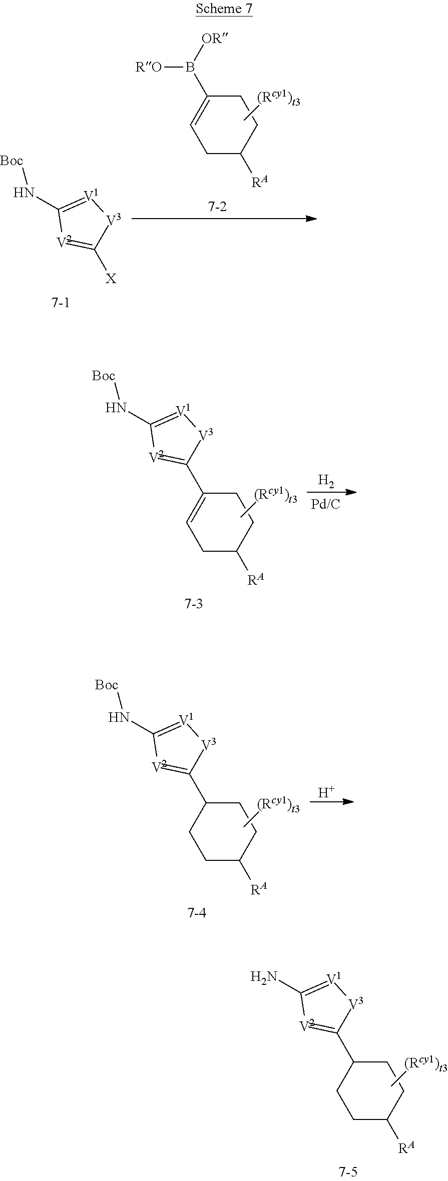

Scheme 8 refers to the preparation of compounds of structure 8-4. Compounds of structures 8-1 (wherein X is halogen, for example F) can be coupled with amines of structure 8-2, via a nucleophilic aromatic substitution reaction to afford intermediates 8-3. This reaction can be accomplished in the presence of base (such as diisopropylethylamine) and a suitable solvent. Reduction of the nitro group of structure 8-3 can be accomplished using conditions known to those skilled in the art, such as by exposure to palladium on carbon under an atmosphere of hydrogen gas in a suitable solvent to provide compounds of structure 8-4.

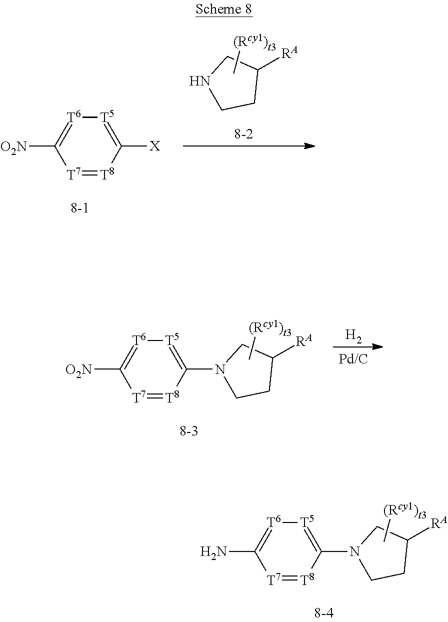

Scheme 9 refers to the preparation of compounds of structure 9-4. An alcohol of structure 9-1 can be used in a deoxygenative coupling with a halide of structure 9-2 (where X is a halogen such as CI). using metal catalyzed conditions to form sp²-sp³ bonds that are familiar to those skilled in the art. For example, alcohol 9-1 can be combined with 5,7-di-tert-butyl-3-phenylbenzo[d]oxazol-3-ium tetrafluoroborate in a suitable solvent such as methyl tert-butyl ether and pyridine to a form a reactive intermediate that can be combined with a halide such as structure 9-2, a catalyst system such as [4,4′-bis(1,1-dimethylethyl)-2,2′-bipyridine-N1,N1′]bis[2-(2-pyridinyl-N)phenyl-C]iridium(III) hexafluorophosphate, nickel(II) bromide 1,2-dimethoxyethane complex, 4,4′-dimethoxy-2,2′-bipyridine, 1-azabicyclo[2.2.2]octane, and 1H-isoindole-1,3(2H)-dione, in a suitable solvent such as N,N-dimethylacetamide. The reaction can be irradiated with blue LED light (450 nm) to provide compounds of structure 9-3. The Boc group in structure 9-3 can be removed with an acid (such as hydrochloric acid) in a suitable solvent (such as 1,4-dioxane) to provide structure 9-4.

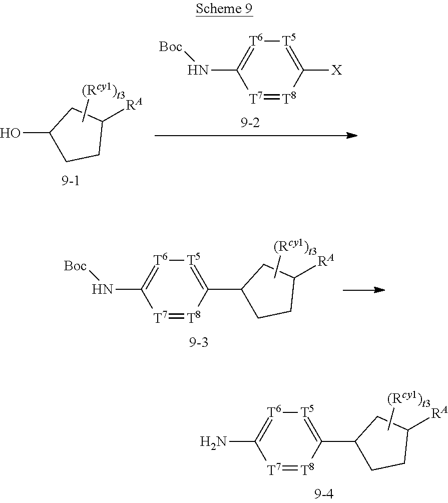

Scheme 10 refers to a preparation of chiral compounds of Formula Ia. Chiral compounds (such as compounds of structure 1-1a or 2-1a or 3-1a or 5-1a) can be transformed according to the methods described herein such as those in Schemes 1-5 to afford compounds of Formula Ia. One skilled in the art will recognize that the enantiomeric purity observed for the compounds of Formula Ia can be influenced by numerous factors (such as the synthetic sequence utilized, the reagents selected for each transformation, and the purification methods employed).

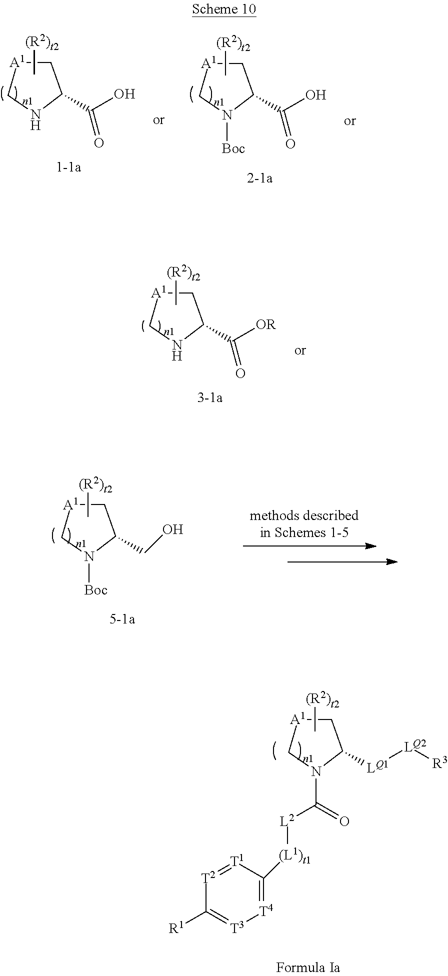

Additional starting materials and intermediates useful for making the compounds of the present invention can be obtained from chemical vendors such as Sigma-Aldrich or can be made according to methods described in the chemical art.

Those skilled in the art can recognize that in all of the Schemes described herein, if there are functional (reactive) groups present on a part of the compound structure such as a substituent group, for example R, R′, R¹, R², R³, R^{cy1}, R^{A}, A¹, L¹, L², L^{Q1}, L^{Q2}, T¹, T², T³, T⁴, T⁵, T⁶, T⁷, T⁸, etc., further modification can be made if appropriate and/or desired, using methods well known to those skilled in the art. For example, a —CN group can be hydrolyzed to afford an amide group; a carboxylic acid can be converted to an amide; a carboxylic acid can be converted to an ester, which in turn can be reduced to an alcohol, which in turn can be further modified. For another example, an OH group can be converted into a better leaving group such as a methanesulfonate, which in turn is suitable for nucleophilic substitution, such as by a cyanide ion (CN⁻). For another example, an ester group can be hydrolyzed to a carboxylic acid group. For yet another example, an unsaturated bond such as C═C or C≡C can be reduced to a saturated bond by hydrogenation. One skilled in the art will recognize further such modifications. Thus, a compound of Formula I having a substituent that contains a functional group can be converted to another compound of Formula I having a different substituent group.

Similarly, those skilled in the art can also recognize that in all of the schemes described herein, if there are functional (reactive) groups present on a substituent group such as R³ these functional groups can be protected/deprotected in the course of the synthetic scheme described here, if appropriate and/or desired. For example, an OH group can be protected by a benzyl, methyl, or acetyl group, which can be deprotected and converted back to the OH group in a later stage of the synthetic process. For another example, a carboxylic group can be protected by an alkyl group (thus forming an ester group); conversion back to the carboxylic group group can be carried out at a later stage of the synthetic process via deprotection.

As used herein, the term “reacting” (or “reaction” or “reacted”) refers to the bringing together of designated chemical reactants such that a chemical transformation takes place generating a compound different from any initially introduced into the system. Reactions can take place in the presence or absence of solvent.

A detailed description of the individual reaction steps is provided in the Example section below. Those skilled in the art will appreciate that other synthetic routes may be used to synthesize the compounds. Although specific starting materials and reagents are discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

The following illustrate the synthesis of various compounds of the present invention. Additional compounds within the scope of this invention may be prepared using the methods illustrated in these Examples, either alone or in combination with techniques generally known in the art. All starting materials in these Preparations and Examples are either commercially available or can be prepared by methods known in the art or as described herein.

Reactions were performed in air or, when oxygen- or moisture-sensitive reagents or intermediates were employed, under an inert atmosphere (nitrogen or argon). When appropriate, reaction apparatuses were dried under dynamic vacuum using a heat gun, and anhydrous solvents (Sure-Seal™ products from Sigma-Aldrich or DriSolv™ products from EMD Chemicals, Gibbstown, NJ) were employed. In some cases, commercial solvents were passed through columns packed with 4 Å molecular sieves, until the following QC standards for water were attained: a) <100 ppm for dichloromethane, toluene, N,N-dimethylformamide, and tetrahydrofuran; b) <180 ppm for methanol, ethanol, 1,4-dioxane, and diisopropylamine. For very sensitive reactions, solvents were further treated with metallic sodium, calcium hydride, or molecular sieves, and distilled just prior to use.

Other commercial solvents and reagents were used without further purification. For syntheses referencing procedures in other Examples or Methods, reaction conditions (reaction time and temperature) may vary. Products were generally dried under vacuum before being carried on to further reactions or submitted for biological testing.

When indicated, reactions were heated by microwave irradiation using Biotage Initiator or Personal Chemistry Emrys Optimizer microwave instruments. Reaction progress was monitored using thin-layer chromatography (TLC), liquid chromatography-mass spectrometry (LCMS), high-performance liquid chromatography (HPLC), and/or gas chromatography-mass spectrometry (GCMS) analyses. TLC was performed on pre-coated silica gel plates with a fluorescence indicator (254 nm excitation wavelength) and visualized under UV light and/or with I₂, KMnO₄, CoCl₂, phosphomolybdic acid, or ceric ammonium molybdate stains. LCMS data were acquired on an Agilent 1100 Series instrument with a Leap Technologies autosampler, Gemini C18 columns, acetonitrile/water gradients, and either trifluoroacetic acid, formic acid, or ammonium hydroxide modifiers. The column eluent was analyzed using a Waters ZQ mass spectrometer scanning in both positive and negative ion modes from 100 to 1200 Da. Other similar instruments were also used. HPLC data were generally acquired on an Agilent 1100 Series instrument using Gemini or XBridge C18 columns, acetonitrile/water gradients, and either trifluoroacetic acid or ammonium hydroxide modifiers. High resolution mass spectra (HRMS) were obtained on an Agilent™ Model 6210 using time of flight method. GCMS data were acquired using a Hewlett Packard 6890 oven with an HP 6890 injector, HP-1 column (12 m×0.2 mm×0.33 μm), and helium carrier gas.

Samples were analyzed on an HP 5973 mass selective detector, scanning from 50 to 550 Da using electron ionization. Purifications were generally performed by medium performance liquid chromatography (MPLC) using Isco CombiFlash Companion, AnaLogix IntelliFlash 280, Biotage SP1, or Biotage Isolera One instruments and pre-packed Isco RediSep or Biotage Snap silica cartridges. Chiral purifications were generally performed by chiral supercritical fluid chromatography (SFC) using Berger or Thar instruments; ChiralPAK-AD, -AS, —IC, Chiralcel-OD, or -OJ columns; and CO₂ mixtures with methanol, ethanol, propan-2-ol, or acetonitrile, alone or modified using trifluoroacetic acid or propan-2-amine. UV detection was used to trigger fraction collection. For syntheses referencing procedures in other Examples or Methods, purifications may vary; in general, solvents and the solvent ratios used for eluents/gradients were chosen to provide appropriate R_{f}s or retention times.

Mass spectrometry data are reported from LCMS analyses. Mass spectrometry (MS) was performed via atmospheric pressure chemical ionization (APCI), electrospray ionization (ESI), electron impact ionization (EI) or electron scatter (ES) ionization sources. Proton nuclear magnetic spectroscopy (¹H NMR) chemical shifts are given in parts per million downfield from tetramethylsilane and were recorded on 300, 400, 500, or 600 MHz Varian, Bruker, or Jeol spectrometers. Chemical shifts are expressed in parts per million (ppm, δ) referenced to the deuterated solvent residual peaks (chloroform, 7.26 ppm; CD₂HOD, 3.31 ppm; acetonitrile-d₂, 1.94 ppm; dimethyl sulfoxide-d₅, 2.50 ppm; DHO, 4.79 ppm). The peak shapes are described as follows: s, singlet; d, doublet; t, triplet; q, quartet; quin, quintet; m, multiplet; br s, broad singlet; app, apparent. Analytical SFC data were acquired on a Berger analytical instrument as described above. Optical rotation data were acquired on a PerkinElmer model 343 polarimeter using a 1 dm cell. Silica gel chromatography was performed primarily using medium-pressure Biotage or ISCO systems using columns pre-packaged by various commercial vendors including Biotage and ISCO. Microanalyses were performed by Quantitative Technologies Inc. and were within 0.4% of the calculated values.

Unless otherwise noted, chemical reactions were performed at room temperature (about 23 degrees Celsius).

Unless noted otherwise, all reactants were obtained commercially without further purifications or were prepared using methods known in the literature.

The terms “concentrated,” “evaporated,” and “concentrated in vacuo” refer to the removal of solvent at reduced pressure on a rotary evaporator with a bath temperature less than 60° C. The abbreviation “min” and “h” stand for “minutes” and “hours” respectively. The term “TLC” refers to thin-layer chromatography, “room temperature or ambient temperature” means a temperature between 18 and 25° C., “GCMS” refers to gas chromatography-mass spectrometry, “LCMS” refers to liquid chromatography-mass spectrometry, “UPLC” refers to ultra-performance liquid chromatography and “HPLC” refers to high-performance liquid chromatography, “SFC” refers to supercritical fluid chromatography.

Hydrogenation may be performed in a Parr Shaker under pressurized hydrogen gas, or in a Thales-nano H-Cube flow hydrogenation apparatus at full hydrogen and a flow rate between 1 and 2 mL/minute at the specified temperature.

HPLC, UPLC, LCMS, GCMS, and SFC retention times were measured using the methods noted in the procedures.

In some examples, chiral separations were carried out to separate enantiomers or diastereomers of certain compounds of the invention (in some examples, the separated enantiomers are designated as ENANT-1 and ENANT-2, according to their order of elution; similarly, separated diastereomers are designated as DIAST-1 and DIAST-2, according to their order of elution). In some examples, the optical rotation of an enantiomer was measured using a polarimeter. According to its observed rotation data (or its specific rotation data), an enantiomer with a clockwise rotation was designated as the (+)-enantiomer and an enantiomer with a counter-clockwise rotation was designated as the (−)-enantiomer. Racemic compounds, as well as isolated stereocenters that are an equal mixture of both (R) and (S), are indicated by the absence of drawn or described stereochemistry. Racemic compounds may also be indicated by the presence of (+/−) adjacent to the structure; in this latter case, the indicated stereochemistry represents just one of the two enantiomers that make up the racemic mixture. In the case of separated diastereomers and products derived from them, however, the presence of “DIAST-1,” “DIAST-2,” “DIAST-3,” or “DIAST-4” in the compound descriptor indicates that only one of the potential diastereomers is present in the chemical matter, although the absolute stereochemistry of the stereocenters is not yet known.

The compounds and intermediates described below were named using the naming convention provided with ACD/ChemSketch 2023.2.0, File Version C45H41, Build 137017 (Advanced Chemistry Development, Inc., Toronto, Ontario, Canada). The naming convention provided with ACD/ChemSketch 2023.2.0 is well known by those skilled in the art and it is believed that the naming convention provided with ACD/ChemSketch 2023.2.0 generally comports with the IUPAC (International Union for Pure and Applied Chemistry) recommendations on Nomenclature of Organic Chemistry and the CAS Index rules.

### PREPARATIONS

Preparations P1 to P8 describe preparations of some starting materials or intermediates used for preparation of certain compounds of the invention.

### Preparation P1

### 1-{[4-(Trifluoromethyl)phenyl]carbamoyl}-D-proline (P1)

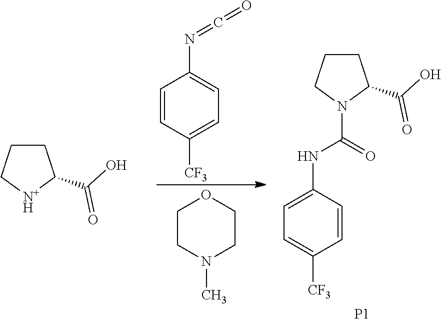

4-Methylmorpholine (3.53 mL, 32.1 mmol) and 1-isocyanato-4-(trifluoromethyl)benzene (5.00 g, 26.7 mmol) were added to a solution of D-proline (3.69 g, 32.1 mmol) in tetrahydrofuran (89 mL); after the reaction mixture had been stirred at 20° C. for 2 hours, water (100 mL) was added, followed by solid sodium bicarbonate. The resulting mixture, pH 7 to 8, was washed with methyl tert-butyl ether (2×100 mL). The aqueous layer was then acidified to pH 3 by addition of concentrated hydrochloric acid, resulting in the formation of a white precipitate. This material was collected via filtration and lyophilized for 16 hours, providing P1 as a white solid. Yield: 5.12 g, 16.9 mmol, 63%. LCMS m/z 303.0 [M+H]⁺. ¹H NMR (400 MHz, DMSO-d₆) δ 12.49 (br s, 1H), 8.69 (br s, 1H), 7.74 (d, J=8.5 Hz, 2H), 7.58 (d, J=8.6 Hz, 2H), 4.41-4.27 (m, 1H), 3.64-3.45 (m, 2H), 2.26-2.11 (m, 1H), 2.00-1.83 (m, 3H).

### Preparation P2

### 1-{[4-(Trifluoromethoxy)phenyl]carbamoyl}-D-proline (P2)

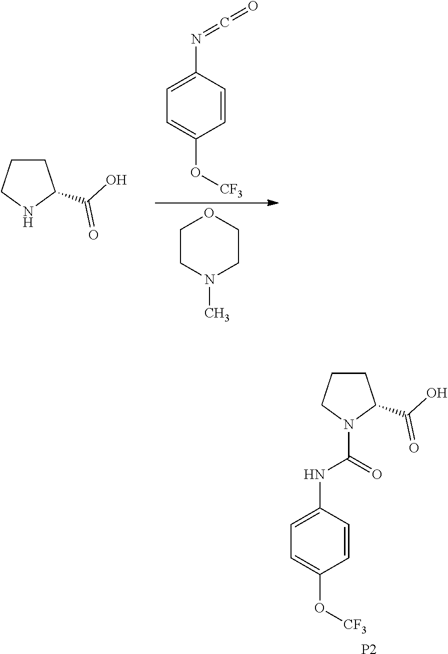

4-Methylmorpholine (5.11 mL, 46.5 mmol) and 1-isocyanato-4-(trifluoromethoxy)benzene (7.87 g, 38.7 mmol) were added to a solution of D-proline (5.35 g, 46.5 mmol) in tetrahydrofuran (129 mL), whereupon the reaction mixture was stirred at 25° C. overnight. Water (150 mL) was added, followed by solid sodium bicarbonate, which brought the mixture to a pH of 7 to 8. After the mixture had been washed with methyl tert-butyl ether (2×80 mL), the aqueous layer was acidified to pH 3 by addition of 1 M hydrochloric acid. The resulting precipitate was collected via filtration and washed with water (2×8 mL); lyophilization for 16 hours provided P2 as a white solid. Yield: 8.65 g, 27.2 mmol, 70%. LCMS m/z 319.0 [M+H]⁺. ¹H NMR (400 MHz, DMSO-d₆) δ 12.41 (br s, 1H), 8.48 (s, 1H), 7.60 (d, J=9.1 Hz, 2H), 7.23 (br d, J=9 Hz, 2H), 4.37-4.25 (m, 1H), 3.60-3.51 (m, 1H), 3.51-3.43 (m, 1H), 2.24-2.11 (m, 1H), 1.99-1.82 (m, 3H).

### Preparation P3

### Methyl 4-(4-amino-1,3-thiazol-2-yl)cyclohexane-1-carboxylate (P3)

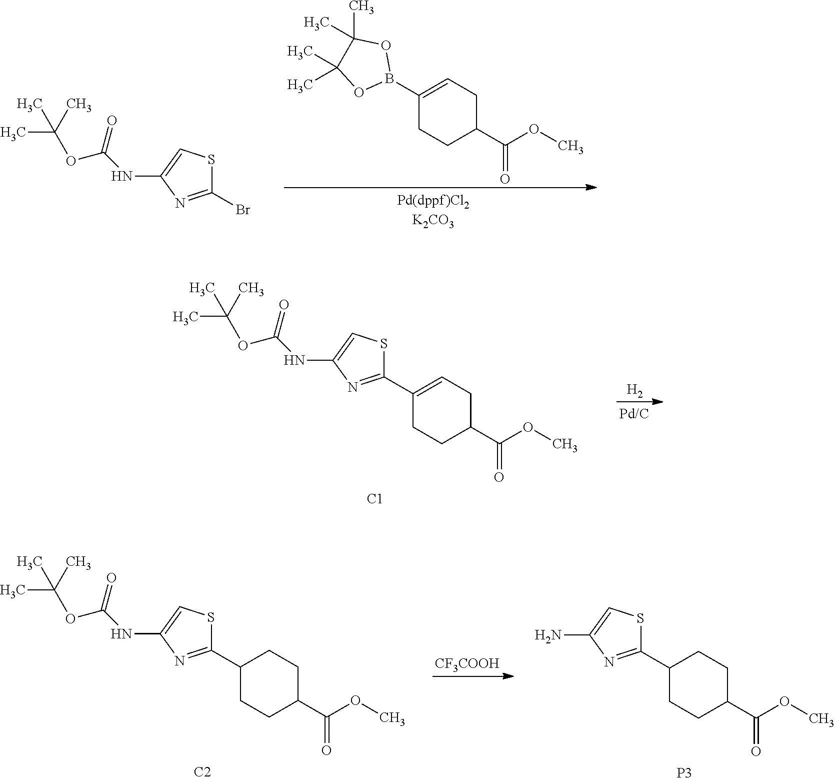

### Step 1. Synthesis of methyl 4-{4-[(tert-butoxycarbonyl)amino]-1,3-thiazol-2-yl}cyclohex-3-ene-1-carboxylate (C1)

[1,1′-Bis(diphenylphosphino)ferrocene]dichloropalladium(II) (83.9 mg, 0.115 mmol) was added to a solution of tert-butyl (2-bromo-1,3-thiazol-4-yl)carbamate (400 mg, 1.43 mmol), methyl 4-(4,4,5,5-tetramethyl-1,3,2-dioxaborolan-2-yl)cyclohex-3-ene-1-carboxylate (419 mg, 1.57 mmol), and potassium carbonate (396 mg, 2.87 mmol) in a mixture of 1,4-dioxane (10 mL) and water (2 mL), whereupon the reaction mixture was stirred at 85° C. for 2 hours. Solvent was then removed in vacuo; the residue was purified by silica gel chromatography (Gradient: 20% to 40% ethyl acetate in petroleum ether), providing C1 as a yellow oil. Yield: 402 mg, 1.19 mmol, 83%. LCMS m/z 339.1 [M+H]⁺. ¹H NMR (400 MHz, CDCl₃) δ 7.43 (br s, 1H), 7.08 (br s, 1H), 6.67-6.59 (m, 1H), 3.71 (s, 3H), 2.72-2.59 (m, 2H), 2.56-2.39 (m, 3H), 2.23-2.12 (m, 1H), 1.90-1.76 (m, 1H), 1.51 (s, 9H).

### Step 2. Synthesis of methyl 4-{4-[(tert-butoxycarbonyl)amino]-1,3-thiazol-2-yl}cyclohexane-1-carboxylate (C2)

Palladium on carbon (100 mg) was added to a solution of C1 (400 mg, 1.18 mmol) in methanol (15 mL). After the reaction mixture had been hydrogenated at 40° C. for six days, the catalyst was removed via filtration; the filtrate was concentrated in vacuo to provide C2 as a yellow oil (400 mg), which was progressed directly to the following step. This material was assumed to be a mixture of cis and trans isomers. LCMS m/z 341.2 [M+H]⁺. ¹H NMR (400 MHz, CDCl₃), characteristic peaks, integrations are approximate: δ 7.47 (br s, 1H), 7.07 (br s, 1H), 3.69 (s, 3H), [3.07-2.95 (m) and 2.93-2.82 (m), total 1H], 2.16-2.05 (m, 2H), 2.02-1.79 (m, 4H), 1.77-1.64 (m, 2H), 1.51 (s, 9H).

### Step 3. Synthesis of methyl 4-(4-amino-1,3-thiazol-2-yl)cyclohexane-1-carboxylate (P3)

Trifluoroacetic acid (2 mL) was added to a solution of C2 (from the previous step; 400 mg, ≤1.17 mmol) in dichloromethane (8 mL). After the reaction mixture had been stirred at 25° C. for 2 hours, LCMS analysis indicated conversion to P3: LCMS m/z 241.1 [M+H]⁺. The reaction mixture was diluted with water (30 mL) and saturated aqueous sodium bicarbonate solution (15 mL), then extracted with dichloromethane (3×30 mL). The combined organic layers were dried over sodium sulfate, filtered, and concentrated in vacuo, affording P3 as a yellow oil. This material was used without additional purification. Yield: 260 mg, 1.08 mmol, 92% over 2 steps.

### Preparation P4

### Methyl 1-(5-amino-3-fluoropyridin-2-yl)pyrrolidine-3-carboxylate (P4)

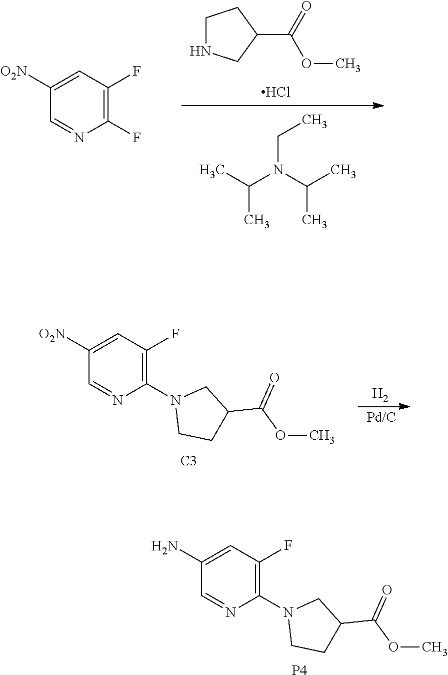

### Step 1. Synthesis of methyl 1-(3-fluoro-5-nitropyridin-2-yl)pyrrolidine-3-carboxylate (C3)

N,N-Diisopropylethylamine (604 mg, 4.67 mmol) was added to a mixture of 2,3-difluoro-5-nitropyridine (500 mg, 3.12 mmol) and methyl pyrrolidine-3-carboxylate, hydrochloride salt (517 mg, 3.12 mmol) in acetonitrile (10 mL). After the reaction mixture had been stirred at 20° C. for 16 hours, it was concentrated in vacuo and purified via silica gel chromatography (Gradient: 0% to 15% ethyl acetate in petroleum ether), affording C3 as a yellow solid. Yield: 742 mg, 2.76 mmol, 88%. LCMS m/z 270.1 [M+H]⁺.

### Step 2. Synthesis of methyl 1-(5-amino-3-fluoropyridin-2-yl)pyrrolidine-3-carboxylate (P4)

A mixture of C3 (742 mg, 2.76 mmol) and palladium on carbon (10%, 80 mg) in methanol (30 mL) was stirred under a hydrogen atmosphere at 20° C. for 3 hours. Filtration, followed by concentration of the filtrate in vacuo, provided P4 as a solid. Yield: 420 mg, 1.76 mmol, 64%. LCMS m/z 240.1 [M+H]⁺.

### Preparation of P5, P6, P7, and P8

Methyl (1S,3R)-3-(5-amino-3-fluoropyridin-2-yl)cyclopentane-1-carboxylate, hydrochloride salt;
Methyl (1R,3R)-3-(5-amino-3-fluoropyridin-2-yl)cyclopentane-1-carboxylate, hydrochloride salt;
Methyl (1S,3S)-3-(5-amino-3-fluoropyridin-2-yl)cyclopentane-1-carboxylate, hydrochloride salt; and
Methyl (1R,3S)-3-(5-amino-3-fluoropyridin-2-yl)cyclopentane-1-carboxylate, hydrochloride salt [P5, from C6 (DIAST-1)], [P6, from C7 (DIAST-2)], [P7, from C8 (DIAST-3)], and [P8, from C9 (DIAST-4)]

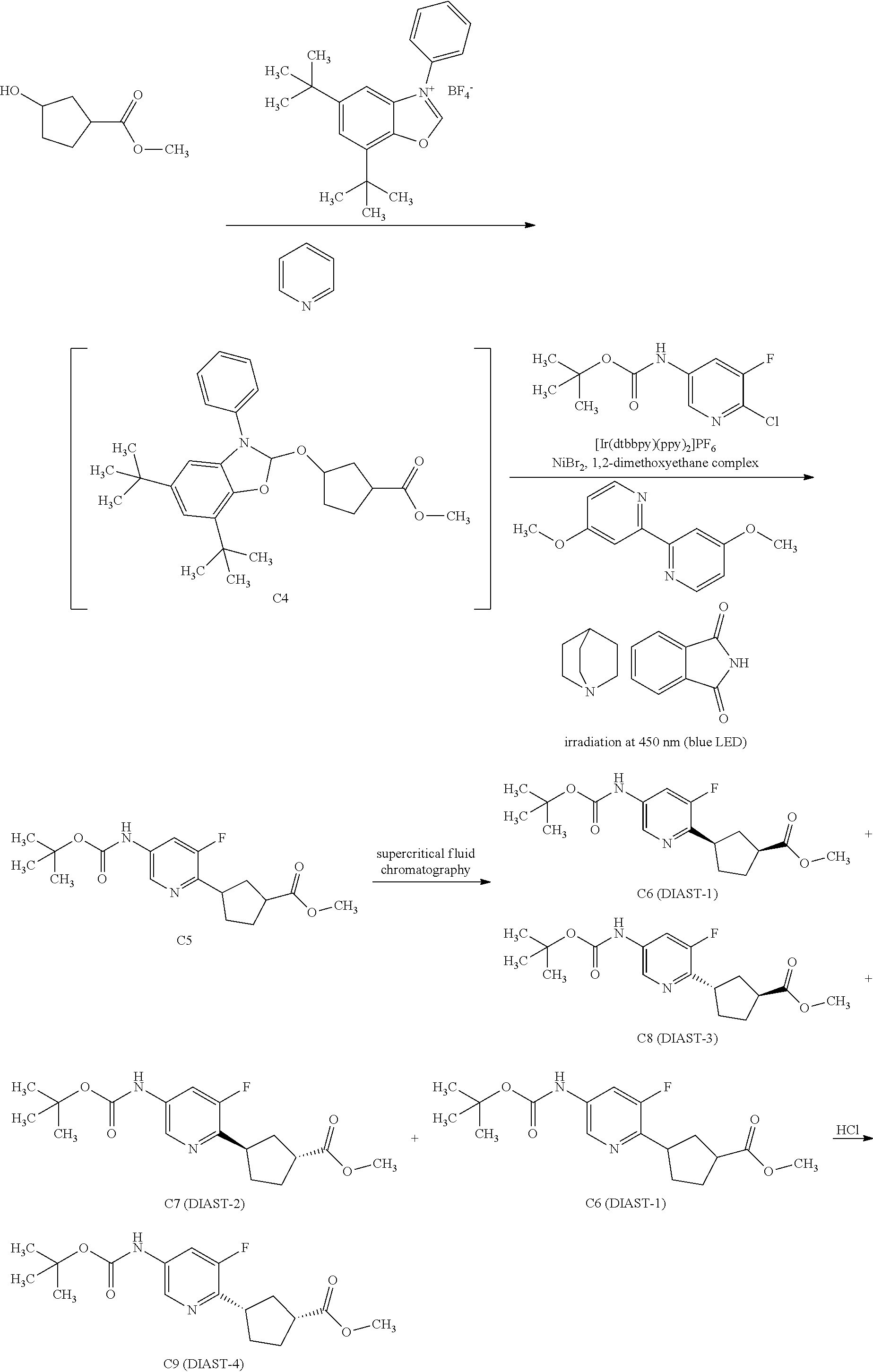

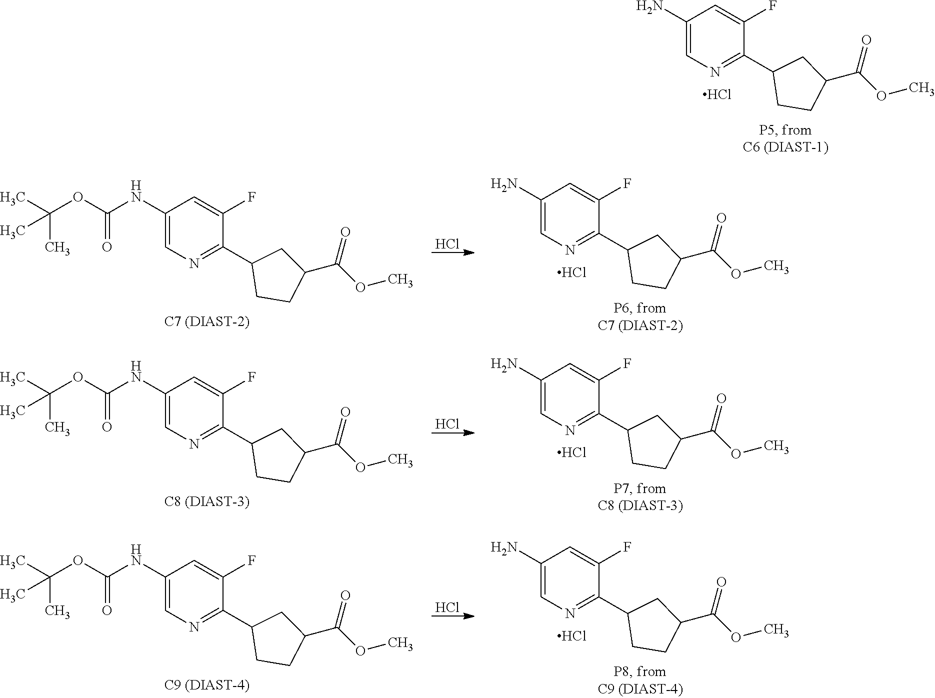

### Step 1. Synthesis of methyl 3-{5-[(tert-butoxycarbonyl)amino]-3-fluoropyridin-2-yl}cyclopentane-1-carboxylate (C5)

A vial containing methyl 3-hydroxycyclopentane-1-carboxylate (500 mg, 3.47 mmol) and 5,7-di-tert-butyl-3-phenylbenzo[d]oxazol-3-ium tetrafluoroborate (1.37 g, 3.47 mmol) in methyl tert-butyl ether (12 mL) was evacuated and charged with nitrogen. This evacuation cycle was repeated, whereupon the reaction mixture was stirred at 15° C. for 5 minutes before being treated with pyridine (0.279 mL, 3.45 mmol). Stirring was continued for 20 minutes to provide a solution containing methyl 3-[(5,7-di-tert-butyl-3-phenyl-2,3-dihydro-1,3-benzoxazol-2-yl)oxy]cyclopentane-1-carboxylate (C4).

The solution of C₄ was added, via a syringe fitted with a filter, to a mixture of tert-butyl (6-chloro-5-fluoropyridin-3-yl)carbamate (329 mg, 1.33 mmol), [4,4′-bis(1,1-dimethylethyl)-2,2′-bipyridine-N1,N1′]bis[2-(2-pyridinyl-N)phenyl-C]iridium(III) hexafluorophosphate (MFCD27952560; [lr(dtbbpy)(ppy)₂]PF₆; 36.6 mg, 40.0 μmol), nickel(II) bromide 1,2-dimethoxyethane complex (41.2 mg, 0.133 mmol), 4,4′-dimethoxy-2,2′-bipyridine (28.8 mg, 0.133 mmol), 1-azabicyclo[2.2.2]octane (260 mg, 2.34 mmol), and 1H-isoindole-1,3(2H)-dione (49.1 mg, 0.334 mmol), in N,N-dimethylacetamide (13.3 mL) (this mixture was assembled in a glove box). The reaction vessel was then irradiated at 15° C. for two days with 100% blue LED light (450 nm), under a fan speed of 5000 rpm and stir rate of 1000 rpm, whereupon the reaction mixture was filtered. The filtrate was subjected to reversed-phase HPLC (Column: WePure Biotech XP tC18, 40×150 mm, 7 μm; Mobile phase A: water containing 0.05% ammonium hydroxide and 10 mM ammonium bicarbonate; Mobile phase B: acetonitrile; Gradient: 38% to 78% B; Flow rate: 60 mL/minute), affording C₅ as a yellow gum. Yield: 247 mg, 0.730 mmol, 55%. LCMS m/z 339.3 [M+H]⁺.

### Step 2. Isolation of methyl (1S,3R)-3-{5-[(tert-butoxycarbonyl)amino]-3-fluoropyridin-2-yl}cyclopentane-1-carboxylate, methyl (1R,3R)-3-{5-[(tert-butoxycarbonyl)amino]-3-fluoropyridin-2-yl}cyclopentane-1-carboxylate, methyl (1S,3S)-3-{5-[(tert-butoxycarbonyl)amino]-3-fluoropyridin-2-yl}cyclopentane-1-carboxylate, and methyl (1R,3S)-3-{5-[(tert-butoxycarbonyl)amino]-3-fluoropyridin-2-yl}cyclopentane-1-carboxylate [C6 (DIAST-1), C7 (DIAST-2), C8 (DIAST-3), and C9 (DIAST-4)]

Separation of C₅ (277 mg, 0.819 mmol) into its component diastereomers was carried out via supercritical fluid chromatography [Column: Chiral Technologies Chiralpak IG, 30×250 mm, 10 μm; Mobile phase: 1:1 carbon dioxide/(methanol containing 0.1% ammonium hydroxide); Back pressure: 100 bar; Flow rate: 80 g/minute]. Mixed fractions of the second- and third-eluting isomers were separated using supercritical fluid chromatography [Column: Chiral Technologies Chiralpak AD, 30×250 mm, 10 μm; Mobile phase: 3:2 carbon dioxide/(methanol containing 0.1% ammonium hydroxide); Back pressure: 100 bar; Flow rate: 100 g/minute]. The four diastereomers were designated according to their order of elution: C6 (DIAST-1), C7 (DIAST-2), C8 (DIAST-3), and C9 (DIAST-4). All four were obtained as faintly yellow oils.

C6 (DIAST-1)—Yield: 63 mg, 0.19 mmol, 23%. LCMS m/z 339.3 [M+H]⁺. Retention time: 1.99 minutes. Analytical conditions. Column: Chiral Technologies Chiralpak IG-3, 4.6×50 mm, 3 μm; Mobile phase A: carbon dioxide; Mobile phase B: [methanol containing 0.2% (7 M ammonia in methanol)]; Gradient: 5% to 40% B over 2 minutes, then 40% B for 1.2 minutes; Flow rate: 4 mL/minute; Back pressure: 1500 psi.

C7 (DIAST-2)—Yield: 70 mg, 0.21 mmol, 26%. LCMS m/z 339.1 [M+H]⁺. Retention time: 2.14 minutes. Analytical conditions identical to those used for C6 (DIAST-1).

C8 (DIAST-3)—Yield: 64 mg, 0.19 mmol, 23%. LCMS m/z 339.1 [M+H]⁺. Retention time: 2.25 minutes. Analytical conditions identical to those used for C6 (DIAST-1).

C9 (DIAST-4)—Yield: 83 mg, 0.24 mmol, 29%. LCMS m/z 339.3 [M+H]⁺. Retention time: 2.63 minutes. Analytical conditions identical to those used for C6 (DIAST-1).

### Step 4. Synthesis of methyl (1S,3R)-3-(5-amino-3-fluoropyridin-2-yl)cyclopentane-1-carboxylate, hydrochloride salt; methyl (1R,3R)-3-(5-amino-3-fluoropyridin-2-yl)cyclopentane-1-carboxylate, hydrochloride salt; methyl (1S,3S)-3-(5-amino-3-fluoropyridin-2-yl)cyclopentane-1-carboxylate, hydrochloride salt; or methyl (1R,3S)-3-(5-amino-3-fluoropyridin-2-yl)cyclopentane-1-carboxylate, hydrochloride salt [P5, from C6 (DIAST-1)]

A 15° C. solution of C6 (DIAST-1) (63 mg, 0.19 mmol) in dichloromethane (3 mL) was treated with a solution of hydrogen chloride in 1,4-dioxane (2 M; 3 mL, 6 mmol). LCMS analysis indicated formation of P5, from C6 (DIAST-1): LCMS m/z 239.0 [M+H]⁺. After the reaction mixture had been stirred at 15° C. for 19 hours, it was concentrated in vacuo to provide P5, from C6 (DIAST-1) as a yellow gum. Yield: 51 mg, assumed quantitative.

### Step 5. Synthesis of methyl (1S,3R)-3-(5-amino-3-fluoropyridin-2-yl)cyclopentane-1-carboxylate, hydrochloride salt; methyl (1R,3R)-3-(5-amino-3-fluoropyridin-2-yl)cyclopentane-1-carboxylate, hydrochloride salt; methyl (1S,3S)-3-(5-amino-3-fluoropyridin-2-yl)cyclopentane-1-carboxylate, hydrochloride salt; or methyl (1R,3S)-3-(5-amino-3-fluoropyridin-2-yl)cyclopentane-1-carboxylate, hydrochloride salt [P6, from C7 (DIAST-2)]

A solution of hydrogen chloride in 1,4-dioxane (2 M; 5 mL, 10 mmol) was added to C₇ (DIAST-2) (70 mg, 0.21 mmol), and the reaction mixture was stirred at 15° C. for 2 days. LCMS analysis indicated formation of P6, from C7 (DIAST-2): LCMS m/z 239.0 [M+H]⁺. Concentration in vacuo afforded P6, from C7 (DIAST-2) as a yellow gum. Yield: 57 mg, assumed quantitative.

### Step 6. Synthesis of methyl (1S,3R)-3-(5-amino-3-fluoropyridin-2-yl)cyclopentane-1-carboxylate, hydrochloride salt; methyl (1R,3R)-3-(5-amino-3-fluoropyridin-2-yl)cyclopentane-1-carboxylate, hydrochloride salt; methyl (1S,3S)-3-(5-amino-3-fluoropyridin-2-yl)cyclopentane-1-carboxylate, hydrochloride salt; or methyl (1R,3S)-3-(5-amino-3-fluoropyridin-2-yl)cyclopentane-1-carboxylate, hydrochloride salt [P7, from C8 (DIAST-3)]

Compound C8 (DIAST-3) (64 mg, 0.19 mmol) and a solution of hydrogen chloride in 1,4-dioxane (2 M; 5 mL, 10 mmol) were combined at 15° C. After the reaction mixture had been stirred at 15° C. for 16 hours, LCMS analysis indicated conversion to P7, from C8 (DIAST-3): LCMS m/z 239.0 [M+H]⁺. Concentration in vacuo provided P7, from C8 (DIAST-3) as a yellow gum. Yield: 52 mg, assumed quantitative.

### Step 7. Synthesis of methyl (1S,3R)-3-(5-amino-3-fluoropyridin-2-yl)cyclopentane-1-carboxylate, hydrochloride salt; methyl (1R,3R)-3-(5-amino-3-fluoropyridin-2-yl)cyclopentane-1-carboxylate, hydrochloride salt; methyl (1S,3S)-3-(5-amino-3-fluoropyridin-2-yl)cyclopentane-1-carboxylate, hydrochloride salt; or methyl (1R,3S)-3-(5-amino-3-fluoropyridin-2-yl)cyclopentane-1-carboxylate, hydrochloride salt [P8, from C9 (DIAST-4)]

Compound C9 (DIAST-4) (83 mg, 0.22 mmol) and a solution of hydrogen chloride in 1,4-dioxane (2 M; 5 mL, 10 mmol) were combined at 15° C. After 16 hours at this temperature, LCMS analysis indicated conversion to P8, from C9 (DIAST-4): LCMS m/z 238.9 [M+H]⁺. The reaction mixture was stirred at 15° C. for a total of 2 days, whereupon concentration in vacuo afforded P8, from C9 (DIAST-4) as a yellow gum. Yield: 67 mg, assumed quantitative.

### Examples 1 and 2

### (1S,4s)-4-{4-[(1-{[4-(Trifluoromethyl)phenyl]carbamoyl}-D-prolyl)amino]-1,3-thiazol-2-yl}cyclohexane-1-carboxylic acid and (1R,4r)-4-{4-[(1-{[4-(Trifluoromethyl)phenyl]carbamoyl}-D-prolyl)amino]-1,3-thiazol-2-yl}cyclohexane-1-carboxylic acid [1 (DIAST-1) and 2 (DIAST-2)]

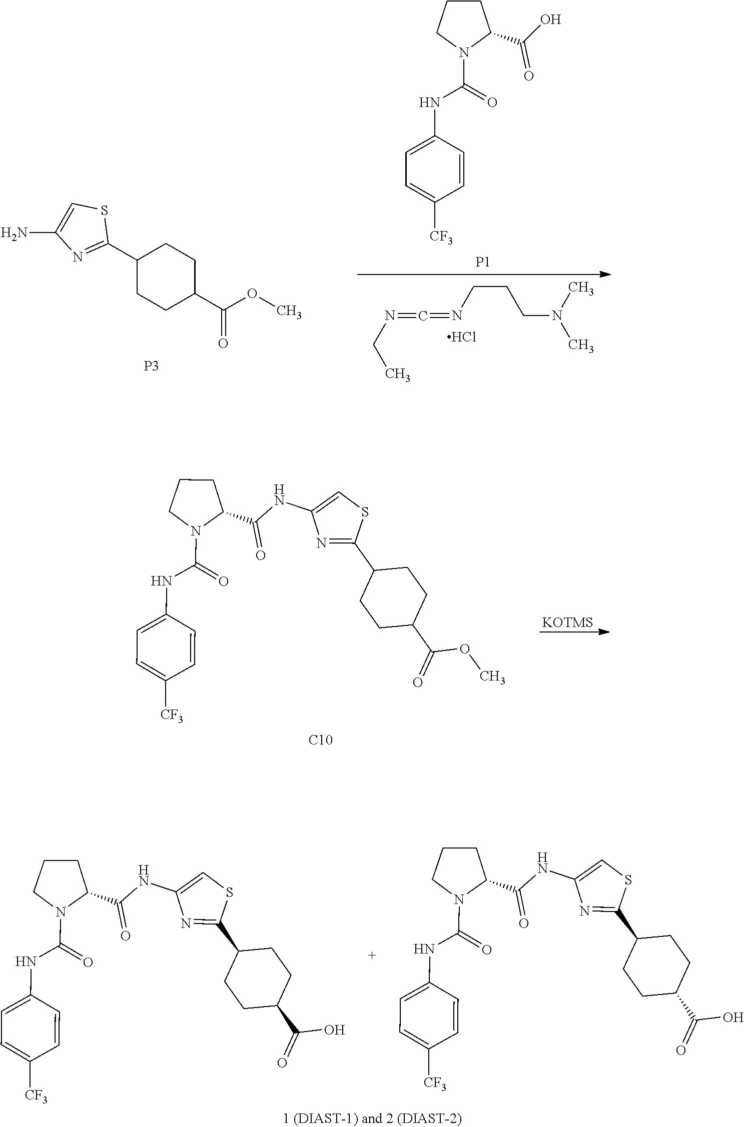

### Step 1. Synthesis of methyl 4-{4-[(1-{[4-(trifluoromethyl)phenyl]carbamoyl}-D-prolyl)amino]-1,3-thiazol-2-yl}cyclohexane-1-carboxylate (C10)

To a solution of P1 (120 mg, 0.397 mmol) and 1-[3-(dimethylamino)propyl]-3-ethylcarbodiimide hydrochloride (152 mg, 0.793 mmol) in dichloromethane (15 mL) was added P3 (95.4 mg, 0.397 mmol), whereupon the reaction mixture was stirred at 25° C. for 16 hours. After removal of solvent in vacuo, the residue was purified by silica gel chromatography (Gradient: 30% to 70% ethyl acetate in petroleum ether) to provide C₁₀ as a white solid. Yield: 175 mg, 0.334 mmol, 84%. LCMS m/z 525.2 [M+H]⁺. ¹H NMR (400 MHz, CDCl₃), characteristic peaks, integrations are approximate: δ [10.06 (br s) and 9.93 (br s), total 1H], 7.62-7.49 (m, 5H), [6.78 (br s) and 6.71 (br s), total 1H], 4.79-4.70 (m, 1H), 3.88-3.73 (m, 1H), 3.69 (s, 3H), 3.57-3.46 (m, 1H), [3.12-3.02 (m) and 2.99-2.88 (m), total 1H], 2.48-2.31 (m, 2H), 1.65-1.47 (m, 3H).

### Step 2. Synthesis of (1S,4s)-4-{4-[(1-{[4-(trifluoromethyl)phenyl]carbamoyl}-D-prolyl)amino]-1,3-thiazol-2-yl}cyclohexane-1-carboxylic acid and (1R,4r)-4-{4-[(1-{[4-(trifluoromethyl)phenyl]carbamoyl}-D-prolyl)amino]-1,3-thiazol-2-yl}cyclohexane-1-carboxylic acid [1 (DIAST-1) and 2 (DIAST-2)]

Potassium trimethylsilanolate (313 mg, 2.44 mmol) was added to a solution of C₁₀ (160 mg, 0.305 mmol) in tetrahydrofuran (10 mL), and the reaction mixture was stirred at 20° C. for 30 minutes. After solvent had been removed under reduced pressure, the residue was dissolved in water (30 mL) and the pH was adjusted to 5 by addition of 2 M hydrochloric acid. The resulting mixture was extracted with ethyl acetate (2×40 mL); the combined organic layers were dried over sodium sulfate, filtered, and concentrated in vacuo. Silica gel chromatography (Gradient: 0% to 8% methanol in dichloromethane) provided a white solid (110 mg), which was separated into its component diastereomers via supercritical fluid chromatography {Column: Chiral Technologies Chiralpak AD, 30×250 mm, 10 μm; Mobile phase: 55:45 carbon dioxide/[methanol containing 0.2% (7 M ammonia in methanol)]; Back pressure: 100 bar; Flow rate: 70 g/minute}, providing (1S,4s)-4-{4-[(1-{[4-(trifluoromethyl)phenyl]carbamoyl}-D-prolyl)amino]-1,3-thiazol-2-yl}cyclohexane-1-carboxylic acid and (1R,4r)-4-{4-[(1-{[4-(trifluoromethyl)phenyl]carbamoyl}-D-prolyl)amino]-1,3-thiazol-2-yl}cyclohexane-1-carboxylic acid. The first-eluting diastereomer was designated as 1 (DIAST-1) and the second-eluting diastereomer as 2 (DIAST-2). One of Compounds 1 and 2 is (1S,4s)-4-{4-[(1-{[4-(trifluoromethyl)phenyl]carbamoyl}-D-prolyl)amino]-1,3-thiazol-2-yl}cyclohexane-1-carboxylic acid and other is (1R,4r)-4-{4-[(1-{[4-(trifluoromethyl)phenyl]carbamoyl}-D-prolyl)amino]-1,3-thiazol-2-yl}cyclohexane-1-carboxylic acid.

1 (DIAST-1)—Yield: 5.5 mg, 11 μmol, 4%. LCMS m/z 511.1 [M+H]⁺. ¹H NMR (400 MHz, DMSO-d₆), aliphatic integrations are approximate: δ 10.98 (br s, 1H), 8.68 (s, 1H), 7.65 (AB quartet, J_{AB}=8.5 Hz, Δv_{AB}=68.4 Hz, 4H), 7.37 (s, 1H), 4.55 (dd, J=8.3, 3.9 Hz, 1H), 3.71-3.60 (m, 1H), 3.60-3.49 (m, 1H), 3.08-2.96 (m, 1H), 2.23-2.10 (m, 1H), 2.07-1.70 (m, 10H), 1.69-1.54 (m, 2H).

2 (DIAST-2)—Yield: 46.9 mg, 91.9 μmol, 30%. LCMS m/z 511.1 [M+H]⁺. ¹H NMR (400 MHz, DMSO-d₆) δ 10.96 (br s, 1H), 8.68 (s, 1H), 7.65 (AB quartet, J_{AB}=8.6 Hz, Δv_{AB}=67.4 Hz, 4H), 7.36 (s, 1H), 4.55 (dd, J=8.4, 3.9 Hz, 1H), 3.70-3.60 (m, 1H), 3.59-3.49 (m, 1H), 2.95-2.84 (m, 1H), 2.26-2.12 (m, 2H), 2.12-2.04 (m, 2H), 2.04-1.82 (m, 5H), 1.57-1.38 (m, 4H).

### Examples 3 and 4

### (1S,4s)-4-{4-[(1-{[4-(Trifluoromethoxy)phenyl]carbamoyl}-D-prolyl)amino]-1,3-thiazol-2-yl}cyclohexane-1-carboxylic acid and (1R,4r)-4-{4-[(1-{[4-(Trifluoromethoxy)phenyl]carbamoyl}-D-prolyl)amino]-1,3-thiazol-2-yl}cyclohexane-1-carboxylic acid [3 (DIAST-1) and 4 (DIAST-2)]

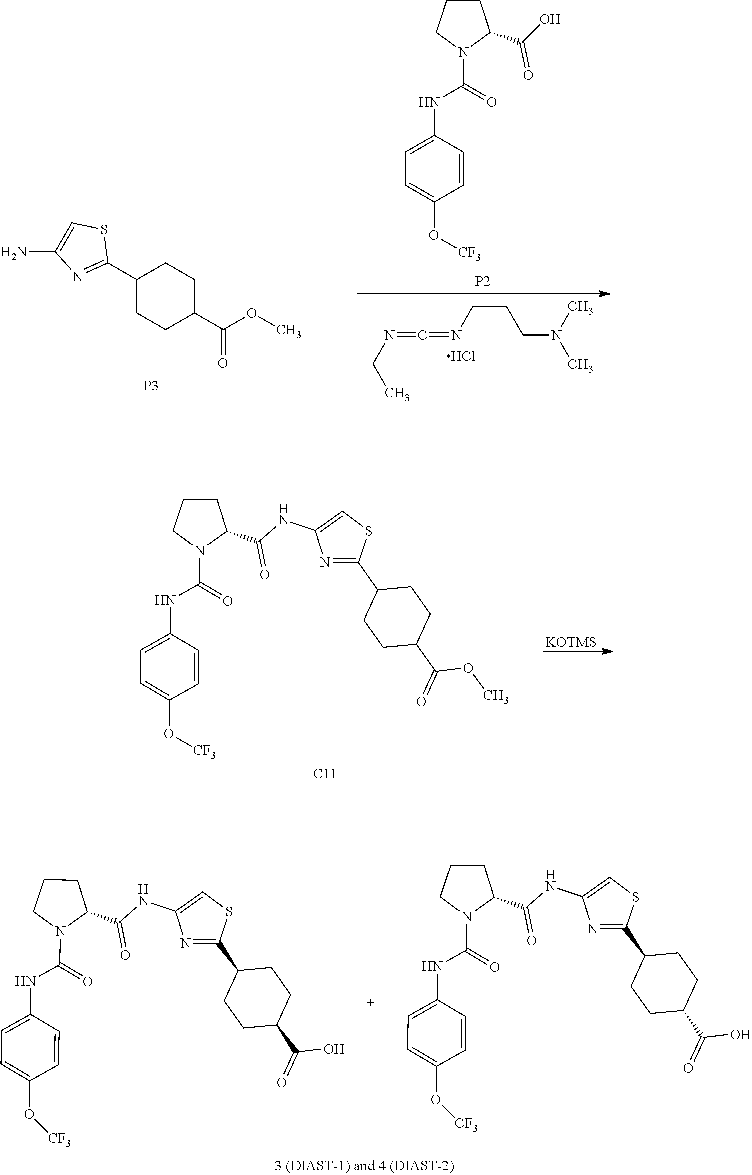

### Step 1. Synthesis of methyl 4-{4-[(1-{[4-(trifluoromethoxy)phenyl]carbamoyl}-D-prolyl)amino]-1,3-thiazol-2-yl}cyclohexane-1-carboxylate (C11)

To a solution of P2 (120 mg, 0.377 mmol) and 1-[3-(dimethylamino)propyl]-3-ethylcarbodiimide hydrochloride (145 mg, 0.756 mmol) in dichloromethane (15 mL) was added P3 (90.6 mg, 0.377 mmol). After the reaction mixture had been stirred at 25° C. for 16 hours, it was concentrated in vacuo; the residue was purified using silica gel chromatography (Gradient: 30% to 70% ethyl acetate in petroleum ether) to provide C₁₁ as a white solid. Yield: 170 mg, 0.314 mmol, 83%. LCMS m/z 541.2 [M+H]⁺. ¹H NMR (400 MHz, CDCl₃), characteristic peaks, integrations are approximate: δ [9.98 (br s) and 9.88 (br s), 1H], [7.55 (s) and 7.52 (s), total 1H], 7.45 (d, J=9.0 Hz, 2H), 7.15 (br d, J=8.5 Hz, 2H), [6.59 (br s) and 6.54 (br s), total 1H], 4.77-4.68 (m, 1H), 3.82-3.68 (m, 1H), 3.69 (s, 3H), 3.54-3.43 (m, 1H), [3.12-3.00 (m) and 2.99-2.86 (m), total 1H], 2.50-2.30 (m, 2H), 1.65-1.48 (m, 3H).

### Step 2. Synthesis of (1S,4s)-4-{4-[(1-{[4-(trifluoromethoxy)phenyl]carbamoyl}-D-prolyl)amino]-1,3-thiazol-2-yl}cyclohexane-1-carboxylic acid and (1R,4r)-4-{4-[(1-{[4-(trifluoromethoxy)phenyl]carbamoyl}-D-prolyl)amino]-1,3-thiazol-2-yl}cyclohexane-1-carboxylic acid [3 (DIAST-1) and 4 (DIAST-2)]

Potassium trimethylsilanolate (323 mg, 2.52 mmol) was added to a solution of C₁₁ (170 mg, 0.314 mmol) in tetrahydrofuran (10 mL), whereupon the reaction mixture was stirred at 20° C. for 16 hours. After concentration under reduced pressure, the residue was dissolved in water (30 mL), adjusted to pH 5 by addition of 2 M hydrochloric acid, and extracted with ethyl acetate (2×40 mL). The combined organic layers were dried over sodium sulfate, filtered, and concentrated in vacuo. Silica gel chromatography (Gradient: 0% to 8% methanol in dichloromethane) afforded a white solid (120 mg). A portion of this material (100 mg) was separated into its component diastereomers using supercritical fluid chromatography {Column: Chiral Technologies Chiralpak AD, 30×250 mm, 10 μm; Mobile phase: 1:1 carbon dioxide/[methanol containing 0.2% (7 M ammonia in methanol)]; Flow rate: 80 g/minute}, providing (1S,4s)-4-{4-[(1-{[4-(trifluoromethoxy)phenyl]carbamoyl}-D-prolyl)amino]-1,3-thiazol-2-yl}cyclohexane-1-carboxylic acid and (1R,4r)-4-{4-[(1-{[4-(trifluoromethoxy)phenyl]carbamoyl}-D-prolyl)amino]-1,3-thiazol-2-yl}cyclohexane-1-carboxylic acid. The first-eluting diastereomer was designated as 3 (DIAST-1), and the second-eluting as 4 (DIAST-2). One of Compounds 3 and 4 is (1S,4s)-4-{4-[(1-{[4-(trifluoromethoxy)phenyl]carbamoyl}-D-prolyl)amino]-1,3-thiazol-2-yl}cyclohexane-1-carboxylic acid, and the other is (1R,4r)-4-{4-[(1-{[4-(trifluoromethoxy)phenyl]carbamoyl}-D-prolyl)amino]-1,3-thiazol-2-yl}cyclohexane-1-carboxylic acid.

3 (DIAST-1)—Adjusted yield: 14.2 mg, 27.0 μmol, 10%. LCMS m/z 527.2 [M+H]⁺. ¹H NMR (400 MHz, DMSO-d₆), aliphatic integrations are approximate: δ 10.94 (br s, 1H), 8.47 (s, 1H), 7.60 (d, J=9.1 Hz, 2H), 7.36 (s, 1H), 7.22 (br d, J=8.6 Hz, 2H), 4.53 (dd, J=8.3, 3.9 Hz, 1H), 3.68-3.58 (m, 1H), 3.56-3.46 (m, 1H), 3.07-2.96 (m, 1H), 2.22-2.08 (m, 1H), 2.05-1.70 (m, 1OH), 1.67-1.53 (m, 2H).

4 (DIAST-2)—Adjusted yield: 45 mg, 85 μmol, 32%. LCMS m/z 527.2 [M+H]⁺. ¹H NMR (400 MHz, DMSO-d₆) δ 10.93 (br s, 1H), 8.48 (s, 1H), 7.60 (d, J=9.1 Hz, 2H), 7.36 (s, 1H), 7.22 (d, J=8.6 Hz, 2H), 4.53 (dd, J=8.2, 3.9 Hz, 1H), 3.68-3.58 (m, 1H), 3.56-3.46 (m, 1H), 2.95-2.84 (m, 1H), 2.26-1.81 (m, 9H), 1.56-1.38 (m, 4H).

### Examples 5 and 6

### (3S)-1-{3-Fluoro-5-[(1-{[4-(trifluoromethoxy)phenyl]carbamoyl}-D-prolyl)amino]pyridin-2-yl}pyrrolidine-3-carboxylic acid and (3R)-1-{3-Fluoro-5-[(1-{[4-(trifluoromethoxy)phenyl]carbamoyl}-D-prolyl)amino]pyridin-2-yl}pyrrolidine-3-carboxylic acid [5 (DIAST-1) and 6 (DIAST-2)]

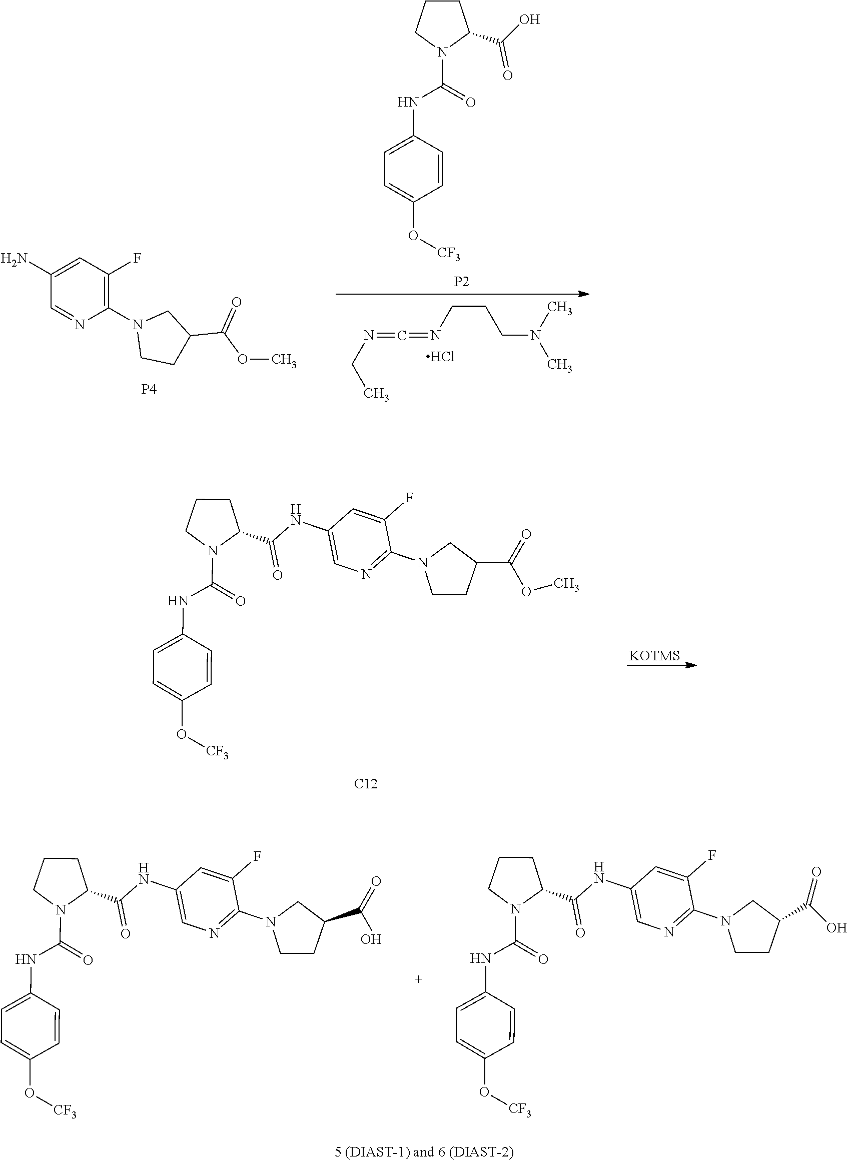

### Step 1. Synthesis of methyl 1-{3-fluoro-5-[(1-{[4-(trifluoromethoxy)phenyl]carbamoyl}-D-prolyl)amino]pyridin-2-yl}pyrrolidine-3-carboxylate (C12)

To a solution of P4 (100 mg, 0.418 mmol) and P2 (200 mg, 0.628 mmol) in dichloromethane (10 mL) was added 1-[3-(dimethylamino)propyl]-3-ethylcarbodiimide hydrochloride (481 mg, 2.51 mmol), whereupon the reaction mixture was stirred at 25° C. for 16 hours. It was then diluted with dichloromethane (20 mL), washed with saturated aqueous sodium chloride solution (2×15 mL), and concentrated in vacuo. Silica gel chromatography (Gradient: 0% to 5% methanol in dichloromethane) provided C₁₂ as a white solid. Yield: 140 mg, 0.260 mmol, 62%. LCMS m/z 540.2 [M+H]⁺.

### Step 2. Synthesis of (3S)-1-{3-fluoro-5-[(1-{[4-(trifluoromethoxy)phenyl]carbamoyl}-D-prolyl)amino]pyridin-2-yl}pyrrolidine-3-carboxylic acid and (3R)-1-{3-fluoro-5-[(1-{[4-(trifluoromethoxy)phenyl]carbamoyl}-D-prolyl)amino]pyridin-2-yl}pyrrolidine-3-carboxylic acid [5 (DIAST-1) and 6 (DIAST-2)]

A mixture of C12 (140 mg, 0.260 mmol) and potassium trimethylsilanolate (117 mg, 0.912 mmol) in tetrahydrofuran (10 mL) was stirred at 24° C. for two hours, whereupon the reaction mixture was acidified to pH 1 by addition of 3 M hydrochloric acid. The resulting mixture was extracted twice with ethyl acetate (30 mL); the combined organic layers were concentrated in vacuo and purified via silica gel chromatography (Gradient: 0% to 15% methanol in dichloromethane), affording a white solid (60 mg). This material was separated into its component diastereomers via supercritical fluid chromatography {Column: Chiral Technologies Chiralpak AD, 30×250 mm, 10 μm; Mobile phase: 1:1 carbon dioxide/[methanol containing 0.2% (7 M ammonia in methanol)]; Flow rate: 120 g/minute}, affording (3S)-1-{3-fluoro-5-[(1-{[4-(trifluoromethoxy)phenyl]carbamoyl}-D-prolyl)amino]pyridin-2-yl}pyrrolidine-3-carboxylic acid and (3R)-1-{3-fluoro-5-[(1-{[4-(trifluoromethoxy)phenyl]carbamoyl}-D-prolyl)amino]pyridin-2-yl}pyrrolidine-3-carboxylic acid. The first-eluting diastereomer was designated as 5 (DIAST-1) and the second-eluting diastereomer as 6 (DIAST-2). One of Compounds 5 and 6 is (3S)-1-{3-fluoro-5-[(1-{[4-(trifluoromethoxy)phenyl]carbamoyl}-D-prolyl)amino]pyridin-2-yl}pyrrolidine-3-carboxylic acid, and the other is (3R)-1-{3-fluoro-5-[(1-{[4-(trifluoromethoxy)phenyl]carbamoyl}-D-prolyl)amino]pyridin-2-yl}pyrrolidine-3-carboxylic acid.

5 (DIAST-1)—Yield: 21.5 mg, 40.9 μmol, 16%. LCMS m/z 526.1 [M+H]⁺. ¹H NMR (400 MHz, DMSO-d₆) δ 10.09-9.98 (m, 1H), 8.52 (br s, 1H), 8.07 (br s, 1H), 7.76 (dd, J=15.1, 2.1 Hz, 1H), 7.62 (d, J=9.1 Hz, 2H), 7.22 (br d, J=9 Hz, 2H), 4.46-4.38 (m, 1H), 3.71-3.59 (m, 3H), 3.59-3.44 (m, 3H), 3.09-2.97 (m, 1H), 2.24-1.85 (m, 6H).

6 (DIAST-2)—Yield: 30.7 mg, 58.4 μmol, 22%. LCMS m/z 526.2 [M+H]⁺. ¹H NMR (400 MHz, DMSO-d₆) δ 10.02 (br s, 1H), 8.51 (br s, 1H), 8.07 (br s, 1H), 7.77 (dd, J=15.0, 2.1 Hz, 1H), 7.62 (d, J=9.1 Hz, 2H), 7.22 (br d, J=9 Hz, 2H), 4.46-4.38 (m, 1H), 3.71-3.59 (m, 3H), 3.59-3.45 (m, 3H), 3.11-3.00 (m, 1H), 2.23-1.85 (m, 6H).

### Example 7

### (1S,3R)-3-{3-Fluoro-5-[(1-{[4-(trifluoromethyl)phenyl]carbamoyl}-D-prolyl)amino]pyridin-2-yl}cyclopentane-1-carboxylic acid, (1R,3R)-3-{3-Fluoro-5-[(1-{[4-(trifluoromethyl)phenyl]carbamoyl}-D-prolyl)amino]pyridin-2-yl}cyclopentane-1-carboxylic acid, (1S,3S)-3-{3-Fluoro-5-[(1-{[4-(trifluoromethyl)phenyl]carbamoyl}-D-prolyl)amino]pyridin-2-yl}cyclopentane-1-carboxylic acid, or (1R,3S)-3-{3-Fluoro-5-[(1-{[4-(trifluoromethyl)phenyl]carbamoyl}-D-prolyl)amino]pyridin-2-yl}cyclopentane-1-carboxylic acid [7, from C6 (DIAST-1)]

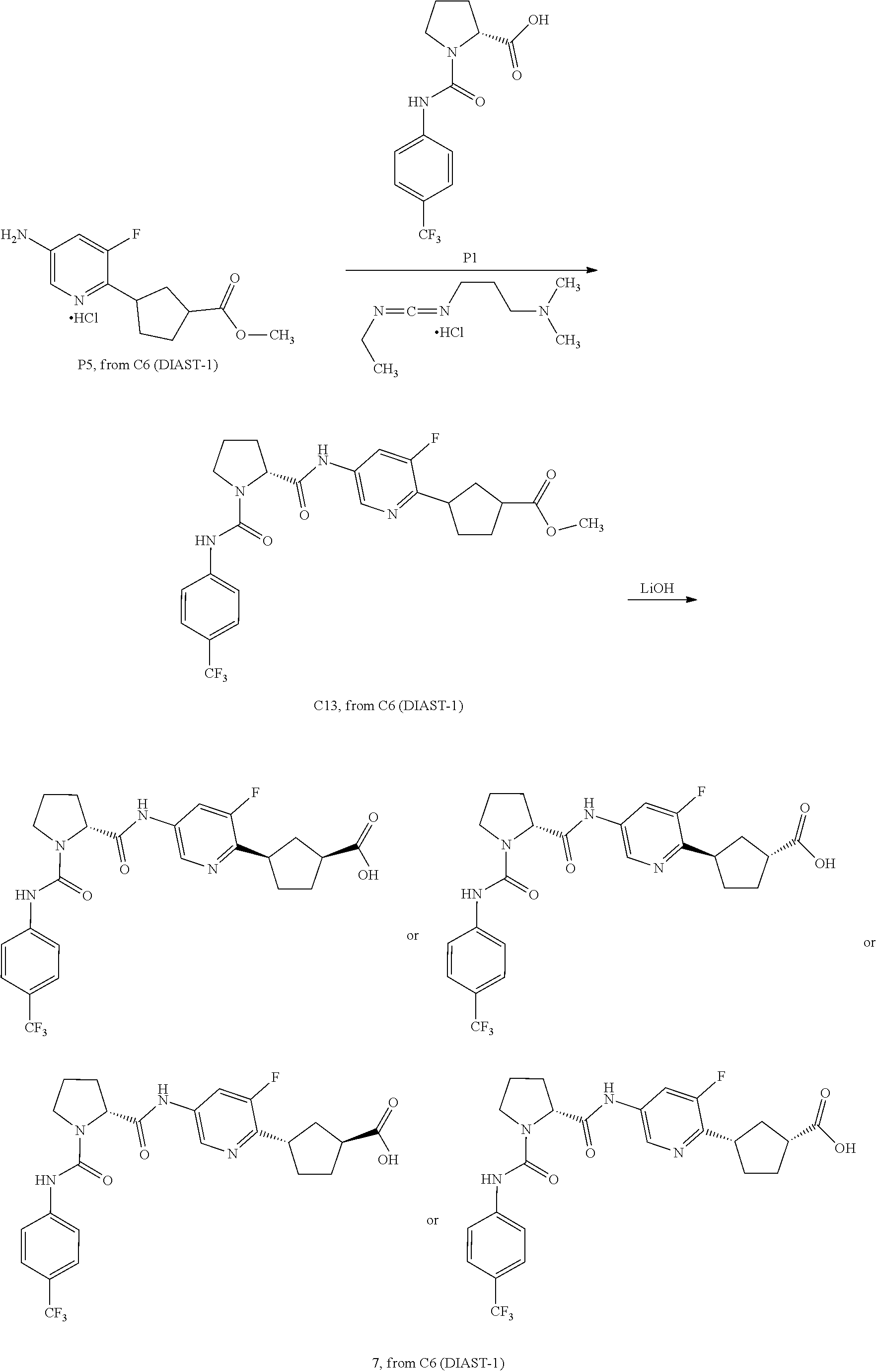

### Step 1. Synthesis of methyl 3-{3-fluoro-5-[(1-{[4-(trifluoromethyl)phenyl]carbamoyl}-D-prolyl)amino]pyridin-2-yl}cyclopentane-1-carboxylate [C13, from C6 (DIAST-1)]

1-[3-(Dimethylamino)propyl]-3-ethylcarbodiimide hydrochloride (17.7 mg, 92.3 μmol) was added to a 15° C. solution of P1 (23.3 mg, 77.1 μmol) and P5, from C6 (DIA ST-1) (21.0 mg, 76.4 μmol) in dichloromethane (0.55 mL), whereupon the reaction mixture was stirred at 15° C. for 16 hours. Purification via thin-layer chromatography (Eluant: ethyl acetate) afforded C13, from C6 (DIAST-1) as a colorless gum. Yield: 25 mg, 48 μmol, 63%. LCMS m/z 523.1 [M+H]⁺.

### Step 2. Synthesis of (1S,3R)-3-{3-fluoro-5-[(1-{[4-(trifluoromethyl)phenyl]carbamoyl}-D-prolyl)amino]pyridin-2-yl}cyclopentane-1-carboxylic acid, (1R,3R)-3-{3-fluoro-5-[(1-{[4-(trifluoromethyl)phenyl]carbamoyl}-D-prolyl)amino]pyridin-2-yl}cyclopentane-1-carboxylic acid, (1S,3S)-3-{3-fluoro-5-[(1-{[4-(trifluoromethyl)phenyl]carbamoyl}-D-prolyl)amino]pyridin-2-yl}cyclopentane-1-carboxylic acid, or (1R,3S)-3-{3-fluoro-5-[(1-{[4-(trifluoromethyl)phenyl]carbamoyl}-D-prolyl)amino]pyridin-2-yl}cyclopentane-1-carboxylic acid [7, from C6 (DIAST-1)]

Lithium hydroxide monohydrate (18.1 mg, 0.431 mmol) was added to a 15° C. solution of C13, from C6 (DIAST-1) (25 mg, 48 μmol) in tetrahydrofuran (1.0 mL). After the reaction mixture had been stirred at 15° C. for 16 hours, it was acidified to pH 6 by addition of hydrochloric acid (1 M; 0.7 mL, 0.7 mmol). Reversed-phase HPLC (Column: WePure Biotech XP tC18, 40×150 mm, 7 μm; Mobile phase A: water containing 0.05% ammonium hydroxide and 10 mM ammonium bicarbonate; Mobile phase B: acetonitrile; Gradient: 7% to 47% B; Flow rate: 60 mL/minute), afforded (1S,3R)-3-{3-fluoro-5-[(1-{[4-(trifluoromethyl)phenyl]carbamoyl}-D-prolyl)amino]pyridin-2-yl}cyclopentane-1-carboxylic acid, (1R,3R)-3-{3-fluoro-5-[(1-{[4-(trifluoromethyl)phenyl]carbamoyl}-D-prolyl)amino]pyridin-2-yl}cyclopentane-1-carboxylic acid, (1S,3S)-3-{3-fluoro-5-[(1-{[4-(trifluoromethyl)phenyl]carbamoyl}-D-prolyl)amino]pyridin-2-yl}cyclopentane-1-carboxylic acid, or (1R,3S)-3-{3-fluoro-5-[(1-{[4-(trifluoromethyl)phenyl]carbamoyl}-D-prolyl)amino]pyridin-2-yl}cyclopentane-1-carboxylic acid [7, from C6 (DIAST-1)] as a white solid. Yield: 6 mg, 12 μmol, 25%. LCMS m/z 509.3 [M+H]⁺. ¹H NMR (400 MHz, CDCl₃) δ 10.21 (s, 1H), 8.43 (br d, J=2 Hz, 1H), 8.22 (dd, J=11.3, 2.1 Hz, 1H), 7.56 (AB quartet, J_{AB}=9.0 Hz, Δv_{AB}=11.5 Hz, 4H), 6.66 (s, 1H), 4.80-4.74 (m, 1H), 3.88-3.79 (m, 1H), 3.70-3.62 (m, 1H), 3.54-3.45 (m, 1H), 3.17-3.05 (m, 1H), 2.61-2.51 (m, 1H), 2.39-2.31 (m, 2H), 2.29-2.12 (m, 3H), 2.11-1.95 (m, 2H), 1.91-1.78 (m, 2H).

### Example 8

### (1S,3R)-3-{3-Fluoro-5-[(1-{[4-(trifluoromethyl)phenyl]carbamoyl}-D-prolyl)amino]pyridin-2-yl}cyclopentane-1-carboxylic acid, (1R,3R)-3-{3-Fluoro-5-[(1-{[4-(trifluoromethyl)phenyl]carbamoyl}-D-prolyl)amino]pyridin-2-yl}cyclopentane-1-carboxylic acid, (1S,3S)-3-{3-Fluoro-5-[(1-{[4-(trifluoromethyl)phenyl]carbamoyl}-D-prolyl)amino]pyridin-2-yl}cyclopentane-1-carboxylic acid, or (1R,3S)-3-{3-Fluoro-5-[(1-{[4-(trifluoromethyl)phenyl]carbamoyl}-D-prolyl)amino]pyridin-2-yl}cyclopentane-1-carboxylic acid [8, from C7 (DIAST-2)]

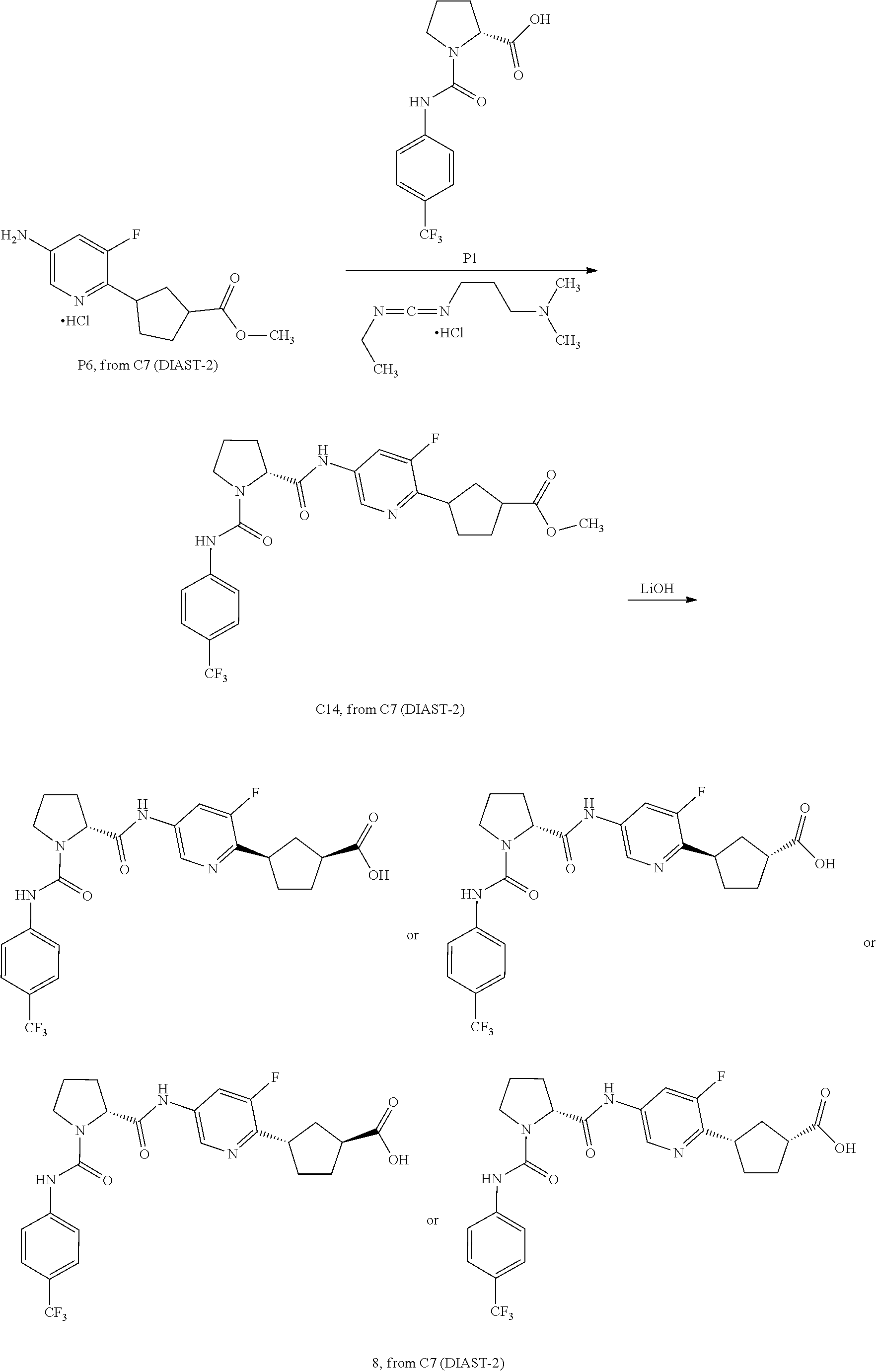

### Step 1. Synthesis of methyl 3-{3-fluoro-5-[(1-{[4-(trifluoromethyl)phenyl]carbamoyl}-D-prolyl)amino]pyridin-2-yl}cyclopentane-1-carboxylate [C₁₋₄, from C7 (DIAST-2)]

1-[3-(Dimethylamino)propyl]-3-ethylcarbodiimide hydrochloride (23.9 mg, 0.125 mmol) was added to a solution of P1 (37.6 mg, 0.124 mmol) and P6, from C7 (DIAST-2) (28.5 mg, 0.104 mmol) in dichloromethane (0.89 mL), whereupon the reaction mixture was stirred at 15° C. for 1 hour. Purification via thin-layer chromatography (Eluant: ethyl acetate) provided C₁₋₄, from C7 (DIAST-2) as a colorless gum. Yield: 40 mg, 77 μmol, 74%. LCMS m/z 523.0 [M+H]⁺.

### Step 2. Synthesis of (1S,3R)-3-{3-fluoro-5-[(1-{[4-(trifluoromethyl)phenyl]carbamoyl}-D-prolyl)amino]pyridin-2-yl}cyclopentane-1-carboxylic acid, (1R,3R)-3-{3-fluoro-5-[(1-{[4-(trifluoromethyl)phenyl]carbamoyl}-D-prolyl)amino]pyridin-2-yl}cyclopentane-1-carboxylic acid, (1S,3S)-3-{3-fluoro-5-[(1-{[4-(trifluoromethyl)phenyl]carbamoyl}-D-prolyl)amino]pyridin-2-yl}cyclopentane-1-carboxylic acid, or (1R,3S)-3-{3-fluoro-5-[(1-{[4-(trifluoromethyl)phenyl]carbamoyl}-D-prolyl)amino]pyridin-2-yl}cyclopentane-1-carboxylic acid [8, from C7 (DIAST-2)]

Lithium hydroxide monohydrate (28.9 mg, 0.689 mmol) was added to a 15° C. solution of C₁₋₄, from C7 (DIAST-2) (40 mg, 77 μmol) in a mixture of tetrahydrofuran (2 mL) and water (0.6 mL). The reaction mixture was stirred at 15° C. for 16 hours, whereupon it was acidified to pH 6 by the addition of hydrochloric acid (1 M; 0.7 mL, 0.7 mmol). Purification via reversed-phase HPLC (Column: WePure Biotech XP tC18, 40×150 mm, 7 μm; Mobile phase A: water containing 0.05% ammonium hydroxide and 10 mM ammonium bicarbonate; Mobile phase B: acetonitrile; Gradient: 13% to 33% B; Flow rate: 60 mL/minute) provided (1S,3R)-3-{3-fluoro-5-[(1-{[4-(trifluoromethyl)phenyl]carbamoyl}-D-prolyl)amino]pyridin-2-yl}cyclopentane-1-carboxylic acid, (1R,3R)-3-{3-fluoro-5-[(1-{[4-(trifluoromethyl)phenyl]carbamoyl}-D-prolyl)amino]pyridin-2-yl}cyclopentane-1-carboxylic acid, (1S,3S)-3-{3-fluoro-5-[(1-{[4-(trifluoromethyl)phenyl]carbamoyl}-D-prolyl)amino]pyridin-2-yl}cyclopentane-1-carboxylic acid, or (1R,3S)-3-{3-fluoro-5-[(1-{[4-(trifluoromethyl)phenyl]carbamoyl}-D-prolyl)amino]pyridin-2-yl}cyclopentane-1-carboxylic acid [8, from C7 (DIAST-2)] as a white solid. Yield: 12.1 mg, 23.8 μmol, 31%. LCMS m/z 509.3 [M+H]⁺. ¹H NMR (400 MHz, CDCl₃), aliphatic integrations are approximate: δ 10.02 (br s, 1H), 8.38-8.28 (m, 1H), 8.00 (d, J=11.6 Hz, 1H), 7.56 (AB quartet, J_{AB}=9.0 Hz, Δv_{AB}=10.9 Hz, 4H), 6.58 (s, 1H), 4.76 (br d, J=8 Hz, 1H), 3.70-3.58 (m, 2H), 3.52-3.42 (m, 1H), 3.17-3.06 (m, 1H), 2.65-2.56 (m, 1H), [2.35-2.07 (m) and 2.06-1.81 (m), total 9H].

### Example 9

### (1S,3R)-3-{3-Fluoro-5-[(1-{[4-(trifluoromethyl)phenyl]carbamoyl}-D-prolyl)amino]pyridin-2-yl}cyclopentane-1-carboxylic acid, (1R,3R)-3-{3-Fluoro-5-[(1-{[4-(trifluoromethyl)phenyl]carbamoyl}-D-prolyl)amino]pyridin-2-yl}cyclopentane-1-carboxylic acid, (1S,3S)-3-{3-Fluoro-5-[(1-{[4-(trifluoromethyl)phenyl]carbamoyl}-D-prolyl)amino]pyridin-2-yl}cyclopentane-1-carboxylic acid, or (1R,3S)-3-{3-Fluoro-5-[(1-{[4-(trifluoromethyl)phenyl]carbamoyl}-D-prolyl)amino]pyridin-2-yl}cyclopentane-1-carboxylic acid [9, from C8 (DIAST-3)]

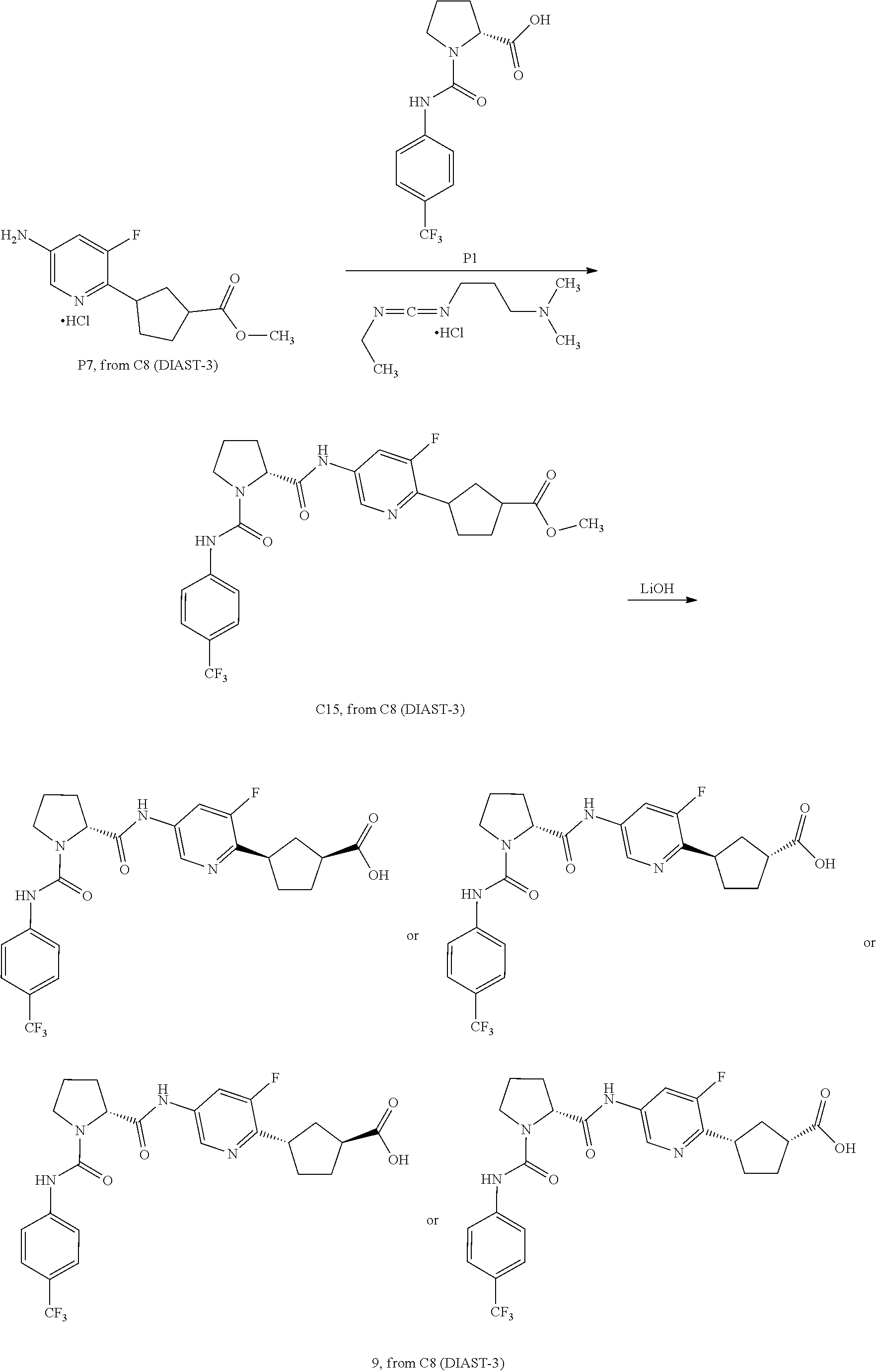

### Step 1. Synthesis of methyl 3-{3-fluoro-5-[(1-{[4-(trifluoromethyl)phenyl]carbamoyl}-D-prolyl)amino]pyridin-2-yl}cyclopentane-1-carboxylate [C₁₅, from C8 (DIAST-3)]

To a 15° C. solution of P1 (34.3 mg, 0.113 mmol) and P7, from C8 (DIAST-3) (26 mg, 95 μmol) in dichloromethane (0.816 mL) was added 1-[3-(dimethylamino)propyl]-3-ethylcarbodiimide hydrochloride (21.8 mg, 0.114 mmol). After the reaction mixture had been stirred at 15° C. for 1 hour, it was subjected to thin-layer chromatography (Eluant: ethyl acetate), affording C₁₅, from C8 (DIAST-3) as a colorless gum. Yield: 42 mg, 80 μmol, 84%. LCMS m/z 523.0 [M+H]⁺.

### Step 2. Synthesis of (1S,3R)-3-{3-fluoro-5-[(1-{[4-(trifluoromethyl)phenyl]carbamoyl}-D-prolyl)amino]pyridin-2-yl}cyclopentane-1-carboxylic acid, (1R,3R)-3-{3-fluoro-5-[(1-{[4-(trifluoromethyl)phenyl]carbamoyl}-D-prolyl)amino]pyridin-2-yl}cyclopentane-1-carboxylic acid, (1S,3S)-3-{3-fluoro-5-[(1-{[4-(trifluoromethyl)phenyl]carbamoyl}-D-prolyl)amino]pyridin-2-yl}cyclopentane-1-carboxylic acid, or (1R,3S)-3-{3-fluoro-5-[(1-{[4-(trifluoromethyl)phenyl]carbamoyl}-D-prolyl)amino]pyridin-2-yl}cyclopentane-1-carboxylic acid [9, from C8 (DIAST-3)]

To a 15° C. solution of C₁₅, from C8 (DIAST-3) (42 mg, 80 μmol) in a mixture of tetrahydrofuran (1.5 mL) and water (0.5 mL) was added lithium hydroxide monohydrate (30.4 mg, 0.724 mmol). The reaction mixture was stirred at 15° C. for 16 hours, whereupon it was acidified to pH 6 by addition of hydrochloric acid (1 M; 0.7 mL, 0.7 mmol). Reversed-phase HPLC (Column: WePure Biotech XP tC18, 40×150 mm, 7 μm; Mobile phase A: water containing 0.05% ammonium hydroxide and 10 mM ammonium bicarbonate; Mobile phase B: acetonitrile; Gradient: 6% to 46% B; Flow rate: 60 mL/minute) afforded (1S,3R)-3-{3-fluoro-5-[(1-{[4-(trifluoromethyl)phenyl]carbamoyl}-D-prolyl)amino]pyridin-2-yl}cyclopentane-1-carboxylic acid, (1R,3R)-3-{3-fluoro-5-[(1-{[4-(trifluoromethyl)phenyl]carbamoyl}-D-prolyl)amino]pyridin-2-yl}cyclopentane-1-carboxylic acid, (1S,3S)-3-{3-fluoro-5-[(1-{[4-(trifluoromethyl)phenyl]carbamoyl}-D-prolyl)amino]pyridin-2-yl}cyclopentane-1-carboxylic acid, or (1R,3S)-3-{3-fluoro-5-[(1-{[4-(trifluoromethyl)phenyl]carbamoyl}-D-prolyl)amino]pyridin-2-yl}cyclopentane-1-carboxylic acid [9, from C8 (DIAST-3)] as a white solid. Yield: 10.7 mg, 21.0 μmol, 26%. LCMS m/z 509.3 [M+H]⁺. ¹H NMR (400 MHz, CDCl₃) δ 10.19 (s, 1H), 8.48 (d, J=2.1 Hz, 1H), 8.23 (dd, J=11.3, 2.1 Hz, 1H), 7.60-7.51 (m, 4H), 6.70 (s, 1H), 4.78 (br d, J=7.9 Hz, 1H), 3.87-3.78 (m, 1H), 3.72-3.64 (m, 1H), 3.55-3.46 (m, 1H), 3.16-3.04 (m, 1H), 2.57-2.47 (m, 1H), 2.42-2.28 (m, 2H), 2.28-1.95 (m, 5H), 1.91-1.77 (m, 2H).

### Example 10

### (1S,3R)-3-{3-Fluoro-5-[(1-{[4-(trifluoromethyl)phenyl]carbamoyl}-D-prolyl)amino]pyridin-2-yl}cyclopentane-1-carboxylic acid, (1R,3R)-3-{3-Fluoro-5-[(1-{[4-(trifluoromethyl)phenyl]carbamoyl}-D-prolyl)amino]pyridin-2-yl}cyclopentane-1-carboxylic acid, (1S,3S)-3-{3-Fluoro-5-[(1-{[4-(trifluoromethyl)phenyl]carbamoyl}-D-prolyl)amino]pyridin-2-yl}cyclopentane-1-carboxylic acid, or (1R,3S)-3-{3-Fluoro-5-[(1-{[4-(trifluoromethyl)phenyl]carbamoyl}-D-prolyl)amino]pyridin-2-yl}cyclopentane-1-carboxylic acid [10, from C9 (DIAST-4)]

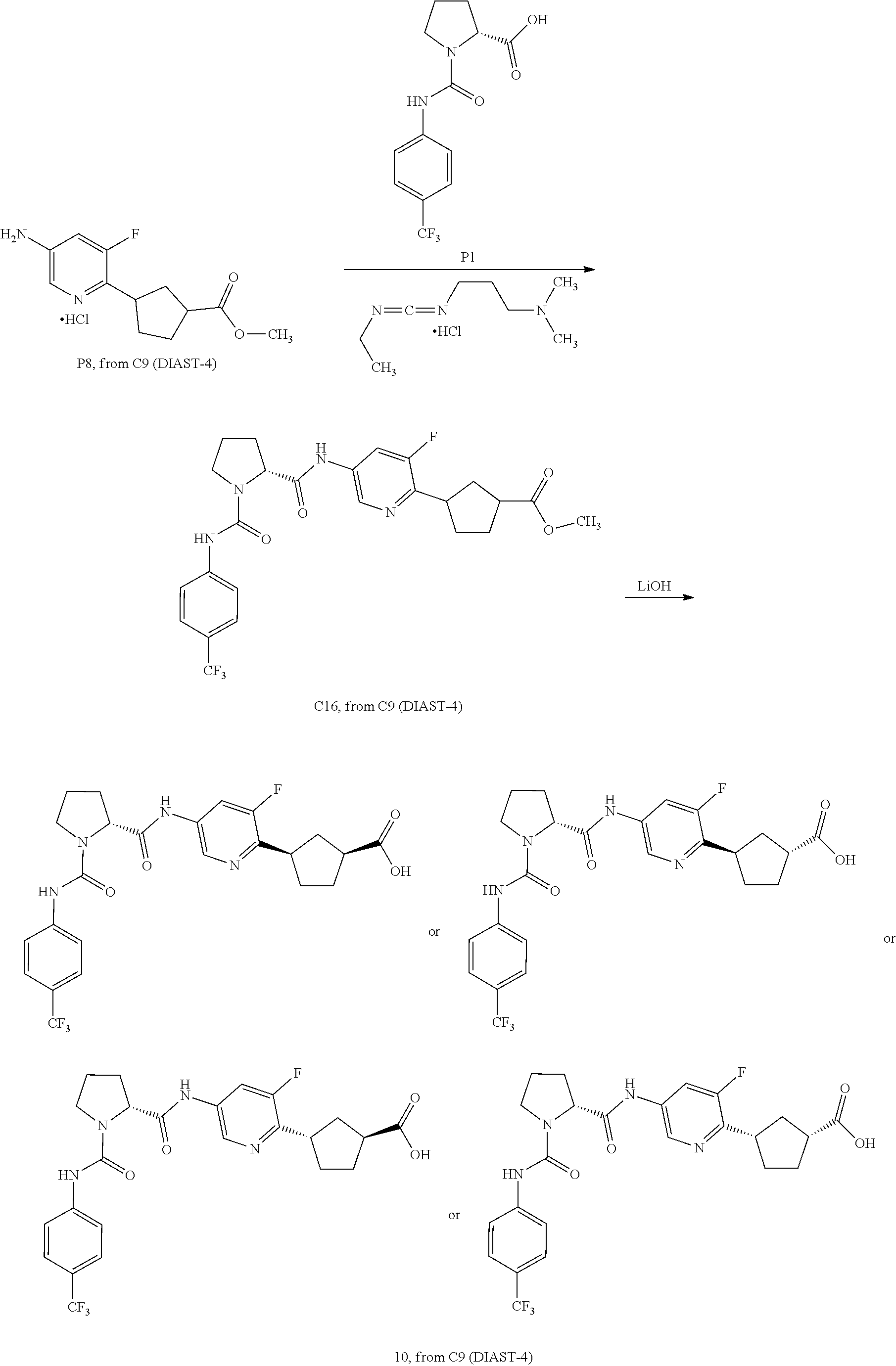

### Step 1. Synthesis of methyl 3-{3-fluoro-5-[(1-{[4-(trifluoromethyl)phenyl]carbamoyl}-D-prolyl)amino]pyridin-2-yl}cyclopentane-1-carboxylate [C16, from C9 (DIAST-4)]

1-[3-(Dimethylamino)propyl]-3-ethylcarbodiimide hydrochloride (28.0 mg, 0.146 mmol) was added to a 15° C. solution of P1 (44.2 mg, 0.146 mmol) and P8, from C9 (DIAST-4) (33.5 mg, 0.122 mmol) in dichloromethane (1.05 mL), whereupon the reaction mixture was stirred at 15° C. for 1 hour. Purification via by thin-layer chromatography (Eluant: ethyl acetate) provided C16, from C9 (DIAST-4) as a colorless gum. Yield: 40 mg, 77 μmol, 63%. LCMS m/z 523.0 [M+H]⁺.

### Step 2. Synthesis of (1S,3R)-3-{3-fluoro-5-[(1-{[4-(trifluoromethyl)phenyl]carbamoyl}-D-prolyl)amino]pyridin-2-yl}cyclopentane-1-carboxylic acid, (1R,3R)-3-{3-fluoro-5-[(1-{[4-(trifluoromethyl)phenyl]carbamoyl}-D-prolyl)amino]pyridin-2-yl}cyclopentane-1-carboxylic acid, (1S,3S)-3-{3-fluoro-5-[(1-{[4-(trifluoromethyl)phenyl]carbamoyl}-D-prolyl)amino]pyridin-2-yl}cyclopentane-1-carboxylic acid, or (1R,3S)-3-{3-fluoro-5-[(1-{[4-(trifluoromethyl)phenyl]carbamoyl}-D-prolyl)amino]pyridin-2-yl}cyclopentane-1-carboxylic acid [10, from C9 (DIAST-4)]

To a 15° C. solution of C16, from C9 (DIAST-4) (40 mg, 77 μmol) in a mixture of tetrahydrofuran (2 mL) and water (0.6 mL) was added lithium hydroxide monohydrate (28.9 mg, 0.689 mmol). After the reaction mixture had been stirred at 15° C. for 16 hours, it was acidified to pH 6 by addition of hydrochloric acid (1 M; 0.7 mL, 0.7 mmol). Reversed-phase HPLC (Column: WePure Biotech XP tC18, 40×150 mm, 7 μm; Mobile phase A: water containing 0.05% ammonium hydroxide and 10 mM ammonium bicarbonate; Mobile phase B: acetonitrile; Gradient: 3% to 43% B; Flow rate: 60 mL/minute) afforded (1S,3R)-3-{3-fluoro-5-[(1-{[4-(trifluoromethyl)phenyl]carbamoyl}-D-prolyl)amino]pyridin-2-yl}cyclopentane-1-carboxylic acid, (1R,3R)-3-{3-fluoro-5-[(1-{[4-(trifluoromethyl)phenyl]carbamoyl}-D-prolyl)amino]pyridin-2-yl}cyclopentane-1-carboxylic acid, (1S,3S)-3-{3-fluoro-5-[(1-{[4-(trifluoromethyl)phenyl]carbamoyl}-D-prolyl)amino]pyridin-2-yl}cyclopentane-1-carboxylic acid, or (1R,3S)-3-{3-fluoro-5-[(1-{[4-(trifluoromethyl)phenyl]carbamoyl}-D-prolyl)amino]pyridin-2-yl}cyclopentane-1-carboxylic acid [10, from C9 (DIAST-4)] as a white solid. Yield: 5.3 mg, 10 μmol, 13%. LCMS m/z 509.3 [M+H]⁺. ¹H NMR (400 MHz, CDCl₃) δ 9.95 (br s, 1H), 8.28 (d, J=2.1 Hz, 1H), 7.96 (dd, J=11.5, 2.1 Hz, 1H), 7.56 (AB quartet, J_{AB}=8.8 Hz, Δv_{AB}=14.2 Hz, 4H), 6.53 (s, 1H), 4.76 (br d, J=7.9 Hz, 1H), 3.67-3.57 (m, 2H), 3.52-3.42 (m, 1H), 3.16-3.05 (m, 1H), 2.67-2.58 (m, 1H), [2.33-2.08 (m) and 2.03-1.82 (m), total 9H].

Each of Compounds 7, 8, 9, and 10, is a different one of the 4 diastereomers and all the 4 diastereomers were prepared in Examples 7, 8, 9, and 10.

### Example 11

### (1S,3R)-3-{3-Fluoro-5-[(1-{[4-(trifluoromethoxy)phenyl]carbamoyl}-D-prolyl)amino]pyridin-2-yl}cyclopentane-1-carboxylic acid, (1R,3R)-3-{3-Fluoro-5-[(1-{[4-(trifluoromethoxy)phenyl]carbamoyl}-D-prolyl)amino]pyridin-2-yl}cyclopentane-1-carboxylic acid, (1S,3S)-3-{3-Fluoro-5-[(1-{[4-(trifluoromethoxy)phenyl]carbamoyl}-D-prolyl)amino]pyridin-2-yl}cyclopentane-1-carboxylic acid, or (1R,3S)-3-{3-Fluoro-5-[(1-{[4-(trifluoromethoxy)phenyl]carbamoyl}-D-prolyl)amino]pyridin-2-yl}cyclopentane-1-carboxylic acid (11)

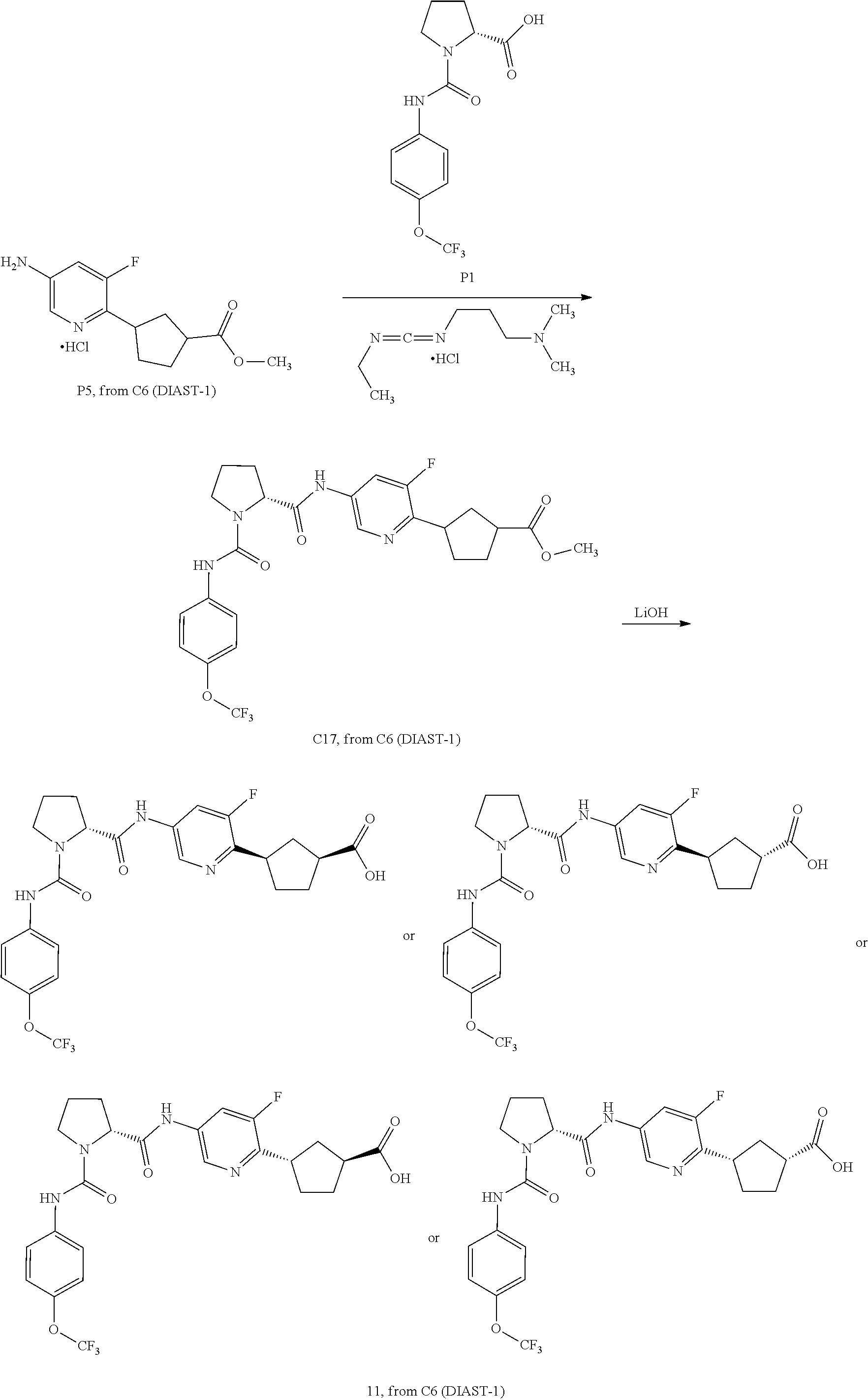

### Step 1. Synthesis of methyl 3-{3-fluoro-5-[(1-{[4-(trifluoromethoxy)phenyl]carbamoyl}-D-prolyl)amino]pyridin-2-yl}cyclopentane-1-carboxylate [C₁₇, from C6 (DIAST-1)]

1-[3-(Dimethylamino)propyl]-3-ethylcarbodiimide hydrochloride (26.7 mg, 0.139 mmol) was added to a 15° C. solution of P2 (40.7 mg, 0.128 mmol) and P5, from C6 (DIAST-1) (32 mg, 0.12 mmol) in dichloromethane (1.0 mL), whereupon the reaction mixture was stirred at 15° C. for 1 hour. Purification via thin-layer chromatography (Eluant: ethyl acetate) provided C₁₇, from C6 (DIAST-1) as a colorless gum. Yield: 40 mg, 74 μmol, 62%. LCMS m/z 539.0 [M+H]⁺.

### Step 2. Synthesis of (1S,3R)-3-{3-fluoro-5-[(1-{[4-(trifluoromethoxy)phenyl]carbamoyl}-D-prolyl)amino]pyridin-2-yl}cyclopentane-1-carboxylic acid, (1R,3R)-3-{3-fluoro-5-[(1-{[4-(trifluoromethoxy)phenyl]carbamoyl}-D-prolyl)amino]pyridin-2-yl}cyclopentane-1-carboxylic acid, (1S,3S)-3-{3-fluoro-5-[(1-{[4-(trifluoromethoxy)phenyl]carbamoyl}-D-prolyl)amino]pyridin-2-yl}cyclopentane-1-carboxylic acid, or (1R,3S)-3-{3-fluoro-5-[(1-{[4-(trifluoromethoxy)phenyl]carbamoyl}-D-prolyl)amino]pyridin-2-yl}cyclopentane-1-carboxylic acid [11, from C6 (DIAST-1)]

To a 15° C. solution of C₁₇, from C6 (DIAST-1) (40 mg, 74 μmol) in a mixture of tetrahydrofuran (2.0 mL) and water (0.6 mL) was added lithium hydroxide monohydrate (28.1 mg, 0.670 mmol). The reaction mixture was stirred at 15° C. for 16 hours, whereupon it was acidified to pH 6 by addition of hydrochloric acid (1 M; 0.7 mL, 0.7 mmol) and subjected to reversed-phase HPLC (Column: WePure Biotech XP tC18, 40×150 mm, 7 μm; Mobile phase A: water containing 0.05% ammonium hydroxide and 10 mM ammonium bicarbonate; Mobile phase B: acetonitrile; Gradient: 6% to 46% B; Flow rate: 60 mL/minute), affording (1S,3R)-3-{3-fluoro-5-[(1-{[4-(trifluoromethoxy)phenyl]carbamoyl}-D-prolyl)amino]pyridin-2-yl}cyclopentane-1-carboxylic acid, (1R,3R)-3-{3-fluoro-5-[(1-{[4-(trifluoromethoxy)phenyl]carbamoyl}-D-prolyl)amino]pyridin-2-yl}cyclopentane-1-carboxylic acid, (1S,3S)-3-{3-fluoro-5-[(1-{[4-(trifluoromethoxy)phenyl]carbamoyl}-D-prolyl)amino]pyridin-2-yl}cyclopentane-1-carboxylic acid, or (1R,3S)-3-{3-fluoro-5-[(1-{[4-(trifluoromethoxy)phenyl]carbamoyl}-D-prolyl)amino]pyridin-2-yl}cyclopentane-1-carboxylic acid [11, from C6 (DIAST-1)] as a white solid. Yield: 16.7 mg, 31.8 μmol, 43%. LCMS m/z 525.3 [M+H]⁺. ¹H NMR (400 MHz, CDCl₃) δ 10.30 (s, 1H), 8.41 (d, J=2.1 Hz, 1H), 8.20 (dd, J=11.3, 2.1 Hz, 1H), 7.44 (d, J=9.0 Hz, 2H), 7.19 (br d, J=8.4 Hz, 2H), 6.52 (s, 1H), 4.76 (br d, J=7.9 Hz, 1H), 3.87-3.78 (m, 1H), 3.67-3.58 (m, 1H), 3.51-3.42 (m, 1H), 3.17-3.05 (m, 1H), 2.62-2.53 (m, 1H), 2.40-2.30 (m, 2H), 2.27-2.10 (m, 3H), 2.08-1.94 (m, 2H), 1.90-1.77 (m, 2H).

### Example 12

### (1S,3R)-3-{3-Fluoro-5-[(1-{[4-(trifluoromethoxy)phenyl]carbamoyl}-D-prolyl)amino]pyridin-2-yl}cyclopentane-1-carboxylic acid, (1R,3R)-3-{3-Fluoro-5-[(1-{[4-(trifluoromethoxy)phenyl]carbamoyl}-D-prolyl)amino]pyridin-2-yl}cyclopentane-1-carboxylic acid, (1S,3S)-3-{3-Fluoro-5-[(1-{[4-(trifluoromethoxy)phenyl]carbamoyl}-D-prolyl)amino]pyridin-2-yl}cyclopentane-1-carboxylic acid, or (1R,3S)-3-{3-Fluoro-5-[(1-{[4-(trifluoromethoxy)phenyl]carbamoyl}-D-prolyl)amino]pyridin-2-yl}cyclopentane-1-carboxylic acid [12, from C7 (DIAST-2)]

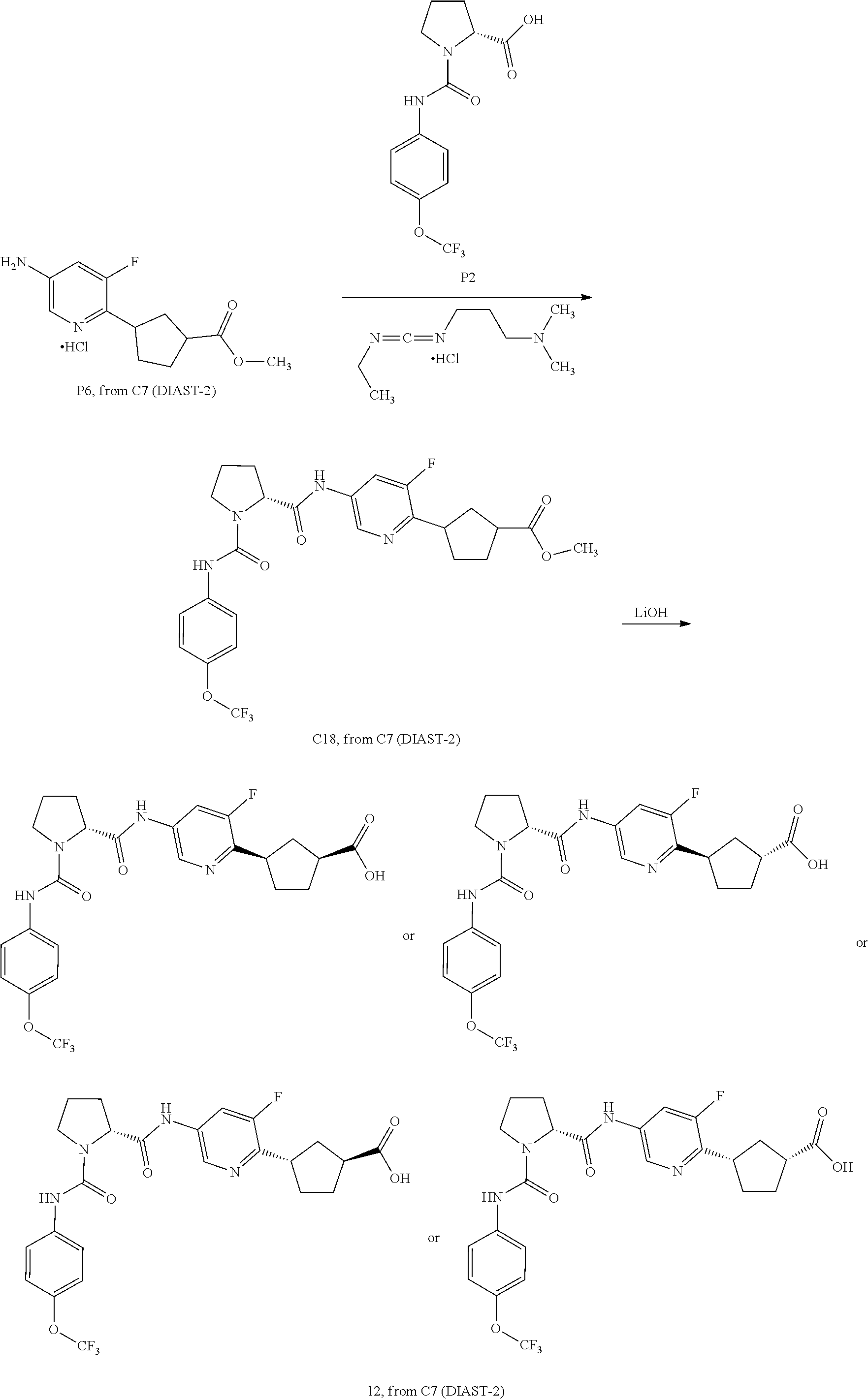

### Step 1. Synthesis of methyl 3-{3-fluoro-5-[(1-{[4-(trifluoromethoxy)phenyl]carbamoyl}-D-prolyl)amino]pyridin-2-yl}cyclopentane-1-carboxylate [C18, from C7 (DIAST-2)]

To a solution of P2 (36.2 mg, 0.114 mmol) and P6, from C7 (DIAST-2) (28.5 mg, 0.104 mmol) in dichloromethane (0.894 mL) was added 1-[3-(dimethylamino)propyl]-3-ethylcarbodiimide hydrochloride (23.8 mg, 0.124 mmol), whereupon the reaction mixture was stirred at 15° C. for 1 hour. Thin-layer chromatography (Eluant: ethyl acetate) subsequently provided C18, from C7 (DIAST-2) as a colorless gum. Yield: 39 mg, 72 μmol, 69%. LCMS m/z 539.0 [M+H]⁺.

### Step 2. Synthesis of (1S,3R)-3-{3-fluoro-5-[(1-{[4-(trifluoromethoxy)phenyl]carbamoyl}-D-prolyl)amino]pyridin-2-yl}cyclopentane-1-carboxylic acid, (1R,3R)-3-{3-fluoro-5-[(1-{[4-(trifluoromethoxy)phenyl]carbamoyl}-D-prolyl)amino]pyridin-2-yl}cyclopentane-1-carboxylic acid, (1S,3S)-3-{3-fluoro-5-[(1-{[4-(trifluoromethoxy)phenyl]carbamoyl}-D-prolyl)amino]pyridin-2-yl}cyclopentane-1-carboxylic acid, or (1R,3S)-3-{3-fluoro-5-[(1-{[4-(trifluoromethoxy)phenyl]carbamoyl}-D-prolyl)amino]pyridin-2-yl}cyclopentane-1-carboxylic acid [12, from C7 (DIAST-2)]

Lithium hydroxide monohydrate (27.4 mg, 0.653 mmol) was added to a 15° C. solution of C18, from C7 (DIAST-2) (39 mg, 72 μmol) in a mixture of tetrahydrofuran (2.0 mL) and water (0.6 mL). After the reaction mixture had been stirred at 15° C. for 16 hours, it was adjusted to pH 6 by addition of hydrochloric acid (1 M, 0.7 mL, 0.7 mmol). Purification via reversed-phase HPLC (Column: WePure Biotech XP tC18, 40×150 mm, 7 μm; Mobile phase A: water containing 0.05% ammonium hydroxide and 10 mM ammonium bicarbonate; Mobile phase B: acetonitrile; Gradient: 13% to 33% B; Flow rate: 60 mL/minute) afforded (1S,3R)-3-{3-fluoro-5-[(1-{[4-(trifluoromethoxy)phenyl]carbamoyl}-D-prolyl)amino]pyridin-2-yl}cyclopentane-1-carboxylic acid, (1R,3R)-3-{3-fluoro-5-[(1-{[4-(trifluoromethoxy)phenyl]carbamoyl}-D-prolyl)amino]pyridin-2-yl}cyclopentane-1-carboxylic acid, (1S,3S)-3-{3-fluoro-5-[(1-{[4-(trifluoromethoxy)phenyl]carbamoyl}-D-prolyl)amino]pyridin-2-yl}cyclopentane-1-carboxylic acid, or (1R,3S)-3-{3-fluoro-5-[(1-{[4-(trifluoromethoxy)phenyl]carbamoyl}-D-prolyl)amino]pyridin-2-yl}cyclopentane-1-carboxylic acid [12, from C7 (DIAST-2)] as a white solid. Yield: 2.1 mg, 4.0 μmol, 6%. LCMS m/z 525.3 [M+H]⁺. ¹H NMR (400 MHz, CDCl₃), aliphatic integrations are approximate: δ 10.11 (br s, 1H), 8.36-8.29 (m, 1H), 8.01 (d, J=11 Hz, 1H), 7.44 (d, J=9.0 Hz, 2H), 7.19 (br d, J=8.8 Hz, 2H), 6.42 (s, 1H), 4.75 (br d, J=7.8 Hz, 1H), 3.71-3.53 (m, 2H), 3.48-3.38 (m, 1H), 3.19-3.05 (m, 1H), 2.70-2.58 (m, 1H), [2.34-2.06 (m) and 2.04-1.81 (m), total 9H].

### Example 13

### (1S,3R)-3-{3-Fluoro-5-[(1-{[4-(trifluoromethoxy)phenyl]carbamoyl}-D-prolyl)amino]pyridin-2-yl}cyclopentane-1-carboxylic acid, (1R,3R)-3-{3-Fluoro-5-[(1-{[4-(trifluoromethoxy)phenyl]carbamoyl}-D-prolyl)amino]pyridin-2-yl}cyclopentane-1-carboxylic acid, (1S,3S)-3-{3-Fluoro-5-[(1-{[4-(trifluoromethoxy)phenyl]carbamoyl}-D-prolyl)amino]pyridin-2-yl}cyclopentane-1-carboxylic acid, or (1R,3S)-3-{3-Fluoro-5-[(1-{[4-(trifluoromethoxy)phenyl]carbamoyl}-D-prolyl)amino]pyridin-2-yl}cyclopentane-1-carboxylic acid [13, from C8 (DIAST-3)]

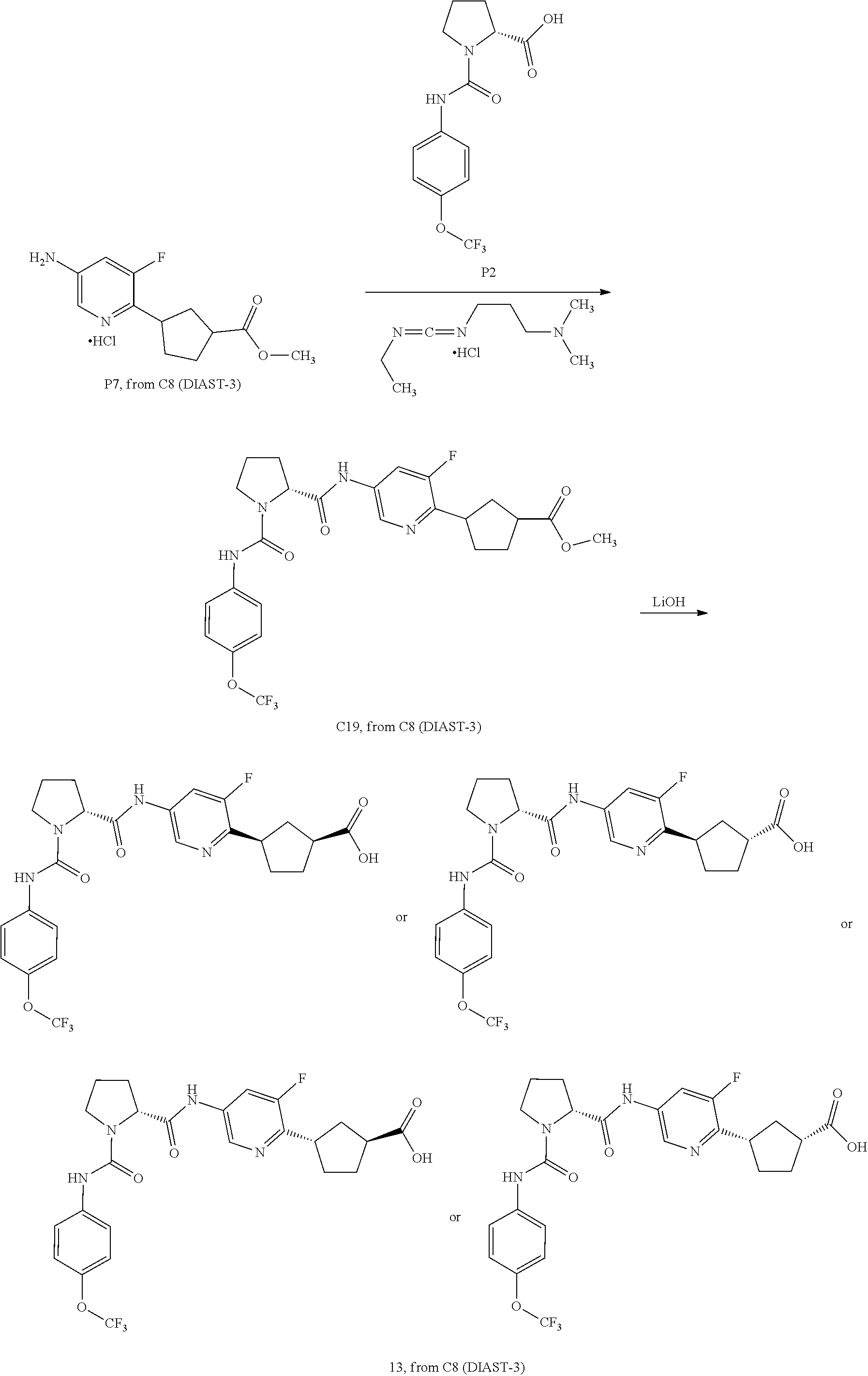

### Step 1. Synthesis of methyl 3-{3-fluoro-5-[(1-{[4-(trifluoromethoxy)phenyl]carbamoyl}-D-prolyl)amino]pyridin-2-yl}cyclopentane-1-carboxylate [C₁₉, from C8 (DIAST-3)]

To a 15° C. solution of P2 (33.0 mg, 0.104 mmol) and P7, from C8 (DIAST-3) (26 mg, 95 μmol) in dichloromethane (0.82 mL) was added 1-[3-(dimethylamino)propyl]-3-ethylcarbodiimide hydrochloride (21.7 mg, 0.113 mmol). After the reaction mixture had been stirred at 15° C. for 1 hour, it was purified via thin-layer chromatography (Eluant: ethyl acetate) to provide C₁₉, from C8 (DIAST-3) as a colorless gum. Yield: 35 mg, 65 μmol, 68%. LCMS m/z 539.0 [M+H]⁺.

### Step 2. Synthesis of (1S,3R)-3-{3-fluoro-5-[(1-{[4-(trifluoromethoxy)phenyl]carbamoyl}-D-prolyl)amino]pyridin-2-yl}cyclopentane-1-carboxylic acid, (1R,3R)-3-{3-fluoro-5-[(1-{[4-(trifluoromethoxy)phenyl]carbamoyl}-D-prolyl)amino]pyridin-2-yl}cyclopentane-1-carboxylic acid, (1S,3S)-3-{3-fluoro-5-[(1-{[4-(trifluoromethoxy)phenyl]carbamoyl}-D-prolyl)amino]pyridin-2-yl}cyclopentane-1-carboxylic acid, or (1R,3S)-3-{3-fluoro-5-[(1-{[4-(trifluoromethoxy)phenyl]carbamoyl}-D-prolyl)amino]pyridin-2-yl}cyclopentane-1-carboxylic acid [13, from C8 (DIAST-3)]

Lithium hydroxide monohydrate (24.5 mg, 0.584 mmol) was added to a 15° C. solution of C₁₉, from C8 (DIAST-3) (35 mg, 65 μmol) in a mixture of tetrahydrofuran (1.5 mL) and water (0.5 mL). The reaction mixture was then stirred at 15° C. for 16 hours, whereupon it was adjusted to pH 6 by addition of hydrochloric acid (1 M; 0.7 mL, 0.7 mmol). Purification via reversed-phase HPLC (Column: WePure Biotech XP tC18, 40×150 mm, 7 μm; Mobile phase A: water containing 0.05% ammonium hydroxide and 10 mM ammonium bicarbonate; Mobile phase B: acetonitrile; Gradient: 7% to 47% B; Flow rate: 60 mL/minute) afforded (1S,3R)-3-{3-fluoro-5-[(1-{[4-(trifluoromethoxy)phenyl]carbamoyl}-D-prolyl)amino]pyridin-2-yl}cyclopentane-1-carboxylic acid, (1R,3R)-3-{3-fluoro-5-[(1-{[4-(trifluoromethoxy)phenyl]carbamoyl}-D-prolyl)amino]pyridin-2-yl}cyclopentane-1-carboxylic acid, (1S,3S)-3-{3-fluoro-5-[(1-{[4-(trifluoromethoxy)phenyl]carbamoyl}-D-prolyl)amino]pyridin-2-yl}cyclopentane-1-carboxylic acid, or (1R,3S)-3-{3-fluoro-5-[(1-{[4-(trifluoromethoxy)phenyl]carbamoyl}-D-prolyl)amino]pyridin-2-yl}cyclopentane-1-carboxylic acid [13, from C8 (DIAST-3)] as a white solid. Yield: 14.7 mg, 28.0 μmol, 43%. LCMS m/z 525.3 [M+H]⁺. ¹H NMR (400 MHz, CDCl₃) δ 10.25 (s, 1H), 8.44 (s, 1H), 8.19 (d, J=11.3 Hz, 1H), 7.44 (d, J=9.0 Hz, 2H), 7.17 (d, J=8.6 Hz, 2H), 6.57 (s, 1H), 4.76 (d, J=7.9 Hz, 1H), 3.86-3.77 (m, 1H), 3.69-3.61 (m, 1H), 3.52-3.43 (m, 1H), 3.17-3.04 (m, 1H), 2.58-2.48 (m, 1H), 2.43-2.27 (m, 2H), 2.27-2.10 (m, 3H), 2.10-1.94 (m, 2H), 1.91-1.77 (m, 2H).

### Example 14

### (1S,3R)-3-{3-Fluoro-5-[(1-{[4-(trifluoromethoxy)phenyl]carbamoyl}-D-prolyl)amino]pyridin-2-yl}cyclopentane-1-carboxylic acid, (1R,3R)-3-{3-Fluoro-5-[(1-{[4-(trifluoromethoxy)phenyl]carbamoyl}-D-prolyl)amino]pyridin-2-yl}cyclopentane-1-carboxylic acid, (1S,3S)-3-{3-Fluoro-5-[(1-{[4-(trifluoromethoxy)phenyl]carbamoyl}-D-prolyl)amino]pyridin-2-yl}cyclopentane-1-carboxylic acid, or (1R,3S)-3-{3-Fluoro-5-[(1-{[4-(trifluoromethoxy)phenyl]carbamoyl}-D-prolyl)amino]pyridin-2-yl}cyclopentane-1-carboxylic acid [14, from C9 (DIAST-4)]

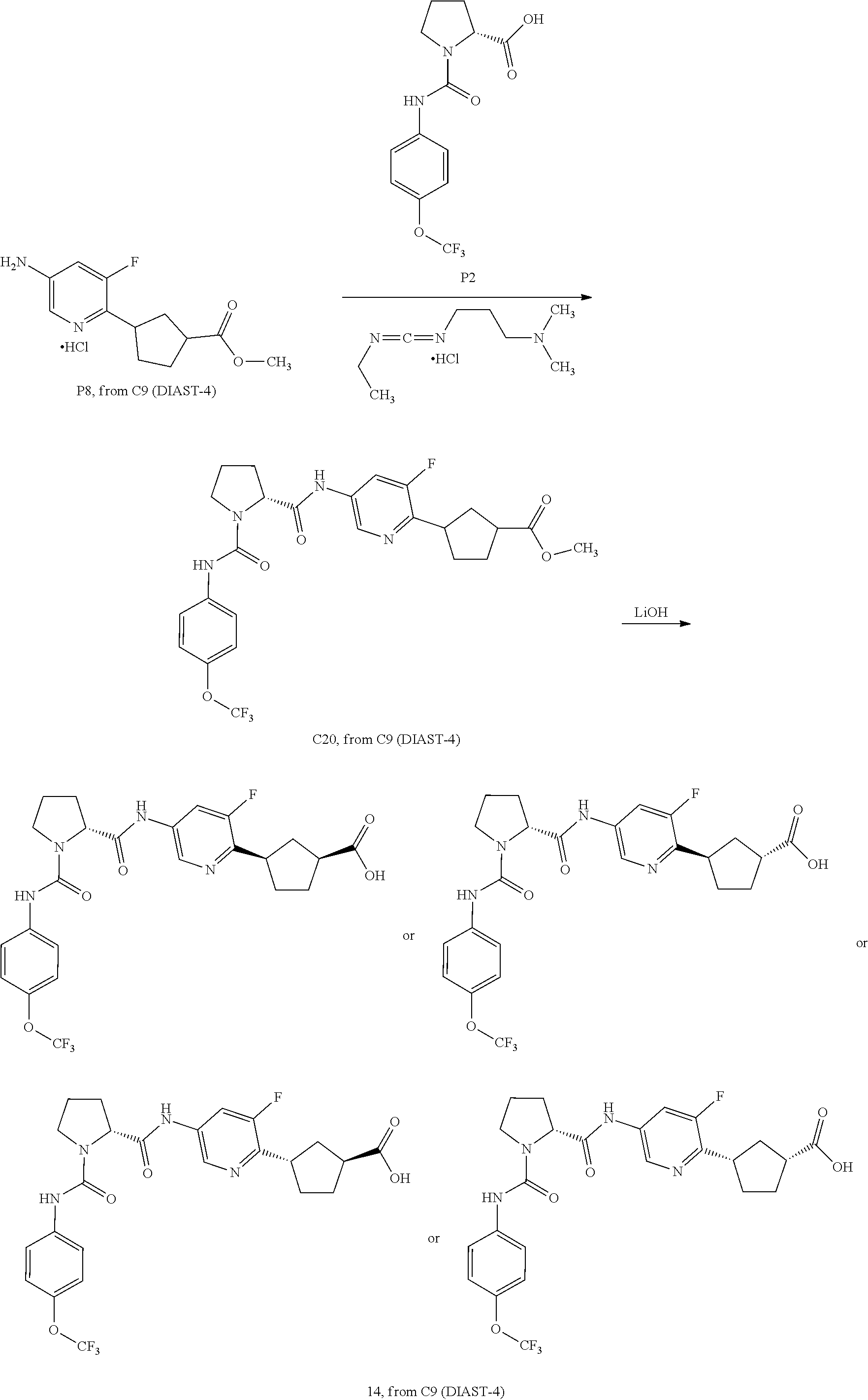

### Step 1. Synthesis of methyl 3-{3-fluoro-5-[(1-{[4-(trifluoromethoxy)phenyl]carbamoyl}-D-prolyl)amino]pyridin-2-yl}cyclopentane-1-carboxylate [C₂₀, from C9 (DIAST-4)]

To a 15° C. solution of P2 (46.5 mg, 0.146 mmol) and P8, from C9 (DIAST-4) (33.5 mg, 0.122 mmol) in dichloromethane (1.0 mL) was added 1-[3-(dimethylamino)propyl]-3-ethylcarbodiimide hydrochloride (28.0 mg, 0.146 mmol), whereupon the reaction mixture was stirred at 15° C. for 1 hour. Purification via thin-layer chromatography (Eluant: ethyl acetate) provided C₂₀, from C9 (DIAST-4) as a colorless gum. Yield: 56 mg, 0.10 mmol, 82%. LCMS m/z 539.0 [M+H]⁺.

### Step 2. Synthesis of (1S,3R)-3-{3-fluoro-5-[(1-{[4-(trifluoromethoxy)phenyl]carbamoyl}-D-prolyl)amino]pyridin-2-yl}cyclopentane-1-carboxylic acid, (1R,3R)-3-{3-fluoro-5-[(1-{[4-(trifluoromethoxy)phenyl]carbamoyl}-D-prolyl)amino]pyridin-2-yl}cyclopentane-1-carboxylic acid, (1S,3S)-3-{3-fluoro-5-[(1-{[4-(trifluoromethoxy)phenyl]carbamoyl}-D-prolyl)amino]pyridin-2-yl}cyclopentane-1-carboxylic acid, or (1R,3S)-3-{3-fluoro-5-[(1-{[4-(trifluoromethoxy)phenyl]carbamoyl}-D-prolyl)amino]pyridin-2-yl}cyclopentane-1-carboxylic acid [14, from C9 (DIAST-4)]

Lithium hydroxide monohydrate (39.3 mg, 0.937 mmol) was added to a 15° C. solution of C₂₀, from C9 (DIAST-4) (56 mg, 0.10 mmol) in a mixture of tetrahydrofuran (2 mL) and water (0.6 mL). The reaction mixture was stirred at 15° C. for 16 hours, whereupon it was acidified to pH 6 by addition of hydrochloric acid (1 M; 0.7 mL, 0.7 mmol). Reversed-phase HPLC purification (Column: WePure Biotech XP tC18, 40×150 mm, 7 μm; Mobile phase A: water containing 0.05% ammonium hydroxide and 10 mM ammonium bicarbonate; Mobile phase B: acetonitrile; Gradient: 3% to 43% B; Flow rate: 60 mL/minute) afforded (1S,3R)-3-{3-fluoro-5-[(1-{[4-(trifluoromethoxy)phenyl]carbamoyl}-D-prolyl)amino]pyridin-2-yl}cyclopentane-1-carboxylic acid, (1R,3R)-3-{3-fluoro-5-[(1-{[4-(trifluoromethoxy)phenyl]carbamoyl}-D-prolyl)amino]pyridin-2-yl}cyclopentane-1-carboxylic acid, (1S,3S)-3-{3-fluoro-5-[(1-{[4-(trifluoromethoxy)phenyl]carbamoyl}-D-prolyl)amino]pyridin-2-yl}cyclopentane-1-carboxylic acid, or (1R,3S)-3-{3-fluoro-5-[(1-{[4-(trifluoromethoxy)phenyl]carbamoyl}-D-prolyl)amino]pyridin-2-yl}cyclopentane-1-carboxylic acid [14, from C9 (DIAST-4)] as a white solid. Yield: 15.3 mg, 29.2 μmol, 29%. LCMS m/z 525.3 [M+H]⁺. ¹H NMR (400 MHz, CDCl₃), aliphatic integrations are approximate: δ 10.03 (br s, 1H), 8.27 (d, J=2 Hz, 1H), 7.96 (br d, J=11.6 Hz, 1H), 7.43 (d, J=9.0 Hz, 2H), 7.19 (br d, J=8.6 Hz, 2H), 6.41 (s, 1H), 4.75 (br d, J=7.9 Hz, 1H), 3.66-3.55 (m, 2H), 3.49-3.39 (m, 1H), 3.15-3.04 (m, 1H), 2.67-2.57 (m, 1H), [2.32-2.06 (m) and 2.03-1.82 (m), total 9H].

Each of Compounds 11, 12, 13, and 14, is a different one of the 4 diastereomers and all the 4 diastereomers were prepared in Examples 11, 12, 13, and 14.

### Example AA. Functional In Vitro GIPR Antagonist Potency Assay

The functional in vitro antagonist potency for test compounds was determined by monitoring intracellular cyclic adenosine monophosphate (cAMP) levels in Chinese hamster ovary (CHO)—K1 cells stably expressing the human Glucose-dependent Insulinotropic Polypeptide Receptor (hGIPR). Following agonist activation, hGIPR associates with the G-protein complex causing the Gas subunit to exchange bound guanosine diphosphate (GDP) for guanosine triphosphate (GTP), followed by dissociation of the Gas-GTP complex. The activated Gas subunit can couple to downstream effectors to regulate the levels of second messengers or cAMP within the cell. Thereby, determination of intracellular cAMP levels allows for pharmacological characterization. Intracellular cAMP levels are quantitated using a homogenous assay utilizing the Homogeneous Time Resolved Fluorescence (HTRF) technology from Perkin Elmer. The method is a competitive immunoassay between native cAMP produced by the cells and cAMP labelled with the acceptor dye, d2. The two entities compete for binding to a monoclonal anti-cAMP antibody labeled with cryptate. The specific signal is inversely proportional to the concentration of cAMP in the cells.

Test compounds were solubilized to a concentration of 30 mM in 100% dimethyl sulfoxide (DMSO). An 11-point dilution series using 1 in 3.162-fold serial dilutions was created in 100% DMSO with a top concentration of 8 mM. The serially diluted compound was spotted with an Echo Acoustic liquid handler (Beckman Coulter) into a 384-well assay plate (Corning, Cat No. 3824) at 50 nL/well with duplicate points at each concentration, at a 200× final assay concentration (FAC). The final compound concentration range in the assay was 40 μM to 400 μM, with a final DMSO concentration of 0.5%.

Frozen assay-ready vials (at 1×10⁷ cells/vial) of CHO-K1 cells stably expressing the Gs-coupled human GIPR receptor (Eurofins, DiscoverX, Cat No. 95-0146C₂) were thawed, counted, and resuspended in assay buffer consisting of Hank's Balanced Salt Solution (HBSS, Lonza Cat No. 10-527) containing 20 mM (4-(2-hydroxyethyl)-1-piperazineethanesulfonic acid (HEPES, Lonza, Cat No. 17-737E), 0.1% bovine serum albumin (BSA, Sigma, Cat No. A7979), and 200 μM 3-isobutyl-1-methylxanthine (IBMX, Sigma, Cat No. 15879) at a density of 4×10⁵ cells/mL. Cells were added to assay plate (5 μL/well of 4×10⁵ cells/mL stock for 2,000 cells/well final) containing 50 nL of 200×FAC test compound, and incubated at 37° C. (95% O₂: 5% CO₂) for 2 hours, with micro-clime lids (Labcyte, Cat No. LLS-0310). Following the 2-hour cell and compound incubation, a stimulation mix comprised of hGIPR agonist human glucose-dependent insulinotropic polypeptide (hGIP, full length, Sigma Cat No. G2269) in assay buffer/0.1% DMSO was added to the assay plate (5 μL/well) at an estimated EC₈₀ FAC (based on previous hGIP agonist curves) and incubated for another 30 minutes with micro-clime lids at 37° C. (95% O₂: 5% CO₂), after which intracellular cAMP levels were quantified as per Perkin Elmer's protocol (5 μL of d2 and then 5 μL cryptate, incubated for 1 hour at room temperature). Emission spectra of samples were measured on a Pherastar plate reader (BMG Labtech Inc) using a HTRF protocol (excitation, 320 nm; emission, 665 nm/620 nm).

hGIP EC₅₀ was determined daily by incubating cells (5 μL/well of 4×10⁵ cells/mL stock, for 2,000 cells/well final) with 50 nL 100% DMSO for 2 hours at 37° C. (95% O₂: 5% CO₂), with a micro-clime lid. Following the 2-hour cell and DMSO incubation, a hGIP concentration response curve at 2×FAC (12-point curve using 1 in 3 serial dilutions, with triplicate points at each concentration, 100 nM final top concentration) in assay buffer/1% DMSO was added (5 μL/well) and incubated for a further 30 minutes with a micro-clime lid at 37° C. (95% O₂: 5% CO₂), after which intracellular cAMP levels were quantified and samples measured as described previously. Experiments passed quality control if the agonist concentration used for stimulation fell between the on-the-day EC₅o-EC₉₀.

Data were analyzed using the ratio of fluorescence intensity at 620 and 665 nm for each well, extrapolated from the cAMP standard curve to express data as nanomolar (nM) cAMP for each well. Data expressed as nM cAMP were then normalized to control wells using ActivityBase (IDBS data management software). Zero percent effect (ZPE) was defined as nM cAMP generated from the hGIP stimulation mix, while 100% effect, or one hundred percent effect (HPE), was defined as nM cAMP generated from the combined effects of hGIP simulation mix+antagonism by 80 μM of (−)-3-(6-(2-methyl-1-(4′-(trifluoromethyl)biphenyl-4-yl)propylamino)nicotinamido)propanoic acid as GIPR antagonist. The concentration and % effect values for each compound were plotted by ActivityBase using a four-parameter logistic dose response equation, and the concentration required for 50% inhibition (IC₅₀) was determined.

Table 2 lists biological activities (IC₅₀) and compound names for Examples 1-14.

TABLE 2
Biological activity and Compound name for Examples 1- 14.

| Example | hGIPR antagonist | hGIPR antagonist |
|---|---|---|
| Number | IC₅₀ (nM)¹ | IC₅₀ replicate count |
| 1 | 66 | 3 |
| 2 | 6.5 | 5 |
| 3 | 56 | 3 |
| 4 | 5.1 | 5 |
| 5 | 97 | 3 |
| 6 | 140 | 4 |
| 7 | 37 | 3 |
| 8 | 87 | 4 |
| 9 | 160 | 4 |
| 10 | 160 | 4 |
| 11 | 9.2 | 4 |
| 12 | 24 | 4 |
| 13 | 17 | 3 |
| 14 | 26 | 4 |

¹Values represent the geometric mean

Throughout this application, various publications are referenced. The disclosures of these publications in their entireties are hereby incorporated by reference into this application for all purposes.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A compound of Formula I: 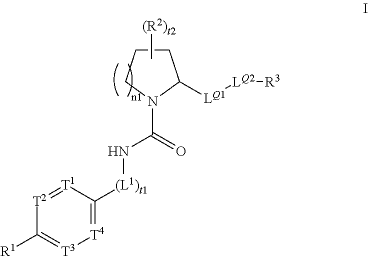
or a pharmaceutically acceptable salt thereof, wherein:
R¹ is H, halogen, —CN, —OR^{1C}, C₁₋₈ alkyl, C₁₋₈ haloalkyl, C₂₋₃ alkenyl, —C₁₋₄ alkyl-(C₃₋₆ cycloalkyl), or C₃₋₆ cycloalkyl, wherein each of the C₁₋₈ alkyl, C₂₋₃ alkenyl, —C₁₋₄ alkyl-(C₃₋₆ cycloalkyl), or C₃₋₆ cycloalkyl is optionally substituted with 1, 2, 3, 4, 5, or 6 substituents each independently selected from halogen, —OH, —CN, C₁₋₄ alkyl, C₁₋₄ haloalkyl, C₁₋₄ alkoxy, and C₁₋₄ haloalkoxy;
R^{1C} is C₁₋₆ alkyl, C₁₋₆ haloalkyl, C₃₋₆ cycloalkyl, or —C₁₋₂ alkyl-(C₃₋₆ cycloalkyl), wherein each of the C₃₋₆ cycloalkyl and —C₁₋₂ alkyl-(C₃₋₆ cycloalkyl) is optionally substituted with 1, 2, 3, 4, 5, or 6 substituents each independently selected from halogen, —OH, —CN, C₁₋₄ alkyl, C₁₋₄ haloalkyl, C₁₋₄ alkoxy, and C₁₋₄ haloalkoxy;
each of T¹, T², T³, and T⁴ is independently CR⁴ or N, provided that only 0, 1, or 2 of T¹, T², T³, and T⁴ is N;
each R⁴ is independently H, halogen, —CN, C₃₋₆ cycloalkyl, —C₁₋₂ alkyl-(C₃₋₆ cycloalkyl), C₁₋₄ alkyl, C₁₋₄ cyanoalkyl, C₁₋₄ haloalkyl, C₁₋₄ alkoxy, or C₁₋₄ haloalkoxy;
or R¹ and an adjacent R⁴, together with the two ring carbon atoms to which they are attached, optionally form a fused 4- or 6-membered cycloalkyl ring, a fused 4- or 6-membered heterocycloalkyl ring, a fused 5- or 6-membered heteroaryl ring, or a fused 6-membered aryl ring, wherein each of the fused rings is optionally substituted with 1, 2, 3, 4, 5, or 6 substituents each independently selected from halogen, —OH, —CN, C₁₋₄ alkyl, C₁₋₄ haloalkyl, C₁₋₄ alkoxy, and C₁₋₄ haloalkoxy;
t1, L¹, and L² are
(a) t1 is 0 or 1, L¹ is C(R^{L1})₂ or [C(R^{L1})₂]₂, and L² is NR^{N}; or
(b) t1 is 0 or 1, L¹ is C(R^{L1})₂, O, or NR^{N}, and L² is C(R^{L2})₂; or
(c) t1 is 1, and -L¹-L²- is —C(R^{L1})₂—O—C(R^{L2})₂—, [C(R^{L1})₂]₃, or —C(R^{L1})₂—N(R^{N})_C(R^{L2})₂—, or -L¹-L²- together form a divalent C₃₋₆ cycloalkyl ring optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy;
each of R^{L1} and R^{L2} is independently H, C₁₋₂ alkyl, C₁₋₂ haloalkyl, C₁₋₂ alkoxy, or C₁₋₂ haloalkoxy;
or two R^{L1}, together with the carbon atom to which they are attached, optionally form C₃₋₆ cycloalkyl or a 3- to 6-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
or two R^{L2}, together with the carbon atom to which they are attached, optionally form C₃₋₆ cycloalkyl or a 3- to 6-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
or —C(R^{L1})₂—C(R^{L2})₂— together optionally form C₃₋₆ cycloalkyl or a 4- to 6-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
R^{N} is H, C₁₋₆ alkyl, C₃₋₆ cycloalkyl, —C₁₋₄ alkyl-(C₃₋₆ cycloalkyl);
n1 and A¹ are
(i) n1 is 1 and A¹ is CH₂; or
(ii) n1 is 2 and A¹ is CH₂, O, S, or NH;
each R² is independently halogen, —OH, C₁₋₄ alkyl, C₁₋₄ haloalkyl, C₁₋₄ alkoxy, C₁₋₄ haloalkoxy, C₃₋₄ cycloalkyl, or —C₁₋₄ alkyl-(C₃₋₆ cycloalkyl), wherein each of the C₁₋₄ alkyl, C₁₋₄ haloalkyl, C₁₋₄ alkoxy, C₁₋₄ haloalkoxy, C₃₋₄ cycloalkyl, or —C₁₋₄ alkyl-(C₃₋₆ cycloalkyl) is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy, provided that when a R² is attached to a ring-forming nitrogen atom of the ring having the variable n1, then the R² is not halogen, —OH, an optionally substituted C₁₋₄ alkoxy, or an optionally substituted C₁₋₄ haloalkoxy;
or two R², when attached to a same ring-forming carbon atom of the ring having the variable n1, together with the ring carbon atom to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
or two R², when attached to two adjacent ring-forming atoms of the ring having the variable n1, together with the two ring-forming atoms to which they are attached, optionally form a fused C₃₋₆ cycloalkyl or a fused 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
t2 is 0, 1, 2, 3, or 4;
L^{Q1} and L^{Q2} are:
(x) L^{Q1} is C(R^{Q1})₂, and L^{Q2} is O; or
(y) L^{Q1} is C(═O), and L^{Q2} is NH;
each R^{Q1} is independently H, C₁₋₂ alkyl, C₁₋₂ haloalkyl, C₃₋₄ cycloalkyl, or —C₁₋₂ alkyl-(C₃₋₄ cycloalkyl);
or two R^{Q1}, together with the carbon atom to which they are attached, optionally form C₃₋₆ cycloalkyl, which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
R³ is R^{3a}, R^{3b}, R^{3c}, R^{3d}, or R^{3e}: 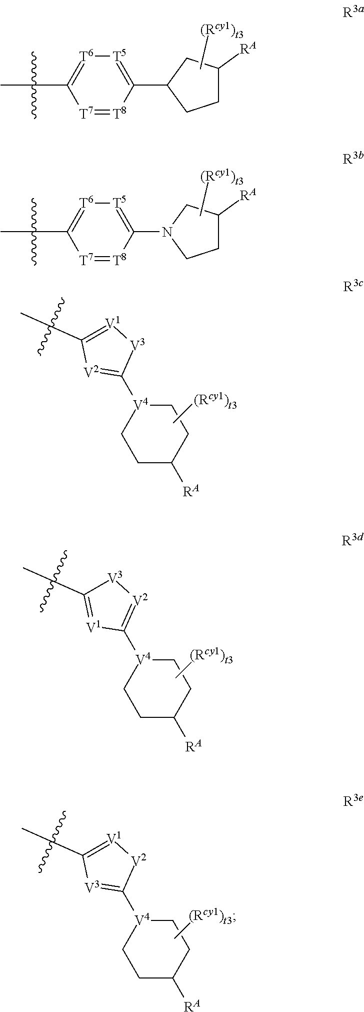
each R^{cy1} is independently halogen, —OH, C₁₋₄ alkyl, C₁₋₄ haloalkyl, C₁₋₄ hydroxylalkyl, C₁₋₄ alkoxy, C₁₋₄ haloalkoxy, C₃₋₄ cycloalkyl, or —C₁₋₄ alkyl-(C₃₋₄ cycloalkyl);
or two R^{cy1}, together with a same ring carbon atom of the cyclohexyl ring to which they are attached, form C₃₋₆ cycloalkyl that is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or —C₁₋₄ alkyl-(C₃₋₄ cycloalkyl);
or two R^{cy1}, each of which is attached to a different ring carbon atom, are linked together form a single bond or a moiety of —CH₂—, —CH₂CH₂—, —CH₂CH₂CH₂—, or —CH₂CH₂CH₂—, which moiety is optionally substituted with 1, 2, 3, or 4 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or —C₁₋₄ alkyl-(C₃₋₄ cycloalkyl);
each of V¹ and V² is independently N or CR^{S1};
V³ is O or S;
V⁴ is CH or N;
each R^{S1} is independently H, C₁₋₄ alkyl, C₁₋₄ haloalkyl, C₃₋₄ cycloalkyl, —C₁₋₄ alkyl-(C₃₋₄ cycloalkyl), C₁₋₄ alkoxy, or C₁₋₄ haloalkoxy;
t3 is 0, 1, 2, or 3;
each of T⁵, T⁶, T⁷, and T³ is independently CR⁵ or N, provided that only 0, 1, or 2 of T⁵, T⁶, T⁷, and T³ can be N;
each R⁵ is independently H, halogen, —CN, C₃₋₆ cycloalkyl, (C₃₋₆ cycloalkyl)—C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
R^{A} is —C(═O)—OH, —C(R^{L3})₂—C(═O)—OH, —C(R^{L3})₂—C(R^{L4})₂—C(═O)—OH, —[C(R^{L3})₂]₃—C(═O)—OH, —O—C(R^{L3})₂—C(═O)—OH, —O—C(R^{L3})₂—C(R^{L4})₂—C(═O)—OH, —O—[C(R^{L3})₂]₃—C(═O)—OH, OH, —C(═O)NH—C(R^{L3})₂—C(═O)—OH, —C(═O)NH—C(R^{L3})₂—C(R^{L4})₂—C(═O)—OH, —C(═O)NH—[C(R^{L3})₂]₃—C(═O)—OH, —C(═O)—N(R^{am1})(R^{am2}), —C(═O)—OR^{es1}, 1H-tetrazol-5-yl, 3-hydroxyisoxazol-5-yl, 5(4H)-oxo-1,2,4-oxadiazol-3-yl-, 5(4H)-oxo-1,2,4-thiadiazol-3-yl-, 2-thioxo-1,3,4-oxadiazol-5-yl-, 4H-1,2,4-triazol-3-yl-, 4-hydroxy-1,2,5-oxadiazol-3-yl, 1-hydroxypyrazol-5-yl, 3-hydroxy-1H-pyrazol-1-yl-, a carboxylic acid bioisostere group, —S(═O)₂NHCF₃, or —C(═O)—NH—S(═O)₂—R¹⁰⁰ wherein R¹⁰⁰ is C₁₋₆ alkyl or phenyl and where the phenyl is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
each of R^{L3} and R^{L4} is independently H, C₁₋₂ alkyl, C₁₋₂ haloalkyl, C₁₋₂ alkoxy, or C₁₋₂ haloalkoxy;
or two R^{L3}, together with the carbon atom to which they are attached, optionally form C₃₋₆ cycloalkyl or a 3- to 6-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
or two R^{L4}, together with the carbon atom to which they are attached, optionally form C₃₋₆ cycloalkyl or a 3- to 6-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
or —C(R^{L3})₂—C(R^{L4})₂— together optionally forms a divalent C₃₋₆ cycloalkyl or a divalent 4- to 6-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
each of R^{am1}and R^{am2} is independently H, C₁₋₆ alkyl, C₃₋₆ cycloalkyl, —C₁₋₄ alkyl-(C₃₋₆ cycloalkyl), phenyl, or —C₁₋₄ alkyl-phenyl, wherein each of the C₁₋₆ alkyl, C₃₋₆ cycloalkyl, C₁₋₄ alkyl-(C₃₋₆ cycloalkyl), phenyl, or —C₁₋₄ alkyl-phenyl is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or —C₁₋₄ alkyl-(C₃₋₄ cycloalkyl);
or R^{am1} and R^{am2} together with the nitrogen atom to which they are attached form a 4- to 8-membered heterocycloalkyl optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or —C₁₋₄ alkyl-(C₃₋₄ cycloalkyl), wherein each of the C₁₋₄ alkyl, C₁₋₄ haloalkyl, C₁₋₄ alkoxy, C₁₋₄ haloalkoxy, C₃₋₄ cycloalkyl, or —C₁₋₄ alkyl-(C₃₋₄ cycloalkyl) is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy; and
R^{es1} is C₁₋₆ alkyl, C₃₋₆ cycloalkyl, —C₁₋₄ alkyl-(C₃₋₆ cycloalkyl), phenyl, or —C₁₋₄ alkyl-phenyl, each of which is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or —C₁₋₄ alkyl-(C₃₋₄ cycloalkyl).

2. The compound of claim 1 wherein the compound of Formula I is a Formula Ia, II, IIa, III, IIIa, IV, IVa, V, Va, VI, VIa, VII, VIIa, VIII, VIIIa, IX, IXa, X, Xa, XI, or XIa: 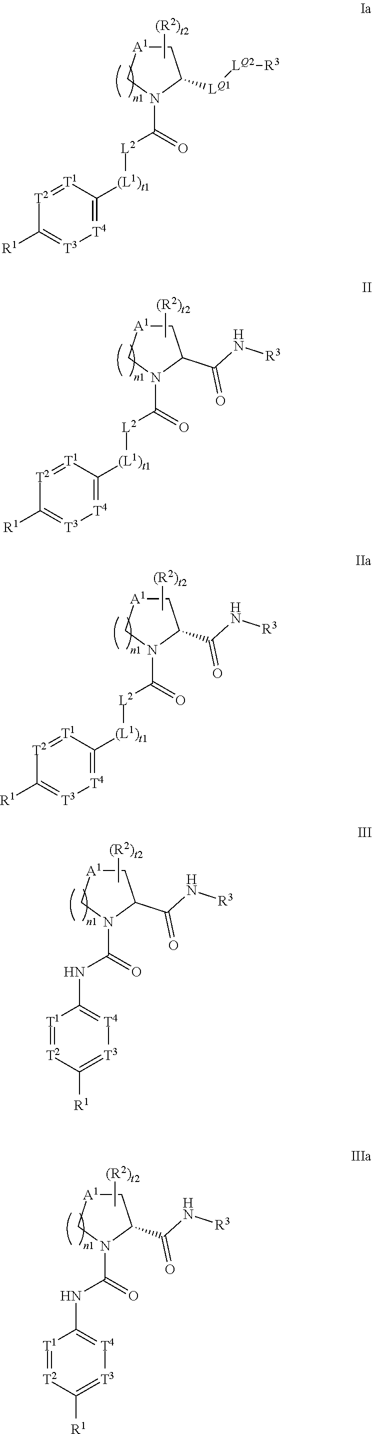 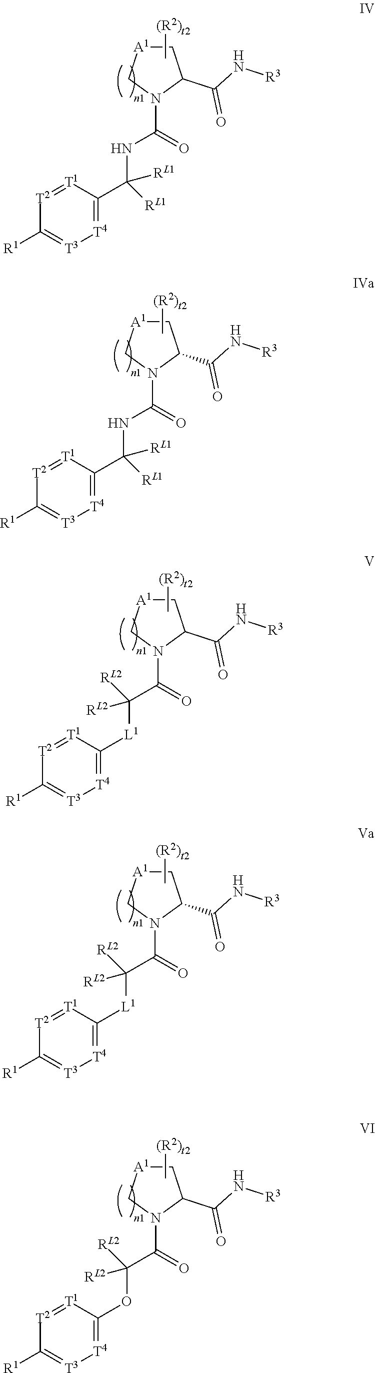 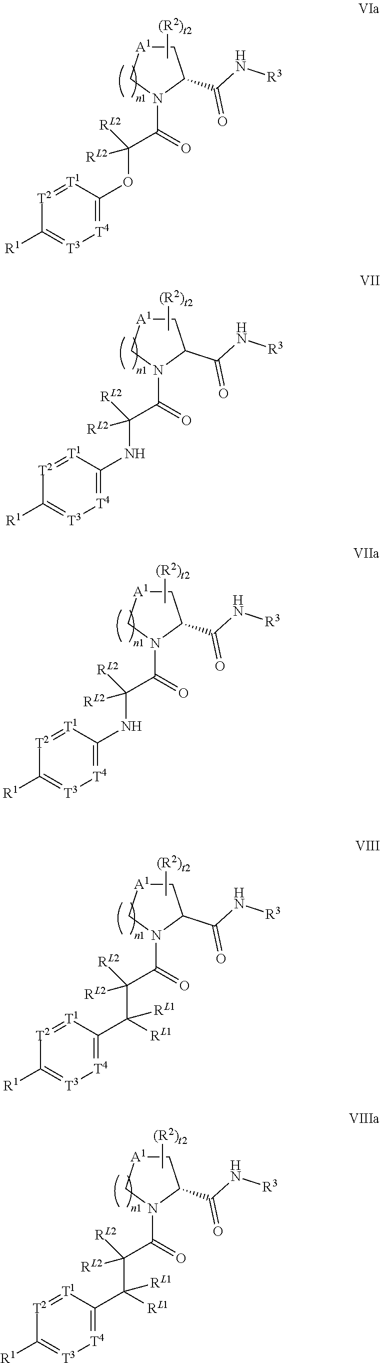 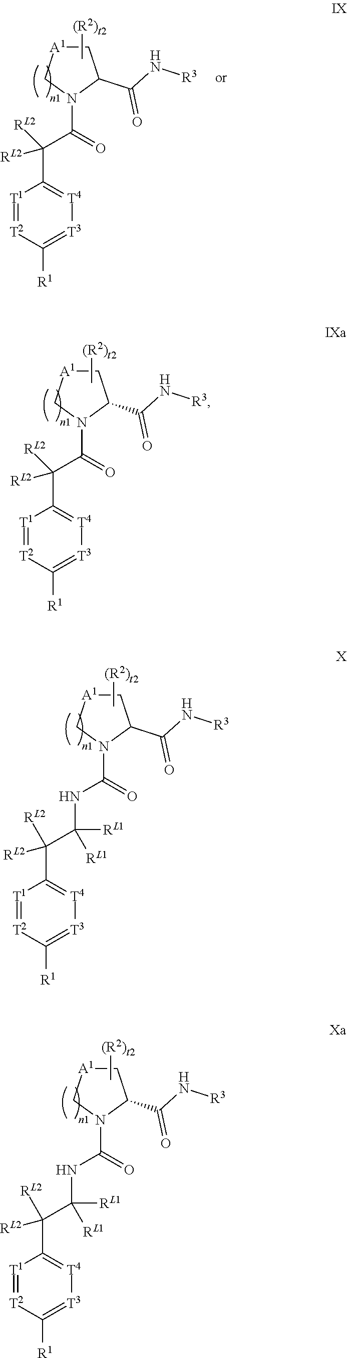 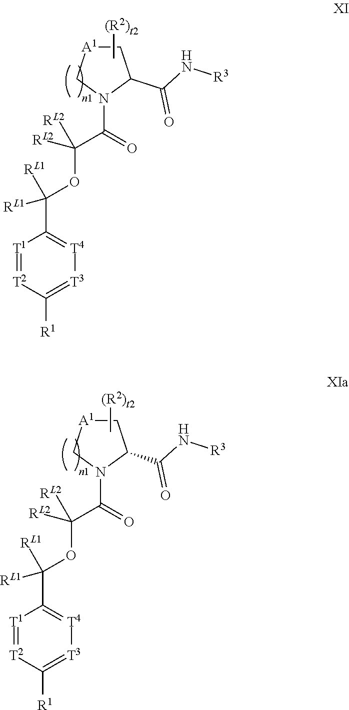
or a pharmaceutically acceptable salt thereof.

3. The compound of claim 1 or a pharmaceutically acceptable salt thereof, wherein R¹ is cyclopropyl, cyclobutyl, cyclopentyl, R^{1a}, R^{1b}, or R^{1c}, 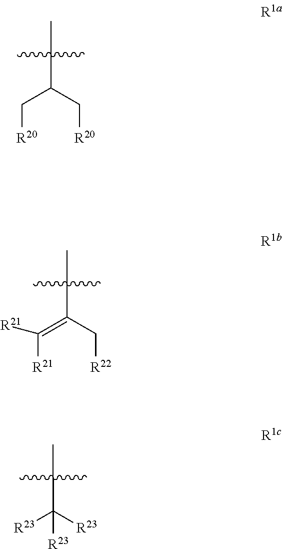
wherein each of the cyclopropyl or cyclobutyl is optionally substituted with 1, 2, 3, or 4 R^{S};
each R²⁰ is independently H, halogen, —OH, C₁₋₂ alkyl, C₁₋₂ haloalkyl, C₁₋₂ alkoxy, or C₁₋₂ haloalkoxy;
each R²¹ is independently H, C₁₋₂ alkyl, or C₁₋₂ haloalkyl;
R²² is H, halogen, C₁₋₂ alkyl, C₁₋₂ hydroxylalkyl, C₁₋₂ haloalkyl, C₁₋₂ alkoxy, or C₁₋₂ haloalkoxy;
each R²³ is independently halogen, C₁₋₂ alkyl, C₁₋₂ hydroxylalkyl, C₁₋₂ haloalkyl, C₁₋₂ alkoxy, or C₁₋₂ haloalkoxy; and
each R^{S} is independently halogen, —OH, C₁₋₂ alkyl, C₁₋₂ hydroxylalkyl, C₁₋₂ haloalkyl, C₁₋₂ alkoxy, or C₁₋₂ haloalkoxy.

4. The compound of claim 1 or a pharmaceutically acceptable salt thereof, wherein R¹ is C₁₋₄ haloalkyl.

5. The compound of claim 1 or a pharmaceutically acceptable salt thereof, wherein each of T¹, T², T³, and T⁴ is independently CR⁴.

6. The compound of claim 1 or a pharmaceutically acceptable salt thereof, wherein one of T¹, T², T³, and T⁴ is N, and the other three are each independently CR⁴.

7. The compound of claim 1 or a pharmaceutically acceptable salt thereof, wherein each R⁴ is independently H, halogen, or C₁₋₂ alkyl.

8. The compound of claim 1 or a pharmaceutically acceptable salt thereof, wherein n1 is 1 and A¹ is CH₂.

9. The compound of claim 1 or a pharmaceutically acceptable salt thereof, wherein each R² is independently halogen, —OH, C₁₋₄ alkyl, C₁₋₄ hydroxylalkyl, C₁₋₄ haloalkyl, C₁₋₄ alkoxy, C₁₋₄ haloalkoxy, C₃₋₄ cycloalkyl, or —C₁₋₄ alkyl-(C₃₋₄ cycloalkyl); and t2 is 0, 1, or 2.

10. The compound of claim 1 or a pharmaceutically acceptable salt thereof, wherein t2 is 0.

11. The compound of claim 1 or a pharmaceutically acceptable salt thereof, wherein R³ is R^{3c}.

12. The compound of claim 1 or a pharmaceutically acceptable salt thereof, wherein V¹ is CR^{S1}; V² is N; and V³ is S.

13. The compound of claim 12 or a pharmaceutically acceptable salt thereof, wherein R^{S1} is H or C₁₋₄ alkyl.

14. The compound of claim 13 or a pharmaceutically acceptable salt thereof, wherein R^{S1} is H.

15. The compound of claim 1 or a pharmaceutically acceptable salt thereof, wherein V⁴ is CH.

16. The compound of claim 11 or a pharmaceutically acceptable salt thereof, wherein each R^{cy1} is independently halogen, —OH, C₁₋₄ alkyl, C₁₋₄ haloalkyl, C₁₋₄ hydroxylalkyl, C₁₋₄ alkoxy, C₁₋₄ haloalkoxy, C₃₋₄ cycloalkyl, or —C₁₋₄ alkyl-(C₃₋₄ cycloalkyl); and t3 is 0 or 1.

17. The compound of claim 11 or a pharmaceutically acceptable salt thereof, wherein t3 is 0.

18. The compound of claim 1 or a pharmaceutically acceptable salt thereof, wherein R^{A} is —C(═O)—OH.

19. A compound selected from the group consisting of:
(1S,4s)-4-{4-[(1-{[4-(trifluoromethyl)phenyl]carbamoyl}-D-prolyl)amino]-1,3-thiazol-2-yl}cyclohexane-1-carboxylic acid;
(1R,4r)-4-{4-[(1-{[4-(trifluoromethyl)phenyl]carbamoyl}-D-prolyl)amino]-1,3-thiazol-2-yl}cyclohexane-1-carboxylic acid;
(1S,4s)-4-{4-[(1-{[4-(trifluoromethoxy)phenyl]carbamoyl}-D-prolyl)amino]-1,3-thiazol-2-yl}cyclohexane-1-carboxylic acid;
(1R,4r)-4-{4-[(1-{[4-(trifluoromethoxy)phenyl]carbamoyl}-D-prolyl)amino]-1,3-thiazol-2-yl}cyclohexane-1-carboxylic acid;
(3S)-1-{3-fluoro-5-[(1-{[4-(trifluoromethoxy)phenyl]carbamoyl}-D-prolyl)amino]pyridin-2-yl}pyrrolidine-3-carboxylic acid;
(3R)-1-{3-fluoro-5-[(1-{[4-(trifluoromethoxy)phenyl]carbamoyl}-D-prolyl)amino]pyridin-2-yl}pyrrolidine-3-carboxylic acid;
(1S,3R)-3-{3-Fluoro-5-[(1-{[4-(trifluoromethyl)phenyl]carbamoyl}-D-prolyl)amino]pyridin-2-yl}cyclopentane-1-carboxylic acid;
(1R,3R)-3-{3-Fluoro-5-[(1-{[4-(trifluoromethyl)phenyl]carbamoyl}-D-prolyl)amino]pyridin-2-yl}cyclopentane-1-carboxylic acid;
(1S,3S)-3-{3-Fluoro-5-[(1-{[4-(trifluoromethyl)phenyl]carbamoyl}-D-prolyl)amino]pyridin-2-yl}cyclopentane-1-carboxylic acid;
(1R,3S)-3-{3-Fluoro-5-[(1-{[4-(trifluoromethyl)phenyl]carbamoyl}-D-prolyl)amino]pyridin-2-yl}cyclopentane-1-carboxylic acid;
(1S,3R)-3-{3-Fluoro-5-[(1-{[4-(trifluoromethoxy)phenyl]carbamoyl}-D-prolyl)amino]pyridin-2-yl}cyclopentane-1-carboxylic acid;
(1R,3R)-3-{3-Fluoro-5-[(1-{[4-(trifluoromethoxy)phenyl]carbamoyl}-D-prolyl)amino]pyridin-2-yl}cyclopentane-1-carboxylic acid;
(1S,3S)-3-{3-Fluoro-5-[(1-{[4-(trifluoromethoxy)phenyl]carbamoyl}-D-prolyl)amino]pyridin-2-yl}cyclopentane-1-carboxylic acid; and
(1R,3S)-3-{3-Fluoro-5-[(1-{[4-(trifluoromethoxy)phenyl]carbamoyl}-D-prolyl)amino]pyridin-2-yl}cyclopentane-1-carboxylic acid,
or a pharmaceutically acceptable salt thereof.

20. A pharmaceutical composition comprising a compound of claim 1 or a pharmaceutically acceptable salt thereof and a pharmaceutically acceptable excipient.

21. A method for weight management or for treating or preventing a condition, disease, or disorder, comprising administering a compound of claim 1 or a pharmaceutically acceptable salt thereof to a human in need thereof, wherein the condition, disease, or disorder is selected from the group consisting of diabetes [e.g. Type 1 diabetes mellitus (T1D), Type 2 diabetes mellitus (T2DM), including pre-diabetes], idiopathic T1D (Type 1b), latent autoimmune diabetes in adults (LADA), early-onset T2DM (EOD), youth-onset atypical diabetes (YOAD), maturity onset diabetes of the young (MODY), malnutrition-related diabetes, gestational diabetes, hyperglycemia, insulin resistance, hepatic insulin resistance, impaired glucose tolerance, diabetic neuropathy, diabetic nephropathy, kidney disease [e.g., acute kidney disorder, tubular dysfunction, proinflammatory changes to the proximal tubules, or chronic kidney disease (CKD)], diabetic retinopathy, adipocyte dysfunction, visceral adipose deposition, sleep apnea [e.g. obstructive sleep apnea (OSA)], obesity (including hypothalamic obesity and monogenic obesity) and related comorbidities (e.g., osteoarthritis and urine incontinence), eating disorders (including binge eating syndrome, bulimia nervosa, and syndromic obesity such as Prader-Willi and Bardet-Biedl syndromes), weight gain such as weight gain caused by use of other agents (e.g., caused by use of steroids and/or antipsychotics, or caused by treatment of depression, or caused by use of agents on cognitive function), overweight, excessive sugar craving, dyslipidemia [including hyperlipidemia, hypertriglyceridemia, increased total cholesterol, high LDL (low-density lipoprotein) cholesterol, and low HDL (high-density lipoprotein) cholesterol], hyperinsulinemia, nonalcoholic fatty liver disease [NAFLD, including related diseases such as steatosis, nonalcoholic steatohepatitis (NASH), fibrosis, cirrhosis, and hepatocellular carcinoma], cardiovascular disease, atherosclerosis (including coronary artery disease), peripheral vascular disease, hypertension, endothelial dysfunction, impaired vascular compliance, heart failure [e.g. congestive heart failure, heart failure with preserved ejection fraction (HFpEF), heart failure with reduced ejection fraction (HFrEF)], myocardial infarction (e.g. necrosis and apoptosis), stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, osteoarthritis, Parkinson's disease, left ventricular hypertrophy, peripheral arterial disease (PAD), macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, polycystic ovary syndrome (PCOS), and addiction (e.g., addition to alcohol, nicotine, and/or drug).

22. A method for modulating a glucose-dependent insulinotropic polypeptide receptor (GIPR) comprising contacting the GIPR with a compound of claim 1 or a pharmaceutically acceptable salt thereof.
